SEQUENCE LISTING (135 sequences)

SEQ ID NO: 1 - protein, 7 aa, Artificial Sequence
  heavy chain CDR1
SGGYYWS

SEQ ID NO: 2 - protein, 7 aa, Artificial Sequence
  heavy chain CDR1
TSGVGVG

SEQ ID NO: 3 - protein, 5 aa, Artificial Sequence
  heavy chain CDR1
SYAMH

SEQ ID NO: 4 - protein, 5 aa, Artificial Sequence
  heavy chain CDR1
SYDMH

SEQ ID NO: 5 - protein, 5 aa, Artificial Sequence
  heavy chain CDR1
SYWMH

SEQ ID NO: 6 - protein, 16 aa, Artificial Sequence
  heavy chain CDR2
YIYYSGSTYYNPSLKS

SEQ ID NO: 7 - protein, 16 aa, Artificial Sequence
  heavy chain CDR2
LIYWNDDKRYSPSLKS

SEQ ID NO: 8 - protein, 17 aa, Artificial Sequence
  heavy chain CDR2
WINAGNGNTKYSQKFQG

SEQ ID NO: 9 - protein, 16 aa, Artificial Sequence
  heavy chain CDR2
AIGTAGDTYYPGSVKG

SEQ ID NO: 10 - protein, 17 aa, Artificial Sequence
  heavy chain CDR2
RINSDGSSTSYADSVKG

SEQ ID NO: 11 - protein, 9 aa, Artificial Sequence
  HYYYYSMDV
HYYYYSMDV

SEQ ID NO: 12 - protein, 9 aa, Artificial Sequence
  heavy chain CDR3
HYYYYYLDV

SEQ ID NO: 13 - protein, 8 aa, Artificial Sequence
  heavy chain CDR3
HYYYYMDV

SEQ ID NO: 14 - protein, 9 aa, Artificial Sequence
  heavy chain CDR3
HYYYYYMDV

SEQ ID NO: 15 - protein, 9 aa, Artificial Sequence
  heavy chain CDR3
KTTSFYFDY

SEQ ID NO: 16 - protein, 10 aa, Artificial Sequence
  heavy chain CDR3
RHMRLSCFDY

SEQ ID NO: 17 - protein, 12 aa, Artificial Sequence
  heavy chain CDR3
EGNGANPDAFDI

SEQ ID NO: 18 - protein, 11 aa, Artificial Sequence
  heavy chain CDR3
DLPGSYWYFDL

SEQ ID NO: 19 - protein, 10 aa, Artificial Sequence
  heavy chain CDR3
GVLLYNWFDP

SEQ ID NO: 20 - protein, 11 aa, Artificial Sequence
  light chain CDR1
RASQSISSYLN

SEQ ID NO: 21 - protein, 7 aa, Artificial Sequence
  light chain CDR2
AASSLQS

SEQ ID NO: 22 - protein, 9 aa, Artificial Sequence
  light chain CDR3
QQSYSTPLT

SEQ ID NO: 23 - protein, 243 aa, Artificial Sequence
  anti-HLA-A*11 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYSMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 24 - protein, 244 aa, Artificial Sequence
  anti-HLA-A*11 scFv
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHRHMRLSCFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 25 - protein, 245 aa, Artificial Sequence
  anti-HLA-A*11 scFv
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAREGNGANPDAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 26 - protein, 243 aa, Artificial Sequence
  anti-HLA-A*11 scFv
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCARDLPGSYWYFDLWGRGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 27 - protein, 243 aa, Artificial Sequence
  anti-HLA-A*11 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYYLDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 28 - protein, 243 aa, Artificial Sequence
  anti-HLA-A*11 scFv
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCCLGVLLYNWFDPWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 29 - protein, 242 aa, Artificial Sequence
  anti-HLA-A*11 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 30 - protein, 243 aa, Artificial Sequence
  anti-HLA-A*11 scFv
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHKTTSFYFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 31 - protein, 243 aa, Artificial Sequence
  anti-HLA-A*11 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 32 - DNA, 729 bp, Artificial Sequence
  anti-HLA-A*11 scFv
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactccatggacgtctggggcaaagggaccacggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 33 - DNA, 732 bp, Artificial Sequence
  anti-HLA-A*11 scFv
cagatcaccctgaaagagtccggccccaccctggtgaaacccacccagaccctgaccctgacatgcaccttcagcggcttcagcctgagcacctctggcgtgggcgtgggctggatcagacagcctcccggcaaggccctggaatggctggccctgatctactggaacgacgacaagcggtacagccccagcctgaagtcccggctgaccatcaccaaggacacctcgaagaaccaggtggtgctgaccatgacaaacatggaccccgtggacaccgccacatattactgtgcacacagacacatgcgtttaagctgttttgactactggggccagggaaccctggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 34 - DNA, 735 bp, Artificial Sequence
  anti-HLA-A*11 scFv
caggtgcagctggtgcagtctggcgccgaagtgaagaaacctggcgcctccgtgaaggtgtcctgcaaggccagcggctacaccttcaccagctacgccatgcactgggttcgacaggcccctggccagagactggaatggatgggctggatcaacgccggcaacggcaacaccaagtacagccagaaattccagggcagagtgaccatcacccgggacaccagcgccagcaccgcctacatggaactgagcagcctgcggagcgaggacaccgctgtgtattactgtgcgagagaaggaaatggtgccaaccctgatgcttttgatatctggggccaagggacaatggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 35 - DNA, 729 bp, Artificial Sequence
  anti-HLA-A*11 scFv
gaagtgcagctggtggaaagcggcggaggcctggtgcagcctggcggcagcctgagactgtcttgcgccgccagcggcttcaccttcagcagctacgacatgcactgggtccgccaggccaccggcaagggactggaatgggtgtccgccatcggcacagccggcgacacttactaccccggcagcgtgaagggccggttcaccatcagcagagagaacgccaagaacagcctgtacctgcagatgaacagccttcgagccggcgataccgccgtgtattactgtgcaagagatctccctggtagctactggtacttcgatctctggggccgtggcaccctggtcactgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 36 - DNA, 729 bp, Artificial Sequence
  anti-HLA-A*11 scFv
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactacctggacgtctggggcaaagggaccacggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 37 - DNA, 729 bp, Artificial Sequence
  anti-HLA-A*11 scFv
gaagtgcagctggtggaaagcggcggaggcctggtgcagcctggcggcagcctgagactgtcttgcgccgccagcggcttcaccttcagcagctactggatgcactgggtccgccaggcccctggcaagggactggtctgggtgtctcgaatcaacagcgacggcagcagcaccagctacgccgacagcgtgaagggccggttcaccatcagccgggacaacgccaagaacaccctgtacctgcagatgaacagcctgcgggccgaggacaccgccgtgtattactgttgtttgggtgttttattatacaactggttcgacccctggggccagggaaccctggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 38 - DNA, 726 bp, Artificial Sequence
  anti-HLA-A*11 scFv
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactacatggacgtctggggcaaagggaccacggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 39 - DNA, 729 bp, Artificial Sequence
  anti-HLA-A*11 scFv
cagatcaccctgaaagagtccggccccaccctggtgaaacccacccagaccctgaccctgacatgcaccttcagcggcttcagcctgagcacctctggcgtgggcgtgggctggatcagacagcctcccggcaaggccctggaatggctggccctgatctactggaacgacgacaagcggtacagccccagcctgaagtcccggctgaccatcaccaaggacacctcgaagaaccaggtggtgctgaccatgacaaacatggaccccgtggacaccgccacatattactgtgcacacaaaacgacgtcgttttactttgactactggggccagggaaccctggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 40 - DNA, 729 bp, Artificial Sequence
  anti-HLA-A*11 scFv
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactacatggacgtctggggcaaagggaccacggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 41 - protein, 365 aa, Homo sapiens
MAVMAPRTLLLLLSGALALTQTWAGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 42 - protein, 119 aa, Artificial Sequence
  anti-HLA-A*11 heavy chain
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYSMDVWGKGTTVTVSS

SEQ ID NO: 43 - protein, 120 aa, Artificial Sequence
  anti-HLA-A*11 heavy chain
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHRHMRLSCFDYWGQGTLVTVSS

SEQ ID NO: 44 - protein, 121 aa, Artificial Sequence
  anti-HLA-A*11 heavy chain
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAREGNGANPDAFDIWGQGTMVTVSS

SEQ ID NO: 45 - protein, 119 aa, Artificial Sequence
  anti-HLA-A*11 heavy chain
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCARDLPGSYWYFDLWGRGTLVTVSS

SEQ ID NO: 46 - protein, 119 aa, Artificial Sequence
  anti-HLA-A*11 heavy chain
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYYLDVWGKGTTVTVSS

SEQ ID NO: 47 - protein, 119 aa, Artificial Sequence
  anti-HLA-A*11 heavy chain
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCCLGVLLYNWFDPWGQGTLVTVSS

SEQ ID NO: 48 - protein, 118 aa, Artificial Sequence
  anti-HLA-A*11 heavy chain
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYMDVWGKGTTVTVSS

SEQ ID NO: 49 - protein, 119 aa, Artificial Sequence
  anti-HLA-A*11 heavy chain
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHKTTSFYFDYWGQGTLVTVSS

SEQ ID NO: 50 - protein, 119 aa, Artificial Sequence
  anti-HLA-A*11 heavy chain
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYYMDVWGKGTTVTVSS

SEQ ID NO: 51 - protein, 107 aa, Artificial Sequence [sequence omitted]

SEQ ID NO: 52 - DNA, 357 bp, Artificial Sequence
  anti-HLA-A*11 heavy chain
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactccatggacgtctggggcaaagggaccacggtcaccgtgtcctca

SEQ ID NO: 53 - DNA, 360 bp, Artificial Sequence
  anti-HLA-A*11 heavy chain
cagatcaccctgaaagagtccggccccaccctggtgaaacccacccagaccctgaccctgacatgcaccttcagcggcttcagcctgagcacctctggcgtgggcgtgggctggatcagacagcctcccggcaaggccctggaatggctggccctgatctactggaacgacgacaagcggtacagccccagcctgaagtcccggctgaccatcaccaaggacacctcgaagaaccaggtggtgctgaccatgacaaacatggaccccgtggacaccgccacatattactgtgcacacagacacatgcgtttaagctgttttgactactggggccagggaaccctggtcaccgtgtcctca

SEQ ID NO: 54 - DNA, 363 bp, Artificial Sequence
  anti-HLA-A*11 heavy chain
caggtgcagctggtgcagtctggcgccgaagtgaagaaacctggcgcctccgtgaaggtgtcctgcaaggccagcggctacaccttcaccagctacgccatgcactgggttcgacaggcccctggccagagactggaatggatgggctggatcaacgccggcaacggcaacaccaagtacagccagaaattccagggcagagtgaccatcacccgggacaccagcgccagcaccgcctacatggaactgagcagcctgcggagcgaggacaccgctgtgtattactgtgcgagagaaggaaatggtgccaaccctgatgcttttgatatctggggccaagggacaatggtcaccgtgtcctca

SEQ ID NO: 55 - DNA, 357 bp, Artificial Sequence
  anti-HLA-A*11 heavy chain
gaagtgcagctggtggaaagcggcggaggcctggtgcagcctggcggcagcctgagactgtcttgcgccgccagcggcttcaccttcagcagctacgacatgcactgggtccgccaggccaccggcaagggactggaatgggtgtccgccatcggcacagccggcgacacttactaccccggcagcgtgaagggccggttcaccatcagcagagagaacgccaagaacagcctgtacctgcagatgaacagccttcgagccggcgataccgccgtgtattactgtgcaagagatctccctggtagctactggtacttcgatctctggggccgtggcaccctggtcactgtgtcctca

SEQ ID NO: 56 - DNA, 357 bp, Artificial Sequence
  anti-HLA-A*11 heavy chain
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactacctggacgtctggggcaaagggaccacggtcaccgtgtcctca

SEQ ID NO: 57 - DNA, 357 bp, Artificial Sequence
  anti-HLA-A*11 heavy chain
gaagtgcagctggtggaaagcggcggaggcctggtgcagcctggcggcagcctgagactgtcttgcgccgccagcggcttcaccttcagcagctactggatgcactgggtccgccaggcccctggcaagggactggtctgggtgtctcgaatcaacagcgacggcagcagcaccagctacgccgacagcgtgaagggccggttcaccatcagccgggacaacgccaagaacaccctgtacctgcagatgaacagcctgcgggccgaggacaccgccgtgtattactgttgtttgggtgttttattatacaactggttcgacccctggggccagggaaccctggtcaccgtgtcctca

SEQ ID NO: 58 - DNA, 354 bp, Artificial Sequence
  anti-HLA-A*11 heavy chain
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactacatggacgtctggggcaaagggaccacggtcaccgtgtcctca

SEQ ID NO: 59 - DNA, 357 bp, Artificial Sequence
  anti-HLA-A*11 heavy chain
cagatcaccctgaaagagtccggccccaccctggtgaaacccacccagaccctgaccctgacatgcaccttcagcggcttcagcctgagcacctctggcgtgggcgtgggctggatcagacagcctcccggcaaggccctggaatggctggccctgatctactggaacgacgacaagcggtacagccccagcctgaagtcccggctgaccatcaccaaggacacctcgaagaaccaggtggtgctgaccatgacaaacatggaccccgtggacaccgccacatattactgtgcacacaaaacgacgtcgttttactttgactactggggccagggaaccctggtcaccgtgtcctca

SEQ ID NO: 60 - DNA, 357 bp, Artificial Sequence
  anti-HLA-A*11 heavy chain
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactacatggacgtctggggcaaagggaccacggtcaccgtgtcctca

SEQ ID NO: 61 - DNA, 321 bp, Artificial Sequence
  anti-HLA-A*11 light chain
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 62 - protein, 650 aa, Homo sapiens
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 63 - protein, 22 aa, Artificial Sequence
  signal peptide
MDMRVPAQLLGLLLLWLRGARC

SEQ ID NO: 64 - protein, 267 aa, Artificial sequence
  chimeric antigen receptor hinge, transmembrane and intracellular domains
TTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 253 aa, Homo sapiens
YGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 66 - protein, 20 aa, Homo sapiens
YGSQSSKPYLLTHPSDPLEL

SEQ ID NO: 67 - protein, 233 aa, Homo sapiens
VVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 68 - protein, 63 aa, Homo sapiens
YGSQSSKPYLLTHPSDPLELVVSGPSGGPSSPTTGPTSTSGPEDQPLTPTGSDPQSGLGRHLG

SEQ ID NO: 69 - protein, 23 aa, Homo sapiens
VVIGILVAVILLLLLLLLLFLIL

SEQ ID NO: 70 - protein, 167 aa, Homo sapiens
RHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 71 - protein, 6 aa, Artificial Sequence
  ITIM sequence
NLYAAV

SEQ ID NO: 72 - protein, 6 aa, Artificial Sequence
  ITIM sequence
VTYAEV

SEQ ID NO: 73 - protein, 6 aa, Artificial Sequence
  ITIM Sequence
VTYAQL

SEQ ID NO: 74 - protein, 6 aa, Artificial Sequence
  ITIM sequence
SIYATL

SEQ ID NO: 75 - protein, 35 aa, Homo sapiens
NLYAAVKHTQPEDGVEMDTRSPHDEDPQAVTYAEV

SEQ ID NO: 76 - protein, 58 aa, Homo sapiens
VTYAEVKHSRPRREMASPPSPLSGEFLDTKDRQAEEDRQMDTEAAASEAPQDVTYAQL

SEQ ID NO: 77 - protein, 36 aa, Homo sapiens
VTYAQLHSLTLRREATEPPPSQEGPSPAVPSIYATL

SEQ ID NO: 78 - protein, 87 aa, Homo sapiens
NLYAAVKHTQPEDGVEMDTRSPHDEDPQAVTYAEVKHSRPRREMASPPSPLSGEFLDTKDRQAEEDRQMDTEAAASEAPQDVTYAQL

SEQ ID NO: 79 - protein, 88 aa, Homo sapiens
VTYAEVKHSRPRREMASPPSPLSGEFLDTKDRQAEEDRQMDTEAAASEAPQDVTYAQLHSLTLRREATEPPPSQEGPSPAVPSIYATL

SEQ ID NO: 80 - protein, 117 aa, Homo sapiens
NLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATL

SEQ ID NO: 81 - protein, 20 aa, Homo sapiens
YGSQSSKPYLLTHPSDPLEL

SEQ ID NO: 82 - protein, 43 aa, Artificial Sequence
  LIR1 based hinge
VVSGPSGGPSSPTTGPTSTSGPEDQPLTPTGSDPQSGLGRHLG

SEQ ID NO: 83 - protein, 64 aa, Artificial Sequence
  LIR1 based hinge
YGSQSSKPYLLTHPSDPLELVVSGPSGGPSSPTTGPTSTSGPEDQPLTPTGSDPQSGLGRHLGV

SEQ ID NO: 84 - protein, 44 aa, Artificial Sequence
  LIR1 based hinge
VVSGPSGGPSSPTTGPTSTSGPEDQPLTPTGSDPQSGLGRHLGV

SEQ ID NO: 85 - protein, 67 aa, Artificial Sequence
  LIR1 based hinge
AGSGGSGGSGGSPVPSTPPTPSPSTPPTPSPSGGSGNSSGSGGSPVPSTPPTPSPSTPPTPSPSASV

SEQ ID NO: 86 - protein, 75 aa, Artificial Sequence
  LIR1 based hinge
AGSGGSGGSGGSPVPSTPPTNSSSTPPTPSPSPVPSTPPTNSSSTPPTPSPSPVPSTPPTNSSSTPPTPSPSASV

SEQ ID NO: 87 - protein, 85 aa, Artificial Sequence
  LIR1 based hinge
VVSGPSGGPSSPTTGPTSTSGPEDQPLTPTGSDPQSGLGRHVVSGPSGGPSSPTTGPTSTSGPEDQPLTPTGSDPQSGLGRHLGV

SEQ ID NO: 88 - protein, 86 aa, Homo sapiens
YGSQSSKPYLLTHPSDPLELVVSGPSGGPSSPTTGPTSTSGPEDQPLTPTGSDPQSGLGRHLGVVIGILVAVILLLLLLLLLFLIL

SEQ ID NO: 89 - protein, 190 aa, Homo sapiens
VVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 90 - protein, 605 aa, Artificial Sequence
  Positive Control TCR (TRAV3-3*01/TRBV4*01)
MKTVTGPLFLCFWLQLNCVSRGEQVEQRPPHLSVREGDSAVITCTYTDPNSYYFFWYKQEPGASLQLLMKVFSSTEINEGQGFTVLLNKKDKRLSLNLTAAHPGDSAAYFCAVSGGTNSAGNKLTFGIGTRVLVRPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSRAKRSGSGATNFSLLKQAGDVEENPGPMGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSRDWGPAEQFFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 91 - protein, 532 aa, Artificial Sequence
  HLA-A*11 CAR
MDMRVPAQLLGLLLLWLRGARCQVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 532 aa, Artificial Sequence
  HLA-A*11 CAR
MDMRVPAQLLGLLLLWLRGARCQITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHKTTSFYFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 531 aa, Artificial Sequence
  HLA-A*11 CAR
MDMRVPAQLLGLLLLWLRGARCQVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - protein, 532 aa, Artificial Sequence
  HLA-A*11 CAR
MDMRVPAQLLGLLLLWLRGARCEVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCCLGVLLYNWFDPWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - protein, 532 aa, Artificial Sequence
  HLA-A*11 CAR
MDMRVPAQLLGLLLLWLRGARCQVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYYLDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - protein, 532 aa, Artificial Sequence
  HLA-A*11 CAR
MDMRVPAQLLGLLLLWLRGARCEVQLVESGGGLVQPGGSLRLSCAASGFTFSSYDMHWVRQATGKGLEWVSAIGTAGDTYYPGSVKGRFTISRENAKNSLYLQMNSLRAGDTAVYYCARDLPGSYWYFDLWGRGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 97 - protein, 534 aa, Artificial Sequence
  HLA-A*11 CAR
MDMRVPAQLLGLLLLWLRGARCQVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAREGNGANPDAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 533 aa, Artificial Sequence
  HLA-A*11 CAR
MDMRVPAQLLGLLLLWLRGARCQITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHRHMRLSCFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - protein, 532 aa, Artificial Sequence
  HLA-A*11 CAR
MDMRVPAQLLGLLLLWLRGARCQVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYSMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - DNA, 1818 bp, Artificial Sequence
  Positive Control TCR (TRAV3-3*01/TRBV4*01)
atgaagacggtgactggacctttgttcctgtgcttctggctgcagctgaactgtgtgagcagaggcgagcaggtggagcagcgccctcctcacctgagtgtccgggagggagacagtgccgttatcacctgcacctacacagaccctaacagttattacttcttctggtacaagcaagagccgggggcaagtcttcagttgcttatgaaggttttctcaagtacggaaataaacgaaggacaaggattcactgtcctactgaacaagaaagacaaacgactctctctgaacctcacagctgcccatcctggggactcagccgcgtacttctgcgcagtcagtggagggactaacagtgcagggaacaagctaacttttggaattggaaccagggtgctggtcaggccagacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagtcgggccaagcggtccggatccggagccaccaacttcagcctgctgaagcaggccggcgacgtggaggagaaccccggccccatgggctgtaggctcctaagctgtgtggccttctgcctcttgggaataggccctttggagacagctgttttccagactccaaactatcatgtcacacaggtgggaaatgaagtgtctttcaattgtaagcaaactctgggccacgatactatgtattggtacaagcaagactctaagaaattgctgaagattatgtttagctacaataataagcaactcattgtaaacgaaacagttccaaggcgcttctcacctcagtcttcagataaagctcatttgaatcttcgaatcaagtctgtagagccggaggactctgctgtgtatctctgtgccagcagtcgggactgggggcctgctgagcagttcttcggaccagggacacgactcaccgtcctagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattcctag

SEQ ID NO: 101 - DNA, 1596 bp, Artificial Sequence
  HLA-A*11 CAR
atggacatgagggtccccgctcagctcctggggctcctgctactctggctccgaggtgccagatgtcaggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactacatggacgtctggggcaaagggaccacggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 102 - DNA, 1596 bp, Artificial Sequence
  HLA-A*11 CAR
atggacatgagggtccccgctcagctcctggggctcctgctactctggctccgaggtgccagatgtcagatcaccctgaaagagtccggccccaccctggtgaaacccacccagaccctgaccctgacatgcaccttcagcggcttcagcctgagcacctctggcgtgggcgtgggctggatcagacagcctcccggcaaggccctggaatggctggccctgatctactggaacgacgacaagcggtacagccccagcctgaagtcccggctgaccatcaccaaggacacctcgaagaaccaggtggtgctgaccatgacaaacatggaccccgtggacaccgccacatattactgtgcacacaaaacgacgtcgttttactttgactactggggccagggaaccctggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 103 - DNA, 1593 bp, Artificial Sequence
  HLA-A*11 CAR
atggacatgagggtccccgctcagctcctggggctcctgctactctggctccgaggtgccagatgtcaggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactacatggacgtctggggcaaagggaccacggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 104 - DNA, 1596 bp, Artificial Sequence
  HLA-A*11 CAR
atggacatgagggtccccgctcagctcctggggctcctgctactctggctccgaggtgccagatgtgaagtgcagctggtggaaagcggcggaggcctggtgcagcctggcggcagcctgagactgtcttgcgccgccagcggcttcaccttcagcagctactggatgcactgggtccgccaggcccctggcaagggactggtctgggtgtctcgaatcaacagcgacggcagcagcaccagctacgccgacagcgtgaagggccggttcaccatcagccgggacaacgccaagaacaccctgtacctgcagatgaacagcctgcgggccgaggacaccgccgtgtattactgttgtttgggtgttttattatacaactggttcgacccctggggccagggaaccctggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 105 - DNA, 1596 bp, Artificial Sequence
  HLA-A*11 CAR
atggacatgagggtccccgctcagctcctggggctcctgctactctggctccgaggtgccagatgtcaggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactacctggacgtctggggcaaagggaccacggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 106 - DNA, 1596 bp, Artificial Sequence
  HLA-A*11 CAR
atggacatgagggtccccgctcagctcctggggctcctgctactctggctccgaggtgccagatgtgaagtgcagctggtggaaagcggcggaggcctggtgcagcctggcggcagcctgagactgtcttgcgccgccagcggcttcaccttcagcagctacgacatgcactgggtccgccaggccaccggcaagggactggaatgggtgtccgccatcggcacagccggcgacacttactaccccggcagcgtgaagggccggttcaccatcagcagagagaacgccaagaacagcctgtacctgcagatgaacagccttcgagccggcgataccgccgtgtattactgtgcaagagatctccctggtagctactggtacttcgatctctggggccgtggcaccctggtcactgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 107 - DNA, 1602 bp, Artificial Sequence
  HLA-A*11 CAR
atggacatgagggtccccgctcagctcctggggctcctgctactctggctccgaggtgccagatgtcaggtgcagctggtgcagtctggcgccgaagtgaagaaacctggcgcctccgtgaaggtgtcctgcaaggccagcggctacaccttcaccagctacgccatgcactgggttcgacaggcccctggccagagactggaatggatgggctggatcaacgccggcaacggcaacaccaagtacagccagaaattccagggcagagtgaccatcacccgggacaccagcgccagcaccgcctacatggaactgagcagcctgcggagcgaggacaccgctgtgtattactgtgcgagagaaggaaatggtgccaaccctgatgcttttgatatctggggccaagggacaatggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 108 - DNA, 1599 bp, Artificial Sequence
  HLA-A*11 CAR
atggacatgagggtccccgctcagctcctggggctcctgctactctggctccgaggtgccagatgtcagatcaccctgaaagagtccggccccaccctggtgaaacccacccagaccctgaccctgacatgcaccttcagcggcttcagcctgagcacctctggcgtgggcgtgggctggatcagacagcctcccggcaaggccctggaatggctggccctgatctactggaacgacgacaagcggtacagccccagcctgaagtcccggctgaccatcaccaaggacacctcgaagaaccaggtggtgctgaccatgacaaacatggaccccgtggacaccgccacatattactgtgcacacagacacatgcgtttaagctgttttgactactggggccagggaaccctggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 109 - DNA, 1596 bp, Artificial Sequence
  HLA-A*11 CAR
atggacatgagggtccccgctcagctcctggggctcctgctactctggctccgaggtgccagatgtcaggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactccatggacgtctggggcaaagggaccacggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 110 - protein, 10 aa, Artificial Sequence
  peptide antigen
VVVGAVGVGK

SEQ ID NO: 111 - protein, 518 aa, Artificial Sequence
  HLA-A*11 LIR1 inhibitory receptor
MDMRVPAQLLGLLLLWLRGARCEVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCCLGVLLYNWFDPWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 112 - DNA, 1554 bp, Artificial Sequence
  HLA-A*11 LIR1 inhibitory receptor
atggacatgagggtccccgctcagctcctggggctcctgctactctggctccgaggtgccagatgtgaagtgcagctggtggaaagcggcggaggcctggtgcagcctggcggcagcctgagactgtcttgcgccgccagcggcttcaccttcagcagctactggatgcactgggtccgccaggcccctggcaagggactggtctgggtgtctcgaatcaacagcgacggcagcagcaccagctacgccgacagcgtgaagggccggttcaccatcagccgggacaacgccaagaacaccctgtacctgcagatgaacagcctgcgggccgaggacaccgccgtgtattactgttgtttgggtgttttattatacaactggttcgacccctggggccagggaaccctggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaagtacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccac

SEQ ID NO: 113 - protein, 23 aa, Homo sapiens
VIGFRILLLKVAGFNLLMTLRLW

SEQ ID NO: 114 - protein, 23 aa, Homo sapiens
TILYEILLGKATLYAVLVSALVL

SEQ ID NO: 115 - protein, 27 aa, Homo sapiens
FWVLVVVGGVLACYSLLVTVAFIIFWV

SEQ ID NO: 116 - protein, 45 aa, Homo sapiens
TTTPAPRPPTPAPTIASQPLSLRPEACRPAAGGAVHTRGLDFACD

SEQ ID NO: 117 - protein, 41 aa, Homo sapiens
CTIEVMYPPPYLDNEKSNGTIIHVKGKHLCPSPLFPGPSKP

SEQ ID NO: 118 - protein, 455 aa, Homo sapiens
MSLTVVSMACVGFFLLQGAWPLMGGQDKPFLSARPSTVVPRGGHVALQCHYRRGFNNFMLYKEDRSHVPIFHGRIFQESFIMGPVTPAHAGTYRCRGSRPHSLTGWSAPSNPLVIMVTGNHRKPSLLAHPGPLLKSGETVILQCWSDVMFEHFFLHREGISEDPSRLVGQIHDGVSKANFSIGPLMPVLAGTYRCYGSVPHSPYQLSAPSDPLDIVITGLYEKPSLSAQPGPTVQAGENVTLSCSSWSSYDIYHLSREGEAHERRLRAVPKVNRTFQADFPLGPATHGGTYRCFGSFHALPCVWSNSSDPLLVSVTGNPSSSWPSPTEPSSKSGICRHLHVLIGTSVVIFLFILLLFFLLYRWCSNKKNAAVMDQEPAGDRTVNRQDSDEQDPQEVTYAQLDHCVFIQRKISRPSQRPKTPLTDTSVYTELPNAEPRSKVVSCPRAPQSGLEGVF

SEQ ID NO: 119 - protein, 90 aa, Homo sapiens
NKKNAAVMDQEPAGDRTVNRQDSDEQDPQEVTYAQLDHCVFIQRKISRPSQRPKTPLTDTSVYTELPNAEPRSKVVSCPRAPQSGLEGVF

SEQ ID NO: 120 - protein, 410 aa, Homo sapiens [sequence omitted]

SEQ ID NO: 121 - protein, 93 aa, Homo sapiens
FFYGSISRAEAEEHLKLAGMADGLFLLRQCLRSLGGYVLSLVHDVRFHHFPIERQLNGTYAIAGGKAHCGPAELCEFYSRDPDGLPCNLRKPC

SEQ ID NO: 122 - protein, 92 aa, Homo sapiens
WYHSSLTREEAERKLYSGAQTDGKFLLRPRKEQGTYALSLIYGKTVYHYLISQDKAGKYCIPEGTKFDTLWQLVEYLKLKADGLIYCLKEAC

SEQ ID NO: 123 - protein, 619 aa, Homo sapiens
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 124 - protein, 116 aa, Homo sapiens
LRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 125 - protein, 167 aa, Homo sapiens
RRQRHSKHRTSDQRKTDFQRPAGAAETEPKDRGLLRRSSPAADVQEENLYAAVKDTQSEDRVELDSQSPHDEDPQAVTYAPVKHSSPRREMASPPSSLSGEFLDTKDRQVEEDRQMDTEAAASEASQDVTYAQLHSLTLRRKATEPPPSQEGEPPAEPSIYATLAIH

SEQ ID NO: 126 - protein, 168 aa, Homo sapiens
QHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYATLAIH

SEQ ID NO: 127 - protein, 111 aa, Homo sapiens
RHRHQSKHRTSAHFYRPAGAAGPEPKDQGLQKRASPVADIQEEILNAAVKDTQPKDGVEMDARAAASEAPQDVTYAQLHSLTLRREATEPPPSQEREPPAEPSIYAPLAIH

SEQ ID NO: 128 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 129 - protein, 144 aa, Homo sapiens
TERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 130 - protein, 95 aa, Homo sapiens
RAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 131 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 132 - protein, 216 aa, Homo sapiens
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 133 - protein, 310 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 134 - protein, 85 aa, Artificial Sequence
  inhibitory intracellular domain
RVKFSRSADAPAYQQPDPQEVTYAQLDKRRGRDPEMGGKPRRKPDPQEVTYAQLGMKGERRRGKGHPDPQEVTYAQLHMQALPPR

SEQ ID NO: 135 - protein, 32 aa, Homo sapiens
TTGPTSTSGPEDQPLTPTGSDPQSGLGRHLGV

(19) United States
(12) Patent Application Publication
(10) Pub. No.: US 2024/0180969 A1
(43) Pub. Date: Jun. 6, 2024

(54) POLYPEPTIDES TARGETING HLA-A*11 AND METHODS OF USE THEREOF

(71) Applicant: A2 Biotherapeutics, Inc., Agoura Hills, CA (US)

(72) Inventors: Carl Alexander Kamb, Thousand Oaks, CA (US); Agnes E. Hamburger, Newbury Park, CA (US)

(21) Appl. No.: 18/553,326

(22) PCT Filed: Apr. 1, 2022
(86) PCT No.: PCT/US2022/023071

§ 371 Date: Sep. 29, 2023

Related U.S. Application Data
(60) Provisional application No. 63/169,484, filed on Apr. 1, 2021.

(51) Int. Cl. A61K 35/17; A61K 39/00; A61P 35/00; C07K 16/28; C12N 5/0783; C12N 15/66; G01N 33/574

(52) U.S. Cl. CPC A61K 35/17; A61K 39/4611; A61K 39/4631; A61K 39/464411; A61P 35/00; C07K 16/2833; C12N 5/0636; C12N 15/66; G01N 33/57492; A61K 2239/13; A61K 2239/17; A61K 2239/21; A61K 2239/22; G01N 2474/20

(57) ABSTRACT

The disclosure relates to antigen binding domains that specifically bind to a major histocompatibility class I (MHC I) complex comprising an a chain encoded by HLA-A*11 alleles. The disclosure further relates to antibodies and receptors comprising said antigen binding domains, and their use in diagnostics and adoptive cell therapy.

### CROSS REFERENCE TO RELATED APPLICATIONS

This application claims priority to and the benefit of U.S. Provisional Application No. 63/169,484, filed Apr. 1, 2021, which is incorporated herein by reference in its entirety.

### REFERENCE TO SEQUENCE LISTING SUBMITTED ELECTRONICALLY

The contents of the text filed named “A2BI-037_01WO_Seq_List_ST25.txt”, which was created on Mar. 30, 2022 and is 192,641 bytes in size, are hereby incorporated by reference in their entirety.

### BACKGROUND

Human leukocyte antigen (HLA) is a subunit of the major histocompatibility complex (MHC) class I (MHC-I), which are encoded by a set of linked, polymorphic genes. MHC is involved in the binding and presentation of antigens on the cell surface for recognition by immune cells. MHC-I molecules, including HLA-A, are highly polymorphic. For example, there are at least 6,425 HLA-A alleles known in humans, which encode at least 3,929 HLA-A proteins. Human leukocyte antigen A*11 (HLA-A*11) is a human leukocyte antigen encoded by the HLA-A locus with a serotype within the HLA-A serotype group. The disclosure provides antigen binding domains that can specifically target A*11 alleles of HLA-A.

### SUMMARY

The disclosure provides polypeptides with an antigen binding domain that specifically binds to a major histocompatibility compatibility class I (MHC I) complex comprising a human leukocyte antigen a chain encoded by an HLA-A*11 allele (HLA-A*11).

In some embodiments of the polypeptides of the disclosure, the antigen binding domain is a human antibody or an antigen-binding fragment thereof. In some embodiments, the antigen binding domain comprises a single chain variable fragment (scFv), a single chain Fab (scFab), a single domain antibody (sdAb), a fragment antigen binding (Fab), a F(ab′)₂, or a Fab′. In some embodiments, the antigen binding domain is a scFv. In some embodiments, the scFv comprises a variable heavy chain (VH)-linker-variable light chain (VL) or a VL-linker-VH orientation.

In some embodiments of the polypeptides of the disclosure, the antigen binding domain comprises: (a) a heavy chain (HC) complementarity determining region 1 (CDR1) sequence selected from the group consisting of SGGYYWS (SEQ ID NO: 1), TSGVGVG (SEQ ID NO: 2), SYAMH (SEQ ID NO: 3), SYDMH (SEQ ID NO: 4), and SYWMH (SEQ ID NO: 5); (b) a HC CDR2 sequence selected from the group consisting of YIYYSGSTYYNPSLKS (SEQ ID NO: 6), LIYWNDDKRYSPSLKS (SEQ ID NO: 7), WINAGNGNTKYSQKFQG (SEQ ID NO: 8), AIGTAGDTYYPGSVKG (SEQ ID NO: 9), and RINSDGSSTSYADSVKG (SEQ ID NO: 10); and (c) a HC CDR3 sequence selected from the group consisting of HYYYYSMDV (SEQ ID NO: 11), HYYYYYLDV (SEQ ID NO: 12), HYYYYMDV (SEQ ID NO: 13), HYYYYYMDV (SEQ ID NO: 14), KTTSFYFDY (SEQ ID NO: 15), RHMRLSCFDY (SEQ ID NO: 16), EGNGANPDAFDI (SEQ ID NO: 17), DLPGSYWYFDL (SEQ ID NO: 18), and GVLLYNWFDP (SEQ ID NO: 19). In some embodiments, the antigen binding domain comprises a light chain (LC) complementarity determining region 1 (CDR1) comprising a sequence of RASQSISSYLN (SEQ ID NO: 20), a LC CDR2 comprising a sequence of AASSLQS (SEQ ID NO: 21) and a LC CDR3 comprising a sequence of QQSYSTPLT (SEQ ID NO: 22).

In some embodiments of the polypeptides of the disclosure, the antigen binding domain comprises a HC CDR1 comprising TSGVGVG (SEQ ID NO: 2), a HC CDR2 comprising LIYWNDDKRYSPSLKS (SEQ ID NO: 7), a HC CDR3 comprising KTTSFYFDY (SEQ ID NO: 15), a LC CDR1 comprising RASQSISSYLN (SEQ ID NO: 20), a LC CDR2 comprising AASSLQS (SEQ ID NO: 21), and a LC CDR3 comprising QQSYSTPLT (SEQ ID NO: 22).

In some embodiments of the polypeptides of the disclosure, the antigen binding domain comprises a HC CDR1 comprising SYWMH (SEQ ID NO: 5), a HC CDR2 comprising RINSDGSSTSYADSVKG (SEQ ID NO: 10), a HC CDR comprising GVLLYNWFDP (SEQ ID NO: 19), a LC CDR1 comprising RASQSISSYLN (SEQ ID NO: 20), a LC CDR2 comprising AASSLQS (SEQ ID NO: 21), and a LC CDR3 comprising QQSYSTPLT (SEQ ID NO: 22).

In some embodiments of the polypeptides of the disclosure, the antigen binding domain comprises a variable heavy chain comprising a sequence of SEQ ID NOS: 42-50. In some embodiments, the antigen binding domain comprises a variable light chain comprising a sequence of SEQ ID NO: 51. In some embodiments, the antigen binding domain comprises a variable heavy chain of SEQ ID NO: 47 and a variable light chain of SEQ ID NO: 51. In some embodiments, the antigen binding domain comprises a variable heavy chain of SEQ ID NO: 49 and a variable light chain of SEQ ID NO: 51.

In some embodiments of the polypeptides of the disclosure, the antigen binding domain comprises a sequence of SEQ ID NOS: 23-31. In some embodiments, the antigen binding domain comprises a sequence of SEQ ID NO: 28 or SEQ ID NO: 30.

In some embodiments of the polypeptides of the disclosure, the polypeptide comprises a monoclonal antibody. In some embodiments, the polypeptide comprises a multispecific antibody.

The disclosure provides receptors comprising the polypeptides of the disclosure.

In some embodiments of the receptors of the disclosure, the receptor provides an activating signal to a cell. In some embodiments, the cell is an immune cell. In some embodiments, the receptor is a chimeric antigen receptor (CAR) or a T cell receptor (TCR).

In some embodiments of the receptors of the disclosure, the receptor is a CAR. In some embodiments, the CAR comprises an extracellular antigen binding domain, a transmembrane domain, and one or more intracellular domains, and wherein the extracellular antigen-binding domain comprises the polypeptide. In some embodiments, the transmembrane domain comprises a transmembrane domain isolated or derived from CD8a molecule (CD8α), CD4 molecule (CD4), CD28 molecule (CD28), TNF receptor superfamily member 9 (CD137, or 4-1BB), CD80 molecule (CD80), CD86 molecule (CD86), cytotoxic T-lymphocyte associated protein 4 (CD152), programmed cell death 1 (PD-1), CD247 molecule (CD3ζ), or Fc fragment of IgE receptor Ig (FcRγ). In some embodiments, the one or more intracellular domains comprise an intracellular signaling domain isolated or derived from an immune effector cell protein. In some embodiments, the intracellular signaling domain comprises an intracellular signaling domain isolated or derived from CD3ζ. In some embodiments, the CAR comprises a co-stimulatory domain. In some embodiments, the co-stimulatory domain comprises a co-stimulatory domain isolated or derived from CD27 molecule (CD27), CD28, CD137, TNF receptor superfamily member 4 (OX40), TNF receptor superfamily member 8 (CD30), CD40 molecule (CD40), CD40 ligand (CD40L), CD3ζ, integrin subunit beta 2 (LFA-1), inducible T cell costimulator (ICOS), CD2 molecule (CD2), CD7 molecule (CD7), TNF superfamily member 14 (LIGHT), killer cell lectin like receptor C2 (NKG2C), CD276 molecule (B7-H3), or hematopoietic cell signal transducer (DAP10). In some embodiments, the one or more intracellular domains comprise intracellular domains isolated or derived from CD28, 4-1BB and CD3ζ. In some embodiments, the CAR comprises a hinge domain between the extracellular domain and the transmembrane domain. In some embodiments, the hinge domain is isolated or derived from CD4, CD8α, IgG1, IgG2, or IgG4. In some embodiments, the CAR comprises a signal peptide. In some embodiments, the signal peptide comprises a sequence of MDMRVPAQLLGLLLLWLRGARC (SEQ ID NO: 63).

In some embodiments of the receptors of the disclosure, the receptor is a CAR. In some embodiments, the hinge, transmembrane, and intracellular domains of the CAR comprise a sequence at least 90%, at least 95%, at least 99%, or 100% identical to SEQ ID NO: 64.

In some embodiments of the receptors of the disclosure, the receptor is a TCR. In some embodiments, the TCR comprises an extracellular antigen-binding domain comprising the polypeptide. In some embodiments, the antigen binding domain is fused to one or more of a TCRα, TCRβ, CD3ζ, CD3δ, CD3ε or CD3γ subunits of the TCR.

In some embodiments the receptors of the disclosure, the receptor provides an inhibitory signal to a cell. In some embodiments, the cell is an immune cell. In some embodiments, the receptor is an inhibitory CAR or a TCR. In some embodiments, the receptor comprises an extracellular domain comprising the antibody or antigen-binding fragment thereof and an inhibitory intracellular domain. In some embodiments, the inhibitory intracellular domain is isolated or derived from leukocyte immunoglobulin like receptor B1 (LILRB1). In some embodiments, the receptor comprises a transmembrane domain. In some embodiments, the transmembrane domain is isolated or derived from TCRa, TCRb, or LILRB1. In some embodiments, the receptor further comprises an extracellular hinge domain. In some embodiments, the extracellular hinge domain comprises a hinge domain isolated or derived from LILRB1. In some embodiments, the hinge, transmembrane, and intracellular domains comprise a sequence at least 90%, at least 95%, at least 99%, or 100% identical to SEQ ID NO: 65.

The disclosure provides nucleic acids encoding the polypeptides or receptors of the disclosure.

The disclosure provides vectors comprising the nucleic acids of the disclosure.

The disclosure provides cells comprising the nucleic acids or vectors of the disclosure.

The disclosure provides cells comprising the nucleic acids or vectors of the disclosure.

The disclosure provides recombinant immune cells expressing the receptors of the disclosure. In some embodiments, the immune cells are T cells or NK cells.

The disclosure provides pharmaceutical compositions comprising the polypeptides or receptors of the disclosure, and a pharmaceutically acceptable carrier, diluent, or excipient.

The disclosure provides pharmaceutical compositions comprising a plurality of the recombinant immune cell of the disclosure, and a pharmaceutically acceptable carrier, diluent, or excipient.

The disclosure provides methods for treating a cancer a subject in need thereof, the method comprising administering a therapeutically effective amount of the recombinant cells of the disclosure, or the pharmaceutical composition comprising same to the subject, wherein cells of the cancer have lost expression of HLA-A*11 due to loss of heterozygosity.

In some embodiments of the methods of the disclosure, the methods further comprise (a) determining if the subject is heterozygous for an HLA-A*11 allele; (b) isolating a plurality of cancer cells from the subject; (c) detecting the presence or absence of HLA-A*11 on the cancer cells using the polypeptides of the disclosure; and (e) administering the recombinant cell or pharmaceutical composition when the plurality of cancer cells do not express HLA-A*11. In some embodiments, the cancer comprises a liquid tumor or a solid tumor.

The disclosure provides methods for determining whether cancer cells express HLA-A*11, comprising (a) providing a plurality of cancer cells; and (b) detecting the presence of absence of HLA-A*11 on the cancer cells using the polypeptides of the disclosure. In some embodiments, the detecting at step (b) comprises immunohistochemistry.

The disclosure provides methods for making a recombinant immune cell, comprising: (a) providing a plurality of immune cells; and (b) transforming the plurality of immune cells with the nucleic acid or vector of the disclosure.

The disclosure provides methods for making a polypeptide, comprising: (a) contacting the nucleic acid of claim or vector of the disclosure with a cell; (b) culturing the cell under conditions whereby the polypeptide is expressed by the cell; and (c) purifying the polypeptide.

The disclosure provides kits comprising the polypeptides, nucleic acids, vectors, cells, recombinant immune cells, or pharmaceutical compositions of the disclosure.

### BRIEF DESCRIPTION OF THE DRAWINGS

FIG. 1 is a set of plots showing the enrichment of anti-HLA-A*11 binders through multiple rounds of cell sorting.

FIG. 2 is a plot showing Jurkat NFAT luciferase (JNL) cell activation in a peptide titration assay using T2 cells transduced with HLA-A*11.

FIGS. 3A-3B are each a set of histograms showing staining of Jurkat NFAT luciferase (JNL) cells transfected with indicated CAR constructs, as well as a positive TCR control and a negative vector only control (FIG. 3A). Protein L or an anti-murine TCRb antibody was used to stain for surface expression, and an HLA-A*11 pMHC tetramer was used to stain for ligand binding.

FIG. 4 is a plot showing Jurkat NFAT luciferase (JNL) cell activation in an mRNA titration assay. HeLa target cells were transfected with serially diluted HLA-A*11 mRNA and JNL cells were transiently transfected to express CARs. The functional response (RLU) was assessed after a 6 hour co-culture.

FIG. 5 is a set of histograms showing A*11 mRNA titrated in HeLa cells. QIFIKIT (quantitative analysis kit, Agilent) was used to convert flow-cytometry based surface expression to molecules of HLA-A*11/cell.

FIG. 6A is a plot showing Jurkat NFAT luciferase (JNL) cell activation in an mRNA titration assay using HeLa cells transfected with mRNA encoding HLA-A*11. HeLa cells were transfected with serially-diluted HLA-A*11 mRNA, and JNL cells were transiently transfected with an activating CAR+/−HLA-A*11 inhibitory receptor with scFv HLA-A*11 #4. The functional response was assessed after a 6 hour co-culture.

FIG. 6B shows the molecule/cell sensitivity (IC50) of the inhibitory receptor with HLA-A*11 #4.

### DETAILED DESCRIPTION

The present disclosure provides novel antibodies and antigen-binding fragments thereof that selectively bind to HLA-A*11. Also provided are receptors that bind to this target. The receptors can be chimeric antigen receptors (CARs) or engineered T cell receptors (TCRs). Whether CAR or TCR, the receptors, depending on their intracellular domains and other elements, can act to either activate an immune cell, or inhibit an immune cell, upon binding of HLA-A*11 antigen. These antibodies and receptors may be used to treat cancer. In the context of the present disclosure, the following definitions are provided.

### Definitions

As used in the specification and claims, the singular form “a”, “an” and “the” include plural references unless the context clearly dictates otherwise. For example, the term “a cell” includes a plurality of cells, including mixtures thereof.

As used herein, the “administration” of an agent, e.g., an anti-HLA-A*11 antibody or CAR-expressing cell, to a subject or subject includes any route of introducing or delivering to a subject a compound to perform its intended function. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and target cell or tissue. Non-limiting examples of route of administration include parenteral, enteral, and topical routes of administration. Administration includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

As used herein, the term “animal” refers to living multi-cellular vertebrate organisms, a category that includes, for example, mammals and birds. The term “mammal” includes both human and non-human mammals. Similarly, the term “subject” or “patient” includes both human and veterinary subjects, for example, humans, non-human primates, dogs, cats, sheep, mice, horses, and cows.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. The term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 104 M⁻¹ greater or at least 105 M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology 3^{rd}, Ed., W.H. Freeman & Co., New York, 1997. The term antibody herein is used in the broadest sense and specifically covers intact monoclonal antibodies, polyclonal antibodies, multispecific antibodies (e.g. bispecific antibodies) formed from at least two intact antibodies, and antibody fragments, so long as they exhibit the desired biological activity.

“Antibody fragments” or “antigen binding fragments” include proteolytic antibody fragments (such as F(ab′)2 fragments, Fab′ fragments, Fab′-SH fragments and Fab fragments as are known in the art), recombinant antibody fragments (such as sFv fragments, dsFv fragments, bispecific sFv fragments, bispecific dsFv fragments, F(ab)′2 fragments, single chain Fv proteins (“scFv”), disulfide stabilized Fv proteins (“dsFv”), diabodies, and triabodies (as are known in the art), and camelid antibodies (see, for example, U.S. Pat. Nos. 6,015,695; 6,005,079; 5,874,541; 5,840,526; 5,800,988; and 5,759,808). An scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains.

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

As used herein, “binding affinity” refers to the tendency of one molecule to bind (typically non-covalently) with another molecule, such as the tendency of a member of a specific binding pair for another member of a specific binding pair. A binding affinity can be measured as a dissociation constant, which for a specific binding pair (such as an antibody/antigen pair) can be lower than 1×10⁻⁵ M, lower than 1×10⁻⁶ M, lower than 1×10⁻⁷ M, lower than 1×10⁻⁸ M, lower than 1×10⁻⁹ M, lower than 1×10⁻¹⁰ M, lower than 1×10⁻¹¹ M or lower than 1×10⁻¹² M. In one aspect, binding affinity is calculated by a modification of the Scatchard method described by Frankel et al., Mol. Immunol., 16:101-106, 1979. In another aspect, binding affinity is measured by a binding constant. In another aspect, binding affinity is measured by an antigen/antibody dissociation rate. In yet another aspect, a high binding affinity is measured by a competition radioimmunoassay.

The term “specific” or “specificity” refers to the ability of a molecule to bind to a unique epitope. For example, with antibodies or antigen binding domains derived from antibodies, specificity can either be viewed as a measure of the goodness of fit between the antibody-combining site (paratope) and the corresponding antigenic determinant (epitope), or the ability of the antibody to discriminate between similar or even dissimilar antigens.

The terms “cancer,” “neoplasm,” and “tumor,” used interchangeably and in either the singular or plural form, refer to cells that have undergone a malignant transformation that makes them pathological to the host organism. Non-limiting examples of cancers that may be treated according to the methods of the present disclosure include hematological malignancies and solid tumors.

As used herein, “activator receptor” refers to a receptor that, in response to its cognate ligand, transduces a signal that activates one or more cellular responses of an immune cell expressing the activator receptor.

“Inhibitory receptor” or “blocker receptor” refers to a receptor that, in response to its cognate ligand, transduces a signal that inhibits a cellular response of an immune cell expressing the inhibitory receptor. Depending on the specific activator and inhibitory receptor pair, inhibitory receptors may inhibit a cellular response of an immune cell expressing both an activator and inhibitory receptor, even in the presence of activator ligand and an activator signal transduced by the activator receptor.

The term “chimeric antigen receptors” or “CARs” as used herein, refers to engineered receptors that graft an artificial specificity onto a particular immune effector cell, such as a helper T cell (CD4+), cytotoxic T cell (CD8+) or NK cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a particular antigen, e.g. HLA-A*11. Depending on the particular architecture of the CAR, and the intracellular signaling domains employed, the CAR may be an activator receptor, or an inhibitory receptor.

As used herein, “immune effector cell” refers to a cell that has differentiated into a form capable of modulating or effecting a specific immune response. This includes, without limitation, T cells, NK cells, B cells and macrophages, all of which are envisaged as within the scope of the instant disclosure. T cells include, but are not limited to, T lymphocytes, cytotoxic T cells, regulatory T cells, NKT cells, helper T cells and memory T cells.

In some embodiments, CARs comprise an intracellular signaling domain, a transmembrane domain, and an extracellular domain comprising an antigen binding domain as described herein. In some embodiments, CARs comprise fusions of single-chain variable fragments (scFvs) or scFabs derived from monoclonal antibodies, fused to a transmembrane domain and intracellular signaling domain. Either heavy-light (H-L) and light-heavy (L-H) scFvs may be used. In some embodiments, the transmembrane is isolated or derived from CD8a, CD28 or CD3 zeta.

In some embodiments, the CAR is an activator receptor, i.e. provides signal that activates an immune cell expressing the CAR. In some embodiments, the CAR comprises a CD3 zeta intracellular domain. In some embodiments, the CAR comprises one or more intracellular domains for additional co-stimulatory signaling, such as ICOS, CD137 (4-1BB), CD27, CD28, CD134, CD152 (CTLA-4), CD223 (LAG4), DAP10, and/or OX-40. In some embodiments, the CAR further comprises an extracellular hinge region. In some embodiments, the hinge is isolated or derived from IgG1, IgG4, CD8a or CD28.

In some embodiments, molecules can be co-expressed with the CAR, including co-stimulatory molecules, reporter genes for imaging (e.g., for positron emission tomography), gene products that conditionally ablate the T cells upon addition of a pro-drug, homing receptors, cytokines, and cytokine receptors. As used herein, characteristics attributed to a chimeric antigen receptor may be understood to refer to the receptor itself or to a host cell comprising the receptor.

As used herein, a “T cell receptor (TCR)” or “engineered TCR” refers to a protein complex found on the surface of T cells that is responsible for recognizing antigens bound to MHC molecules. TCRs comprise TCRα and TCRβ subunits, as well as CD3ε, CD3δ, CD3γ and CD3ζ subunits. The variable regions of the TCRα and TCRβ subunits form the extracellular antigen binding region of the TCR. TCRs can be engineered to target specific antigens, such as HLA-A*11, by grafting an extracellular antigen domain onto the TCR. In some cases, engineered TCRs may comprise one or more exogenous intracellular domains, for example the co-stimulatory domains described supra, or inhibitory intracellular domains. Thus, an engineered TCR of the disclosure may be an activator TCR, or an inhibitory TCR.

As used herein, an “epitope” or “antigenic determinant” refers to particular chemical groups or contiguous or non-contiguous peptide sequences on a molecule that are antigenic, i.e., that elicit a specific immune response. An antibody binds a particular antigenic epitope. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

The polypeptides described herein may be “isolated” polypeptides, meaning that the removed from living host. Isolated polypeptide may be expressed in a host cell, such as an immune cell, as a receptor and be displayed on the surface of that cell. The disclosure further provided soluble embodiments, include monoclonal antibodies, single-chain fragments (scFvs), bispecific or multispecific antibodies, and the like. It will be readily understood that the complementarity determining regions (CDRs) of antibodies can be grafted onto various scaffolds including antibody-based scaffolds, designed protein scaffolds, and non-protein scaffolds. The polypeptides of the disclosure may also be expressed in vivo (that is, in a subject organism, e.g., a human subject) rather than in vitro, such as by administration to the subject of a vector comprising a polynucleotide encoding the polypeptide.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical”, percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences of the present disclosure.

The term “human antibody” as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis or by somatic mutation in vivo). However, the term “human antibody” as used herein, is not intended to include antibodies in which CDR sequences derived from the germline of another mammalian species, such as a rabbit, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, VL, VH) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, “HLA-A*11 antibody” and “anti-HLA-A*11 antibody” are used interchangeably, and refer to an antibody that specifically binds to an HLA-A*11 polypeptide. Similarly, the term “HLA-A*11”, as mentioned in “HLA-A*11 binding”, “HLA-A*11 targeting”, “HLA-A*11 specific”, “HLA-A*11 expression” or other similar terms here, refers to HLA-A*11 polypeptide. HLA-A*11 antibodies of the disclosure bind to proteins falling within the HLA-A*11 allele group (for example HLA-A*11:01, HLA-A*11:02 and the like), and may not bind, or bind with lower affinity, to proteins falling within other HLA-A allele groups (for example, HLA-A*2 and the like). The person of ordinary skill in the art will recognize that some cross-reactivity with other HLA antigens may exist, but that the HLA-A*11 antibodies of the disclosure will still be considered to be specific to HLA-A*11. In some cases, specificity is considered in the context of the subject to be treated with an HLA-A*11 antibody or receptor of the disclosure. When the subject has both an HLA-A*11 allele and a second HLA-A allele not recognized, or only poorly recognized, by the HLA-A*11 antibody or receptor comprising an equivalent antigen binding domain, the HLA-A*11 antibody is specific to the HLA-A*11 allele of the subject.

As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this disclosure that is a polynucleotide encompasses both the double-stranded form and each of two complementary single-stranded forms known or predicted to make up the double-stranded form. A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this disclosure, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Preferred are compounds that are potent and can be administered locally at very low doses, thus minimizing systemic adverse effects.

### Anti-HLA-A*11 Polypeptides

The disclosure provides polypeptides comprising antigen binding domains that specifically bind MHC I complexes comprising an HLA-A α from A*11 alleles of HLA-A (HLA-A*11, encoding α¹¹). The antigen binding domains of the disclosure can be antibodies, or antigen-binding fragments or derivatives thereof, described below. The disclosure further provides antibodies and receptors comprising the polypeptides described herein, and cells and compositions comprising same.

HLA-A is a highly polymorphic group of human leukocyte antigens (HLA) of serotype A that are coded for by the HLA-A locus. There are currently over 6,425 HLA-A alleles known, which encode at least 3,929 HLA-A proteins (hla.alleles.org/nomenclature/stats.html). HLA-A*11 alleles are a group of HLA-A alleles. The A11 serotype is determined by antibody recognition of the α¹¹ subset of HLA-A α chains. There are currently over 40 recognized HLA-A*11 alleles, including HLA-A*11:01, HLA-A*11:02, HLA-A*11:03, and HLA-A*11:04, all of which are envisaged as within the scope of the instant invention.

HLA-A*11 binding polypeptides of the disclosure specifically bind to HLA-A*11 HLA-A alleles. In some embodiments, HLA-A*11 binding polypeptides specific to HLA-A*11 bind to HLA-A α¹¹ proteins produced by HLA-A*11 alleles and do not bind to HLA-A a chain produced by other HLA-A alleles, or HLA-A α chain produced by other HLA-A alleles with a lower affinity than they bind to HLA-A α¹¹. For example HLA-A*11 binding polypeptides may not bind to, or bind with lesser affinity, HLA-A α chain produced by e.g., HLA-A*01, HLA-A*02, or HLA-A*03.

An exemplary HLA-A*11α sequence, HLA-A*11:01:01, is provided as:

| (SEQ ID NO: 41) | |
|---|---|
| 1 | MAVMAPRTLL LLLSGALALT QTWAGSHSMR YFYTSVSRPG RGEPRFIAVG YVDDTQFVRF |
| 61 | DSDAASQRME PRAPWIEQEG PEYWDQETRN VKAQSQTDRV DLGTLRGYYN QSEDGSHTIQ |
| 121 | IMYGCDVGPD GRFLRGYRQD AYDGKDYIAL NEDLRSWTAA DMAAQITKRK WEAAHAAEQQ |
| 181 | RAYLEGRCVE WLRRYLENGK ETLQRTDPPK THMTHHPISD HEATLRCWAL GFYPAEITLT |
| 241 | WQRDGEDQTQ DTELVETRPA GDGTFQKWAA VVVPSGEEQR YTCHVQHEGL PKPLTLRWEL |
| 301 | SSQPTIPIVG IIAGLVLLGA VITGAVVAAV MWRRKSSDRK GGSYTQAASS DSAQGSDVSL |
| 361 | TACKV. |

The skilled artisan will appreciate that, due to the high degree of polymorphism, additional HLA-A*11 protein sequences may include one or more amino acid substitutions relative to SEQ ID NO: 41, and will still be considered proteins encoded HLA-A*11 alleles.

In the present disclosure, in vitro V(D)J recombination was employed to isolate and engineer scFv capable of binding MHC I comprising HLA-A α¹¹ with high selectivity (an HLA-A*11 antigen binding domain). These antibodies, and antigen binding domains derived therefrom, are described in more detail below.

The general structure of antibodies is known in the art. Briefly, an immunoglobulin monomer comprises two heavy chains and two light chains connected by disulfide bonds. Each heavy chain is paired with one of the light chains to which it is directly bound via a disulfide bond. Each heavy chain comprises a constant region (which varies depending on the isotype of the antibody) and a variable region. The variable region comprises three hypervariable regions (or complementarity determining regions) which are designated CDRH1, CDRH2 and CDRH3 and which are supported within framework regions. Each light chain comprises a constant region and a variable region, with the variable region comprising three hypervariable regions (designated CDRL1, CDRL2 and CDRL3) supported by framework regions in an analogous manner to the variable region of the heavy chain.

The hypervariable regions of each pair of heavy and light chains mutually cooperate to provide an antigen binding site that is capable of binding a target antigen. The binding specificity of a pair of heavy and light chains is defined by the sequence of CDR1, CDR2 and CDR3 of the heavy and light chains. Thus once a set of CDR sequences (i.e. the sequence of CDR1, CDR2 and CDR3 for the heavy and light chains) is determined which gives rise to a particular binding specificity, the set of CDR sequences can, in principle, be inserted into the appropriate positions within any other antibody framework regions linked with any antibody constant regions in order to provide a different antibody with the same antigen binding specificity.

In some embodiments, the polypeptides comprising HLA-A*11 antigen binding domains comprises one or more complementarity determining regions (CDRs) selected from the group disclosed in Tables 1 and 2 below.

In some embodiments, the HLA-A*11 antigen binding domain comprises a heavy chain (HC) comprising more CDRs selected from the group consisting of SEQ ID NOS: 1-19 set forth in Table 1 and Table 2.

In some embodiments, the antigen binding domain comprises the antigen binding domain comprises (a) a heavy chain (HC) complementarity determining region 1 (CDR1) sequence selected from the group consisting of SGGYYWS (SEQ ID NO: 1), TSGVGVG (SEQ ID NO: 2), SYAMH (SEQ ID NO: 3), SYDMH (SEQ ID NO: 4), and SYWMH (SEQ ID NO: 5); (b) a HC CDR2 sequence selected from the group consisting of YIYYSGSTYYNPSLKS (SEQ ID NO: 6), LIYWNDDKRYSPSLKS (SEQ ID NO: 7), WINAGNGNTKYSQKFQG (SEQ ID NO: 8), AIGTAGDTYYPGSVKG (SEQ ID NO: 9), and RINSDGSSTSYADSVKG (SEQ ID NO: 10); and (c) a HC CDR3 sequence selected from the group consisting of HYYYYSMDV (SEQ ID NO: 11), HYYYYYLDV (SEQ ID NO: 12), HYYYYMDV (SEQ ID NO: 13), HYYYYYMDV (SEQ ID NO: 14), KTTSFYFDY (SEQ ID NO: 15), RHMRLSCFDY (SEQ ID NO: 16), EGNGANPDAFDI (SEQ ID NO: 17), DLPGSYWYFDL (SEQ ID NO: 18), and GVLLYNWFDP (SEQ ID NO: 19).

In some embodiments, the HLA-A*11 antigen binding domain comprises a heavy chain. In some embodiments the HC comprises a CDR1 sequence of SGGYYWS (SEQ ID NO: 1), a CDR2 sequence of YIYYSGSTYYNPSLKS (SEQ ID NO: 6), and a CDR3 sequence of HYYYYYMDV (SEQ ID NO: 14). In some embodiments, the HC comprises a CDR1 sequence of TSGVGVG (SEQ ID NO: 2), a CDR2 sequence of LIYWNDDKRYSPSLKS (SEQ ID NO: 7), and a CDR3 sequence of KTTSFYFDY (SEQ ID NO: 15). In some embodiments, the HC comprises a CDR1 sequence of SGGYYWS (SEQ ID NO: 1), a CDR2 sequence of YIYYSGSTYYNPSLKS (SEQ ID NO: 6), and a CDR3 sequence of HYYYYMDV (SEQ ID NO: 13). In some embodiments, the HC comprises a CDR1 sequence of SYWMH (SEQ ID NO: 5), a CDR2 sequence of RINSDGSSTSYADSVKG (SEQ ID NO: 10), and a CDR3 sequence of GVLLYNWFDP (SEQ ID NO: 19). In some embodiments, the HC comprises a CDR1 sequence of SGGYYWS (SEQ ID NO: 1), a CDR2 sequence of YIYYSGSTYYNPSLKS (SEQ ID NO: 6) and a CDR3 sequence of HYYYYYLDV (SEQ ID NO: 12). In some embodiments, the HC comprises a CDR1 sequence of SYDMH (SEQ ID NO: 4), a CDR2 sequence of AIGTAGDTYYPGSVKG (SEQ ID NO: 9), and a CDR3 sequence of HYYYYYLDV (SEQ ID NO: 12). In some embodiments, the HC comprises a CDR1 sequence of SYDMH (SEQ ID NO: 4), a CDR2 sequence of AIGTAGDTYYPGSVKG (SEQ ID NO: 9), and a CDR3 sequence of DLPGSYWYFDL (SEQ ID NO: 18). In some embodiments, the HC comprises a CDR1 sequence of SYAMH (SEQ ID NO: 3), a CDR2 sequence of WINAGNGNTKYSQKFQG (SEQ ID NO: 8) and a CDR3 sequence of EGNGANPDAFDI (SEQ ID NO: 17). In some embodiments, the HC comprises a CDR1 sequence of TSGVGVG (SEQ ID NO: 2), a CDR2 sequence of LIYWNDDKRYSPSLKS (SEQ ID NO: 7), and a CDR3 sequence of RHMRLSCFDY (SEQ ID NO: 16). In some embodiments, the HC comprises a CDR1 sequence of SGGYYWS (SEQ ID NO: 1), a CDR2 sequence of YIYYSGSTYYNPSLKS (SEQ ID NO: 6), and a CDR3 sequence of HYYYYSMDV (SEQ ID NO: 11). In some embodiments, the HLA-A*11 antigen binding domain further comprises a light chain.

In some embodiments, the HLA-A*11 antigen binding domain comprises a light chain. In some embodiments the LC comprises a light chain (LC) complementarity determining region 1 (CDR1) comprising a sequence of RASQSISSYLN (SEQ ID NO: 20), a LC CDR2 comprising a sequence of AASSLQS (SEQ ID NO: 21) and a LC CDR3 comprising a sequence of QQSYSTPLT (SEQ ID NO: 22).

In some embodiments, the HLA-A*11 antigen binding domain comprises a HC CDR1 comprising TSGVGVG (SEQ ID NO: 2), a HC CDR2 comprising LIYWNDDKRYSPSLKS (SEQ ID NO: 7), a HC CDR3 comprising KTTSFYFDY (SEQ ID NO: 15), a LC CDR1 comprising RASQSISSYLN (SEQ ID NO: 20), a LC CDR2 comprising AASSLQS (SEQ ID NO: 21), and a LC CDR3 comprising QQSYSTPLT (SEQ ID NO: 22).

In some embodiments, the HLA-A*11 antigen binding domain comprises a HC CDR1 comprising SYWMH (SEQ ID NO: 5), a HC CDR2 comprising RINSDGSSTSYADSVKG (SEQ ID NO: 10), a HC CDR comprising GVLLYNWFDP (SEQ ID NO: 19), a LC CDR1 comprising RASQSISSYLN (SEQ ID NO: 20), a LC CDR2 comprising AASSLQS (SEQ ID NO: 21), and a LC CDR3 comprising QQSYSTPLT (SEQ ID NO: 22).

TABLE 1
Heavy Chain CDR sequences for exemplary HLA-A*11 antigen binding domains.

| scFv | HC CDR1 | HC CDR2 | HC CDR3 |
|---|---|---|---|
| 1 | SGGYYWS | YIYYSGSTYYNPSLKS (SEQ ID | HYYYYYMDV |
| | (SEQ ID NO: 1) | NO: 6) | (SEQ ID NO: 14) |
| 2 | TSGVGVG | LIYWNDDKRYSPSLKS (SEQ ID | KTTSFYFDY |
| | (SEQ ID NO: 2) | NO: 7) | (SEQ ID NO: 15) |
| 3 | SGGYYWS | YIYYSGSTYYNPSLKS (SEQ ID | HYYYYMDV |
| | (SEQ ID NO: 1) | NO: 6) | (SEQ ID NO: 13) |
| 4 | SYWMH | RINSDGSSTSYADSVKG | GVLLYNWFDP |
| | (SEQ ID NO: 5) | (SEQ ID NO: 10) | (SEQ ID NO: 19) |
| 5 | SGGYYWS | YIYYSGSTYYNPSLKS (SEQ ID | HYYYYYLDV |
| | (SEQ ID NO: 1) | NO: 6) | (SEQ ID NO: 12) |
| 6 | SYDMH | AIGTAGDTYYPGSVKG (SEQ ID | DLPGSYWYFDL |
| | (SEQ ID NO: 4) | NO: 9) | (SEQ ID NO: 18) |
| 7 | SYAMH | WINAGNGNTKYSQKFQG | EGNGANPDAFDI |
| | (SEQ ID NO: 3) | (SEQ ID NO: 8) | (SEQ ID NO: 17) |
| 8 | TSGVGVG | LIYWNDDKRYSPSLKS (SEQ ID | RHMRLSCFDY |
| | (SEQ ID NO: 2) | NO: 7) | (SEQ ID NO: 16) |
| 9 | SGGYYWS | YIYYSGSTYYNPSLKS (SEQ ID | HYYYYSMDV |
| | (SEQ ID NO: 1) | NO: 6) | (SEQ ID NO: 11) |

TABLE 2
Light Chain CDR sequences for exemplary HLA-A*11 antigen binding domains

| scFv | LC CDR1 | LC CDR2 | LC CDR3 |
|---|---|---|---|
| 1-9 | RASQSISSYLN | AASSLQS | QQSYSTPLT |
| | (SEQ ID NO: 20) | (SEQ ID NO: 21) | (SEQ ID NO: 22) |

In some embodiments, the HLA-A*11 antigen binding domain comprises a heavy chain and a light chain. Exemplary variable heavy and light chain sequences are provided in Table 3, below.

In some embodiments, the antigen binding domain comprises a variable heavy chain comprising a sequence of SEQ ID NOS: 42-50, or a sequence having at least 90%, at least 95%, at least 97%, at least 98% or at least 99% identity thereto. In some embodiments, the antigen binding domain comprises a variable heavy chain comprising a sequence of SEQ ID NO: 47, or a sequence having at least 90%, at least 95%, at least 97%, at least 98% or at least 99% identity thereto. In some embodiments, the antigen binding domain comprises a variable heavy chain comprising a sequence of SEQ ID NO: 49, or a sequence having at least 90%, at least 95%, at least 97%, at least 98% or at least 99% identity thereto. In some embodiments, the antigen binding domain comprises a variable heavy chain comprising a sequence of SEQ ID NOS: 42-50. In some embodiments, the antigen binding domain comprises a variable heavy chain comprising a sequence of SEQ ID NO: 47. In some embodiments, the antigen binding domain comprises a variable heavy chain comprising a sequence of SEQ ID NO: 49.

In some embodiments, the antigen binding domain comprises a variable light chain comprising a sequence of SEQ ID NO: 51, or a sequence having at least 90%, at least 95%, at least 97%, at least 98% or at least 99% identity thereto. In some embodiments, the antigen binding domain comprises a variable light chain comprising a sequence of SEQ ID NO: 51.

In some embodiments, the antigen binding domain comprises a variable heavy chain and a variable light chain. In some embodiments, the variable heavy chain comprises a sequence of SEQ ID NOS: 42-50, and the variable light chain comprises a sequence of SEQ ID NO: 51. In some embodiments, the variable heavy chain comprises a sequence of SEQ ID NO: 47, and the variable light chain comprises a sequence of SEQ ID NO: 51. In some embodiments, the variable heavy chain comprises a sequence of SEQ ID NO: 49, and the variable light chain comprises a sequence of SEQ ID NO: 51.

TABLE 3
Exemplary heavy and light chain sequences

| Heavy | | |
|---|---|---|
| Chain | | |
| Sequences | Protein Sequence | DNA Sequence |
| 9 | QVQLQESGPGL | CAGGTGCAGCTGCAGGAAAGCGGCCCTGGCCT |
| | VKPSQTLSLTCT | GGTGAAACCCAGCCAGACCCTGAGCCTGACCT |
| | VSGGSISSGGYY | GCACAGTGTCCGGCGGCTCGATCAGCAGCGGC |
| | WSWIRQPPGKG | GGCTACTACTGGTCCTGGATCAGACAGCCCCC |
| | LEWIGYIYYSGS | TGGCAAGGGCCTGGAATGGATCGGCTACATCT |
| | TYYNPSLKSRV | ACTACAGCGGCAGCACCTACTACAACCCCAGC |
| | TISVDTSKNQFS | CTGAAGTCCAGAGTGACCATCAGCGTGGACAC |
| | LKLSSVTAADT | CAGCAAGAACCAGTTCAGCCTGAAGCTGAGCA |
| | AVYYCARHYY | GCGTGACAGCCGCCGACACCGCTGTGTATTAC |
| | YYSMDVWGKG | TGTGCGAGACACTACTACTACTACTCCATGGA |
| | TTVTVSS (SEQ | CGTCTGGGGCAAAGGGACCACGGTCACCGTGT |
| | ID NO: 42) | CCTCA (SEQ ID NO: 52) |
| 8 | QITLKESGPTLV | CAGATCACCCTGAAAGAGTCCGGCCCCACCCT |
| | KPTQTLTLTCTF | GGTGAAACCCACCCAGACCCTGACCCTGACAT |
| | SGFSLSTSGVGV | GCACCTTCAGCGGCTTCAGCCTGAGCACCTCT |
| | GWIRQPPGKAL | GGCGTGGGCGTGGGCTGGATCAGACAGCCTCC |
| | EWLALIYWNDD | CGGCAAGGCCCTGGAATGGCTGGCCCTGATCT |
| | KRYSPSLKSRLT | ACTGGAACGACGACAAGCGGTACAGCCCCAGC |
| | ITKDTSKNQVV | CTGAAGTCCCGGCTGACCATCACCAAGGACAC |
| | LTMTNMDPVDT | CTCGAAGAACCAGGTGGTGCTGACCATGACAA |
| | ATYYCAHRHM | ACATGGACCCCGTGGACACCGCCACATATTAC |
| | RLSCFDYWGQG | TGTGCACACAGACACATGCGTTTAAGCTGTTTT |
| | TLVTVSS (SEQ | GACTACTGGGGCCAGGGAACCCTGGTCACCGT |
| | ID NO: 43) | GTCCTCA (SEQ ID NO: 53) |
| 7 | QVQLVQSGAEV | CAGGTGCAGCTGGTGCAGTCTGGCGCCGAAGT |
| | KKPGASVKVSC | GAAGAAACCTGGCGCCTCCGTGAAGGTGTCCT |
| | KASGYTFTSYA | GCAAGGCCAGCGGCTACACCTTCACCAGCTAC |
| | MHWVRQAPGQ | GCCATGCACTGGGTTCGACAGGCCCCTGGCCA |
| | RLEWMGWINA | GAGACTGGAATGGATGGGCTGGATCAACGCCG |
| | GNGNTKYSQKF | GCAACGGCAACACCAAGTACAGCCAGAAATTC |
| | QGRVTITRDTSA | CAGGGCAGAGTGACCATCACCCGGGACACCAG |
| | STAYMELSSLRS | CGCCAGCACCGCCTACATGGAACTGAGCAGCC |
| | EDTAVYYCARE | TGCGGAGCGAGGACACCGCTGTGTATTACTGT |
| | GNGANPDAFDI | GCGAGAGAAGGAAATGGTGCCAACCCTGATGC |
| | WGQGTMVTVS | TTTTGATATCTGGGGCCAAGGGACAATGGTCA |
| | S (SEQ ID NO: | CCGTGTCCTCA (SEQ ID NO: 54) |
| | 44) | |
| 6 | EVQLVESGGGL | GAAGTGCAGCTGGTGGAAAGCGGCGGAGGCC |
| | VQPGGSLRLSC | TGGTGCAGCCTGGCGGCAGCCTGAGACTGTCT |
| | AASGFTFSSYD | TGCGCCGCCAGCGGCTTCACCTTCAGCAGCTA |
| | MHWVRQATGK | CGACATGCACTGGGTCCGCCAGGCCACCGGCA |
| | GLEWVSAIGTA | AGGGACTGGAATGGGTGTCCGCCATCGGCACA |
| | GDTYYPGSVKG | GCCGGCGACACTTACTACCCCGGCAGCGTGAA |
| | RFTISRENAKNS | GGGCCGGTTCACCATCAGCAGAGAGAACGCCA |
| | LYLQMNSLRAG | AGAACAGCCTGTACCTGCAGATGAACAGCCTT |
| | DTAVYYCARDL | CGAGCCGGCGATACCGCCGTGTATTACTGTGC |
| | PGSYWYFDLW | AAGAGATCTCCCTGGTAGCTACTGGTACTTCG |
| | GRGTLVTVSS | ATCTCTGGGGCCGTGGCACCCTGGTCACTGTGT |
| | (SEQ ID NO: 45) | CCTCA (SEQ ID NO: 55) |
| 5 | QVQLQESGPGL | CAGGTGCAGCTGCAGGAAAGCGGCCCTGGCCT |
| | VKPSQTLSLTCT | GGTGAAACCCAGCCAGACCCTGAGCCTGACCT |
| | VSGGSISSGGYY | GCACAGTGTCCGGCGGCTCGATCAGCAGCGGC |
| | WSWIRQPPGKG | GGCTACTACTGGTCCTGGATCAGACAGCCCCC |
| | LEWIGYIYYSGS | TGGCAAGGGCCTGGAATGGATCGGCTACATCT |
| | TYYNPSLKSRV | ACTACAGCGGCAGCACCTACTACAACCCCAGC |
| | TISVDTSKNQFS | CTGAAGTCCAGAGTGACCATCAGCGTGGACAC |
| | LKLSSVTAADT | CAGCAAGAACCAGTTCAGCCTGAAGCTGAGCA |
| | AVYYCARHYY | GCGTGACAGCCGCCGACACCGCTGTGTATTAC |
| | YYYLDVWGKG | TGTGCGAGACACTACTACTACTACTACCTGGA |
| | TTVTVSS (SEQ | CGTCTGGGGCAAAGGGACCACGGTCACCGTGT |
| | ID NO: 46) | CCTCA (SEQ ID NO: 56) |
| 4 | EVQLVESGGGL | GAAGTGCAGCTGGTGGAAAGCGGCGGAGGCC |
| | VQPGGSLRLSC | TGGTGCAGCCTGGCGGCAGCCTGAGACTGTCT |
| | AASGFTFSSYW | TGCGCCGCCAGCGGCTTCACCTTCAGCAGCTA |
| | MHWVRQAPGK | CTGGATGCACTGGGTCCGCCAGGCCCCTGGCA |
| | GLVWVSRINSD | AGGGACTGGTCTGGGTGTCTCGAATCAACAGC |
| | GSSTSYADSVK | GACGGCAGCAGCACCAGCTACGCCGACAGCGT |
| | GRFTISRDNAKN | GAAGGGCCGGTTCACCATCAGCCGGGACAACG |
| | TLYLQMNSLRA | CCAAGAACACCCTGTACCTGCAGATGAACAGC |
| | EDTAVYYCCLG | CTGCGGGCCGAGGACACCGCCGTGTATTACTG |
| | VLLYNWFDPW | TTGTTTGGGTGTTTTATTATACAACTGGTTCGA |
| | GQGTLVTVSS | CCCCTGGGGCCAGGGAACCCTGGTCACCGTGT |
| | (SEQ ID NO: 47) | CCTCA (SEQ ID NO: 57) |
| 3 | QVQLQESGPGL | CAGGTGCAGCTGCAGGAAAGCGGCCCTGGCCT |
| | VKPSQTLSLTCT | GGTGAAACCCAGCCAGACCCTGAGCCTGACCT |
| | VSGGSISSGGYY | GCACAGTGTCCGGCGGCTCGATCAGCAGCGGC |
| | WSWIRQPPGKG | GGCTACTACTGGTCCTGGATCAGACAGCCCCC |
| | LEWIGYIYYSGS | TGGCAAGGGCCTGGAATGGATCGGCTACATCT |
| | TYYNPSLKSRV | ACTACAGCGGCAGCACCTACTACAACCCCAGC |
| | TISVDTSKNQFS | CTGAAGTCCAGAGTGACCATCAGCGTGGACAC |
| | LKLSSVTAADT | CAGCAAGAACCAGTTCAGCCTGAAGCTGAGCA |
| | AVYYCARHYY | GCGTGACAGCCGCCGACACCGCTGTGTATTAC |
| | YYMDVWGKGT | TGTGCGAGACACTACTACTACTACATGGACGT |
| | TVTVSS (SEQ ID | CTGGGGCAAAGGGACCACGGTCACCGTGTCCT |
| | NO: 48) | CA (SEQ ID NO: 58) |
| 2 | QITLKESGPTLV | CAGATCACCCTGAAAGAGTCCGGCCCCACCCT |
| | KPTQTLTLTCTF | GGTGAAACCCACCCAGACCCTGACCCTGACAT |
| | SGFSLSTSGVGV | GCACCTTCAGCGGCTTCAGCCTGAGCACCTCT |
| | GWIRQPPGKAL | GGCGTGGGCGTGGGCTGGATCAGACAGCCTCC |
| | EWLALIYWNDD | CGGCAAGGCCCTGGAATGGCTGGCCCTGATCT |
| | KRYSPSLKSRLT | ACTGGAACGACGACAAGCGGTACAGCCCCAGC |
| | ITKDTSKNQVV | CTGAAGTCCCGGCTGACCATCACCAAGGACAC |
| | LTMTNMDPVDT | CTCGAAGAACCAGGTGGTGCTGACCATGACAA |
| | ATYYCAHKTTS | ACATGGACCCCGTGGACACCGCCACATATTAC |
| | FYFDYWGQGTL | TGTGCACACAAAACGACGTCGTTTTACTTTGAC |
| | VTVSS (SEQ ID | TACTGGGGCCAGGGAACCCTGGTCACCGTGTC |
| | NO: 49) | CTCA (SEQ ID NO: 59) |
| 1 | QVQLQESGPGL | CAGGTGCAGCTGCAGGAAAGCGGCCCTGGCCT |
| | VKPSQTLSLTCT | GGTGAAACCCAGCCAGACCCTGAGCCTGACCT |
| | VSGGSISSGGYY | GCACAGTGTCCGGCGGCTCGATCAGCAGCGGC |
| | WSWIRQPPGKG | GGCTACTACTGGTCCTGGATCAGACAGCCCCC |
| | LEWIGYIYYSGS | TGGCAAGGGCCTGGAATGGATCGGCTACATCT |
| | TYYNPSLKSRV | ACTACAGCGGCAGCACCTACTACAACCCCAGC |
| | TISVDTSKNQFS | CTGAAGTCCAGAGTGACCATCAGCGTGGACAC |
| | LKLSSVTAADT | CAGCAAGAACCAGTTCAGCCTGAAGCTGAGCA |
| | AVYYCARHYY | GCGTGACAGCCGCCGACACCGCTGTGTATTAC |
| | YYYMDVWGKG | TGTGCGAGACACTACTACTACTACTACATGGA |
| | TTVTVSS (SEQ | CGTCTGGGGCAAAGGGACCACGGTCACCGTGT |
| | ID NO: 50) | CCTCA (SEQ ID NO: 60) |
| Light | DIQMTQSPSSLS | GACATCCAGATGACCCAGTCTCCATCCTCCCTG |
| Chain | ASVGDRVTITCR | TCTGCATCTGTAGGAGACAGAGTCACCATCAC |
| Sequence | ASQSISSYLNWY | TTGCCGGGCAAGTCAGAGCATTAGCAGCTATT |
| | QQKPGKAPKLLI | TAAATTGGTATCAGCAGAAACCAGGGAAAGCC |
| | YAASSLQSGVPS | CCTAAGCTCCTGATCTATGCTGCATCCAGTTTG |
| | RFSGSGSGTDFT | CAAAGTGGGGTCCCATCAAGGTTCAGTGGCAG |
| | LTISSLQPEDFA | TGGATCTGGGACAGATTTCACTCTCACCATCA |
| | TYYCQQSYSTP | GCAGTCTGCAACCTGAAGATTTTGCAACTTACT |
| | LTFGGGTKVEIK | ACTGTCAACAGAGTTACAGTACCCCTCTCACTT |
| | (SEQ ID NO: 51) | TCGGCGGCGGAACAAAGGTGGAGATCAAG |
| | | (SEQ ID NO: 61) |

In some embodiments, provided herein is an antibody or antigen binding fragment thereof comprising an antigen binding domain that specifically binds to MHC I comprising an α¹¹ chain encoded by an HLA-A*11 allele, as described herein. In some embodiments, the antibody is a monoclonal antibody. In some embodiments, the monoclonal antibody is a fully human antibody. In some embodiments, the antibody is a humanized antibody. In some embodiments, the antibody is a chimeric antibody. In other embodiments, the antibody is an immunoglobulin molecule. In particular examples, the antibody is an IgG.

Anti-HLA-A*11 antibodies of the disclosure can be monospecific—i.e., contain a single antigen binding domain specific to HLA-A*11. Alternatively, anti-HLA-A*11 antibodies of the disclosure can be multispecific, such as bispecific antibodies. Various formats of multispecific antibodies will be known to persons of ordinary skill in the art, and are envisaged as within the scope of the instant disclosure.

In some embodiments, the antigen binding domain is an antibody fragment, such as a Fab fragment, a Fab′ fragment, a F(ab)′₂ fragment, a single chain variable fragment (scFv), or a disulfide stabilized variable fragment (dsFv).

The monoclonal antibodies disclosed herein can be of any isotype. The monoclonal antibody can be, for example, an IgM or an IgG antibody, such as IgG1 or an IgG2. The class of an antibody that specifically binds MAGE-A3 can be switched with another (for example, IgG can be switched to IgM), according to well-known procedures. Class switching can also be used to convert one IgG subclass to another, such as from IgG1 to IgG2.

Antibody fragments comprising HLA-A*11 antigen binding domains are also encompassed by the present disclosure, such as single-domain antibodies (e.g., VH domain antibodies), Fab, F(ab′)₂, and Fv. These antibody fragments retain the ability to selectively bind with the antigen. These fragments include:
(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule can be obtained by treating whole antibody with pepsin, followed by reduction, to yield an intact light chain and a portion of the heavy chain; two Fab′ fragments are obtained per antibody molecule;
(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;
(4) Fv, a genetically engineered fragment containing the variable region of the light chain and the variable region of the heavy chain expressed as two chains; (5) Single chain antibody (such as scFv), a genetically engineered molecule containing the variable region of the light chain, the variable region of the heavy chain, linked by a suitable polypeptide linker as a genetically fused single chain molecule;
(6) A dimer of a single chain antibody (scFv₂), defined as a dimer of a scFv (also known as a “miniantibody”);
(7) VH single-domain antibody, an antibody fragment consisting of a heavy chain variable domain; and
(8) A single chain Fab fragment (scFab), which can be formed by the introduction of a polypeptide linker between the Fd and the light chain to result in the formation of a single chain Fab fragment.

Methods of making these fragments are known in the art (see for example, Harlow and Lane, Antibodies: A Laboratory Manual, Cold Spring Harbor Laboratory, New York, 1988).

In some cases, antibody fragments can be prepared by proteolytic hydrolysis of the antibody or by expression in a host cell (such as E. coli) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. Nos. 4,036,945 and 4,331,647).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

In some embodiments, the HLA-A*11 antigen binding domain is a scFv. The scFv can be in either orientation, i.e. a variable heavy chain (VH)-linker-variable light chain (VL) or a VL-linker-VH orientation.

In some embodiments, the variable heavy chain of the scFv comprises a sequence selected from the group consisting of SEQ ID NOS: 42-50, or a sequence having at least 90%, at least 95%, at least 97%, at least 98% or at least 99% identity thereto, and the variable light chain of the scFv, comprises a sequence of SEQ ID NO: 51, or a sequence having at least 90%, at least 95%, at least 97%, at least 98% or at least 99% identity thereto. In some embodiments, the variable heavy chain of the scFv comprises a sequence selected from the group consisting of SEQ ID NOS: 42-50, and the variable light chain of the scFv comprises a sequence of SEQ ID NO: 51. In some embodiments, the variable heavy chain of the scFv comprises a sequence selected from the group consisting of SEQ ID NOS: 47 and 49, and the variable light chain of the scFv comprises a sequence of SEQ ID NO: 51.

In some embodiments, the scFv comprises a sequence selected from the group set forth in Table 4, below. In some embodiments, the scFv comprises a sequence selected from the group consisting of SEQ ID NOS: 23-31, or a sequence having at least 90%, at least 95%, at least 97%, at least 98% or at least 99% identity thereto. In some embodiments, the scFv comprises a sequence selected from the group consisting of SEQ ID NOS: 23-31. In some embodiments, the scFv comprises a sequence of SEQ ID NO: 28 or SEQ ID NO: 30, or a sequence having at least 90%, at least 95%, at least 97%, at least 98% or at least 99% identity thereto. In some embodiments, the scFv comprises a sequence of SEQ ID NO: 28 or SEQ ID NO: 30.

TABLE 4
Exemplary HLA-A*11 scFv antigen binding domains.

| | | DNA |
|---|---|---|
| scFv | Protein Sequence | Sequence |
| 9 | QVQLQESGPGLVKPSQTLSLTCTVSGGSISSGGYYWSWIRQPPGKG | (SEQ ID |
| | LEWIGYIYYSGSTYYNPSLKSRVTISVDTSKNQFSLKLSSVTAADTA | NO: 32) |
| | VYYCARHYYYYSMDVWGKGTTVTVSSGGGGSGGGGSGGGGSGG | |
| | DIQMTQSPSSLSASVGDRVTITCRASQSISSYLNWYQQKPGKAPKLL | |
| | IYAASSLQSGVPSRFSGSGSGTDFTLTISSLQPEDFATYYCQQSYSTP | |
| | LTFGGGTKVEIK (SEQ ID NO: 23) | |
| 8 | QITLKESGPTLVKPTQTLTLTCTFSGFSLSTSGVGVGWIRQPPGKAL | (SEQ ID |
| | EWLALIYWNDDKRYSPSLKSRLTITKDTSKNQVVLTMTNMDPVDT | NO: 33) |
| | ATYYCAHRHMRLSCFDYWGQGTLVTVSSGGGGSGGGGSGGGGSG | |
| | GDIQMTQSPSSLSASVGDRVTITCRASQSISSYLNWYQQKPGKAPK | |
| | LLIYAASSLQSGVPSRFSGSGSGTDFTLTISSLQPEDFATYYCQQSYS | |
| | TPLTFGGGTKVEIK (SEQ ID NO: 24) | |
| 7 | QVQLVQSGAEVKKPGASVKVSCKASGYTFTSYAMHWVRQAPGQR | (SEQ ID |
| | LEWMGWINAGNGNTKYSQKFQGRVTITRDTSASTAYMELSSLRSE | NO: 34) |
| | DTAVYYCAREGNGANPDAFDIWGQGTMVTVSSGGGGSGGGGSGG | |
| | GGSGGDIQMTQSPSSLSASVGDRVTITCRASQSISSYLNWYQQKPG | |
| | KAPKLLIYAASSLQSGVPSRFSGSGSGTDFTLTISSLQPEDFATYYCQ | |
| | QSYSTPLTFGGGTKVEIK (SEQ ID NO: 25) | |
| 6 | EVQLVESGGGLVQPGGSLRLSCAASGFTFSSYDMHWVRQATGKGL | (SEQ ID |
| | EWVSAIGTAGDTYYPGSVKGRFTISRENAKNSLYLQMNSLRAGDT | NO: 35) |
| | AVYYCARDLPGSYWYFDLWGRGTLVTVSSGGGGSGGGGSGGGGS | |
| | GGDIQMTQSPSSLSASVGDRVTITCRASQSISSYLNWYQQKPGKAP | |
| | KLLIYAASSLQSGVPSRFSGSGSGTDFTLTISSLQPEDFATYYCQQSY | |
| | STPLTFGGGTKVEIK (SEQ ID NO: 26) | |
| 5 | QVQLQESGPGLVKPSQTLSLTCTVSGGSISSGGYYWSWIRQPPGKG | (SEQ ID |
| | LEWIGYIYYSGSTYYNPSLKSRVTISVDTSKNQFSLKLSSVTAADTA | NO: 36) |
| | VYYCARHYYYYYLDVWGKGTTVTVSSGGGGSGGGGSGGGGSGG | |
| | DIQMTQSPSSLSASVGDRVTITCRASQSISSYLNWYQQKPGKAPKLL | |
| | IYAASSLQSGVPSRFSGSGSGTDFTLTISSLQPEDFATYYCQQSYSTP | |
| | LTFGGGTKVEIK (SEQ ID NO: 27) | |
| 4 | EVQLVESGGGLVQPGGSLRLSCAASGFTFSSYWMHWVRQAPGKG | (SEQ ID |
| | LVWVSRINSDGSSTSYADSVKGRFTISRDNAKNTLYLQMNSLRAED | NO: 37) |
| | TAVYYCCLGVLLYNWFDPWGQGTLVTVSSGGGGSGGGGSGGGGS | |
| | GGDIQMTQSPSSLSASVGDRVTITCRASQSISSYLNWYQQKPGKAP | |
| | KLLIYAASSLQSGVPSRFSGSGSGTDFTLTISSLQPEDFATYYCQQSY | |
| | STPLTFGGGTKVEIK (SEQ ID NO: 28) | |
| 3 | QVQLQESGPGLVKPSQTLSLTCTVSGGSISSGGYYWSWIRQPPGKG | (SEQ ID |
| | LEWIGYIYYSGSTYYNPSLKSRVTISVDTSKNQFSLKLSSVTAADTA | NO: 38 |
| | VYYCARHYYYYMDVWGKGTTVTVSSGGGGSGGGGSGGGGSGGD | |
| | IQMTQSPSSLSASVGDRVTITCRASQSISSYLNWYQQKPGKAPKLLI | |
| | YAASSLQSGVPSRFSGSGSGTDFTLTISSLQPEDFATYYCQQSYSTPL | |
| | TFGGGTKVEIK (SEQ ID NO: 29) | |
| 2 | QITLKESGPTLVKPTQTLTLTCTFSGFSLSTSGVGVGWIRQPPGKAL | (SEQ ID |
| | EWLALIYWNDDKRYSPSLKSRLTITKDTSKNQVVLTMTNMDPVDT | NO: 39) |
| | ATYYCAHKTTSFYFDYWGQGTLVTVSSGGGGSGGGGSGGGGSGG | |
| | DIQMTQSPSSLSASVGDRVTITCRASQSISSYLNWYQQKPGKAPKLL | |
| | IYAASSLQSGVPSRFSGSGSGTDFTLTISSLQPEDFATYYCQQSYSTP | |
| | LTFGGGTKVEIK (SEQ ID NO: 30) | |
| 1 | QVQLQESGPGLVKPSQTLSLTCTVSGGSISSGGYYWSWIRQPPGKG | (SEQ ID |
| | LEWIGYIYYSGSTYYNPSLKSRVTISVDTSKNQFSLKLSSVTAADTA | NO: 40) |
| | VYYCARHYYYYYMDVWGKGTTVTVSSGGGGSGGGGSGGGGSGG | |
| | DIQMTQSPSSLSASVGDRVTITCRASQSISSYLNWYQQKPGKAPKLL | |
| | IYAASSLQSGVPSRFSGSGSGTDFTLTISSLQPEDFATYYCQQSYSTP | |
| | LTFGGGTKVEIK (SEQ ID NO: 31) | |

In Table 4, CDR sequences are underlined.

### Antibodies

The present disclosure provides anti-HLA-A*11 antibodies and antigen-binding fragments thereof.

The antibodies of the present disclosure can be purified to homogeneity. The separation and purification of the antibodies can be performed by employing conventional protein separation and purification methods.

By way of example only, the antibody can be separated and purified by appropriately selecting and combining use of chromatography columns, filters, ultrafiltration, salt precipitation, dialysis, preparative polyacrylamide gel electrophoresis, isoelectric focusing electrophoresis, and the like. Strategies for Protein Purification and Characterization: A Laboratory Course Manual. Daniel R. Marshak et al. eds., Cold Spring Harbor Laboratory Press (1996); Antibodies: A Laboratory Manual. Ed Harlow and David Lane, Cold Spring Harbor Laboratory (1988).

Non-limiting examples of chromatography include affinity chromatography, ion exchange chromatography, hydrophobic chromatography, gel filtration chromatography, reverse phase chromatography, and adsorption chromatography. In one aspect, chromatography can be performed by employing liquid chromatography such as HPLC or FPLC.

### Variations

In some embodiments, one or more amino acid residues in a CDR of the antigen binding domains provided herein are substituted with another amino acid. The substitution may be “conservative” in the sense of being a substitution within the same family of amino acids. The naturally occurring amino acids may be divided into the following four families and conservative substitutions will take place within those families: (1) amino acids with basic side chains: lysine, arginine, histidine; (2) amino acids with acidic side chains: aspartic acid, glutamic acid; (3) amino acids with uncharged polar side chains: asparagine, glutamine, serine, threonine, tyrosine; and (4) amino acids with nonpolar side chains: glycine, alanine, valine, leucine, isoleucine, proline, phenylalanine, methionine, tryptophan, cysteine.

In another aspect, one or more amino acid residues are added to or deleted from one or more CDRs of an antigen binding domain described herein. Such additions or deletions may occur at the N or C termini of the CDR or at a position within the CDR.

By varying the amino acid sequence of the CDRs of an antibody by addition, deletion or substitution of amino acids, various effects such as increased binding affinity for the target antigen may be obtained.

It is to be appreciated that antibodies of the disclosure comprising such varied CDR sequences may still bind HLA-A*11 with similar specificity and sensitivity profiles. This may be tested by way of the binding assays disclosed in the Examples and that are known in the art. When the antigen binding domain comprises an antibody or antibody fragment, the constant regions of antibodies may also be varied. For example, antibodies may be provided with Fc regions of any isotype: IgA (IgA1, IgA2), IgD, IgE, IgG (IgG1, IgG2, IgG3, IgG4) or IgM.

One of skill will realize that conservative variants of the antigen binding domains and antibodies can be produced. Such conservative variants employed in antibody fragments, such as dsFv fragments or in scFv fragments, will retain critical amino acid residues necessary for correct folding and stabilizing between the VH and the VL regions. In some embodiments, the variants will retain the charge characteristics of the residues, for example.

Amino acid substitutions (such as at most one, at most two, at most three, at most four, at most five, at most six, at most seven, at most eight, at most nine, at most ten, at most 11, at most 12, at most 13, at most 14, at most 15, at most 16, at most 17, at most 18, at most 19, or at most 20 amino acid substitutions) can be made in the VH and/or the VL regions to increase yield. Conservative amino acid substitution tables providing functionally similar amino acids are well known to one of ordinary skill in the art. The following six groups are examples of amino acids that are considered to be conservative substitutions for one another: (1) Alanine (A), Serine (S), Threonine (T); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W).

Amino acid substitutions, deletions, and additions, and other such sequence variations, may be performed based on sequence alignment techniques using existing sequence alignment tools.

### Modifications

The disclosure provides polypeptides comprising HLA-A*11 antigen binding domains and antibodies, comprising one or more modifications. Modifications include, inter alia detectable labels, conjugates to therapeutic agents and agents that increase stability or bioavailability, and multimerization domains.

As used herein, the term “detectable label” refers to a molecule or material that can produce a detectable (such as visually, electronically or otherwise) signal that indicates the presence and/or concentration of the label in a sample. When conjugated to a specific binding molecule, the detectable label can be used to locate and/or quantify the target to which the specific binding molecule is directed. Thereby, the presence and/or concentration of the target in a sample can be detected by detecting the signal produced by the detectable label. A detectable label can be detected directly or indirectly, and several different detectable labels conjugated to different specific-binding molecules can be used in combination to detect one or more targets. For example, a first detectable label conjugated to an antibody specific to a target can be detected indirectly through the use of a second detectable label that is conjugated to a molecule that specifically binds the first detectable label. Multiple detectable labels that can be separately detected can be conjugated to different specific binding molecules that specifically bind different targets to provide a multiplexed assay that can provide simultaneous detection of the multiple targets in a sample. A detectable signal can be generated by any mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected through antibody-hapten binding interactions using additional detectably labeled antibody conjugates, and paramagnetic and magnetic molecules or materials. Non-limiting examples of detectable labels include enzymes such as horseradish peroxidase, alkaline phosphatase, acid phosphatase, glucose oxidase, β-galactosidase or β-glucuronidase; fluorophores such as fluoresceins, luminophores, coumarins, BODIPY dyes, resorufins, and rhodamines (many additional examples of fluorescent molecules can be found in The Handbook—A Guide to Fluorescent Probes and Labeling Technologies, Molecular Probes, Eugene, Oreg.); nanoparticles such as quantum dots (obtained, for example, from QuantumDot Corp, Invitrogen Nanocrystal Technologies, Hayward, Calif; see also, U.S. Pat. Nos. 6,815,064, 6,682,596 and 6,649,138, each of which patents is incorporated by reference herein); metal chelates such as DOTA and DPTA chelates of radioactive or paramagnetic metal ions like Gd³⁺; and liposomes, for example, liposomes containing trapped fluorescent molecules. Where the detectable label includes an enzyme, a detectable substrate such as a chromogen, a fluorogenic compound, or a luminogenic compound can be used in combination with the enzyme to generate a detectable signal (A wide variety of such compounds are commercially available, for example, from Invitrogen Corporation, Eugene Oreg.). Non-limiting examples of chromogenic compounds include diaminobenzidine (DAB), 4-nitrophenylphospate (pNPP), fast red, bromochloroindolyl phosphate (BCIP), nitro blue tetrazolium (NBT), BCIP/NBT, fast red, AP Orange, AP blue, tetramethylbenzidine (TMB), 2,2′-azino-di-[3-ethylbenzothiazoline sulphonate] (ABTS), o-dianisidine, 4-chloronaphthol (4-CN), nitrophenyl-β-D-galactopyranoside (ONPG), o-phenylenediamine (OPD), 5-bromo-4-chloro-3-indolyl-β-galactopyranoside (X-Gal), methylumbelliferyl-β-D-galactopyranoside (MU-Gal), p-nitrophenyl-α-D-galactopyranoside (PNP), 5-bromo-4-chloro-3-indolyl-β-D-glucuronide (X-Gluc), 3-amino-9-ethyl carbazol (AEC), fuchsin, iodonitrotetrazolium (INT), tetrazolium blue and tetrazolium violet. Alternatively, an enzyme can be used in a metallographic detection scheme. Metallographic detection methods include using an enzyme such as alkaline phosphatase in combination with a water-soluble metal ion and a redox-inactive substrate of the enzyme. The substrate is converted to a redox-active agent by the enzyme, and the redox-active agent reduces the metal ion, causing it to form a detectable precipitate. (See, for example, co-pending U.S. patent application Ser. No. 11/015,646, filed Dec. 20, 2004, PCT Publication No. 2005/003777 and U.S. Patent Application Publication No. 2004/0265922; each of which is incorporated by reference herein). Metallographic detection methods include using an oxido-reductase enzyme (such as horseradish peroxidase) along with a water soluble metal ion, an oxidizing agent and a reducing agent, again to form a detectable precipitate. (See, for example, U.S. Pat. No. 6,670,113, which is incorporated by reference herein). Haptens are small molecules that are specifically bound by antibodies, although by themselves they will not elicit an immune response in an animal and must first be attached to a larger carrier molecule such as a protein to generate an immune response. Examples of haptens include di-nitrophenyl, biotin, digoxigenin, and fluorescein. Additional examples of oxazole, pyrazole, thiazole, nitroaryl, benzofuran, triperpene, urea, thiourea, rotenoid, coumarin and cyclolignan haptens are disclosed in U.S. Provisional Patent Application No. 60/856,133, filed Nov. 1, 2006, which is incorporated by reference herein. In some embodiments, the detectable label comprises a non-endogenous hapten (e.g. not biotin), such as, for example, the haptens disclosed in U.S. Pat. Nos. 7,695,929, 8,618,265 and 8,846,320 (incorporated herein by reference), including for example pyrazoles, nitrophenyl compounds, benzofurazans, triterpenes, ureas and thioureas, rotenone and rotenone derivatives, oxazoles and thiazoles, coumarin and coumarin derivatives, and cyclolignans.

The antigen binding domains or antibodies of the present disclosure may be multimerized to increase the affinity for an antigen. The antibody to be multimerized may be one type of antibody or a plurality of antibodies which recognize a plurality of epitopes of the same antigen. As a method of multimerization of the antibody, binding of the IgG CH3 domain to two scFv molecules, binding to streptavidin, introduction of a helix-turn-helix motif and the like can be exemplified.

The antigen binding domain or antibody compositions of the present disclosure may be in the form of a conjugate formed between polypeptide comprising the antigen binding domain or antibody and another agent (immunoconjugate). In one aspect, the polypeptides of the present disclosure are conjugated to radioactive material. In another aspect, the polypeptides of the present disclosure can be bound to various types of molecules such as polyethylene glycol (PEG).

The antigen binding domains or antibodies specific to HLA-A*11 can be conjugated to a therapeutic agent or effector molecule including, but are not limited to, molecules in which there is a covalent linkage of a therapeutic agent to an antibody. A therapeutic agent is an agent with a particular biological activity directed against a particular target molecule or a cell bearing a target molecule. One of skill in the art will appreciate that therapeutic agents can include various drugs such as vinblastine, daunomycin and the like, cytotoxins such as native or modified Pseudomonas exotoxin or Diphtheria toxin, encapsulating agents (such as liposomes) which themselves contain pharmacological compositions, radioactive agents such as ¹²⁵I, ³²P, ¹⁴C, ³H and ³⁵S and other labels, target moieties and ligands.

The choice of a particular therapeutic agent depends on the particular target molecule or cell, and the desired biological effect. Thus, for example, the therapeutic agent can be a cytotoxin that is used to bring about the death of a particular target cell (such as a tumor cell). Conversely, where it is desired to invoke a non-lethal biological response, the therapeutic agent can be conjugated to a non-lethal pharmacological agent or a liposome containing a non-lethal pharmacological agent.

With the therapeutic agents and antigen binding domains or antibodies described herein, one of skill can readily construct a variety of clones containing functionally equivalent nucleic acids, such as nucleic acids which differ in sequence but which encode the same effector moiety or antibody sequence. Thus, the present disclosure provides nucleic acids encoding antigen binding domains, antibodies and conjugates and fusion proteins thereof.

Effector molecules can be linked to a polypeptide comprising an HLA-A*11 antigen binding domain or antibody using any number of means known to those of skill in the art. Both covalent and noncovalent attachment means may be used. The procedure for attaching an effector molecule to an HLA-A*11 antigen binding domain or antibody varies according to the chemical structure of the effector. Polypeptides typically contain a variety of functional groups; such as carboxylic acid (COOH), free amine (—NH₂) or sulfhydryl (—SH) groups, which are available for reaction with a suitable functional group on an antibody to result in the binding of the effector molecule. Alternatively, the antibody is derivatized to expose or attach additional reactive functional groups. The derivatization may involve attachment of any of a number of known linker molecules. The linker can be any molecule used to join the antibody to the effector molecule. The linker is capable of forming covalent bonds to both the antibody and to the effector molecule. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (such as through a disulfide linkage to cysteine) or to the alpha carbon amino and carboxyl groups of the terminal amino acids.

In some circumstances, it is desirable to free the effector molecule from the antigen binding domain or antibody when the immunoconjugate has reached its target site. Therefore, in these circumstances, immunoconjugates will comprise linkages that are cleavable in the vicinity of the target site. Cleavage of the linker to release the effector molecule from the antigen binding domain or antibody may be prompted by enzymatic activity or conditions to which the immunoconjugate is subjected either inside the target cell or in the vicinity of the target site.

In view of the large number of methods that have been reported for attaching a variety of radiodiagnostic compounds, radiotherapeutic compounds, label (such as enzymes or fluorescent molecules) drugs, toxins, and other agents to proteins, one skilled in the art will be able to determine a suitable method for attaching a given agent to an antibody or antigen binding domain.

The antibodies or antigen binding domains disclosed herein can be derivatized or linked to another molecule (such as another peptide or protein). In some cases, the antibody, or an antibody fragment or derivative (such as a VH domain) is fused to a heterologous protein, for example an Fc protein. In some embodiments, the antibody or antibody fragment is fused to a part of a chimeric antigen receptor (CAR) protein.

In general, the antibodies or antigen binding domain is derivatized such that the binding to the target antigen is not affected adversely by the derivatization or labeling. For example, an antibody can be functionally linked (by chemical coupling, genetic fusion, noncovalent association or otherwise) to one or more other molecular entities, such as another antibody (for example, a bispecific antibody or a diabody), a detection agent, a pharmaceutical agent, and/or a protein or peptide that can mediate association of the antibody or antibody portion with another molecule (such as a streptavidin core region or a polyhistidine tag).

One type of derivatized antibody is produced by cross-linking two or more antibodies (of the same type or of different types, such as to create bispecific antibodies). Suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). Such linkers are commercially available.

An antibody or antigen binding domain that binds (for example specifically binds) an MHC I complex comprising HLA-A*11α chain can be labeled with a detectable moiety. Non-limiting examples of detection agents include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, Green fluorescent protein (GFP), Yellow fluorescent protein (YFP). An antibody can also be labeled with enzymes that are useful for detection, such as horseradish peroxidase, β-galactosidase, luciferase, alkaline phosphatase, glucose oxidase and the like. When an antibody is labeled with a detectable enzyme, it can be detected by adding additional reagents that the enzyme uses to produce a reaction product that can be discerned. For example, when the agent horseradish peroxidase is present the addition of hydrogen peroxide and diaminobenzidine leads to a colored reaction product, which is visually detectable. An antibody may also be labeled with biotin, and detected through indirect measurement of avidin or streptavidin binding. It should be noted that the avidin itself can be labeled with an enzyme or a fluorescent label.

An antibody or antigen binding domain may be labeled with a magnetic agent, such as gadolinium. Antibodies can also be labeled with lanthanides (such as europium and dysprosium), and manganese. Paramagnetic particles such as superparamagnetic iron oxide are also of use as labels. An antibody may also be labeled with a predetermined polypeptide epitopes recognized by a secondary reporter (such as leucine zipper pair sequences, binding sites for secondary antibodies, metal binding domains, epitope tags). In some embodiments, labels are attached by spacer arms of various lengths to reduce potential steric hindrance.

An antibody or antigen binding domain can also be labeled with a radiolabeled amino acid. The radiolabel may be used for both diagnostic and therapeutic purposes. For instance, the radiolabel may be used to detect MAGE-A3 by x-ray, emission spectra, or other diagnostic techniques. Examples of labels for polypeptides include, but are not limited to, the following radioisotopes or radionucleotides: ³H, ¹⁴C, ¹⁵N, ³⁵, ⁹⁰Y ⁹⁹Tc, ¹¹¹In, ¹²⁵I, ¹³¹I.

An antibody or antigen binding domain can also be derivatized with a chemical group such as polyethylene glycol (PEG), a methyl or ethyl group, or a carbohydrate group. These groups may be useful to improve the biological characteristics of the antibody, such as to increase serum half-life or to increase tissue binding.

Toxins can be employed with the antibodies or antigen binding domains described herein to produce immunotoxins. Exemplary toxins include ricin, abrin, diphtheria toxin and subunits thereof, as well as botulinum toxins A through F. These toxins are readily available from commercial sources (for example, Sigma Chemical Company, St. Louis, Mo.). Contemplated toxins also include variants of the toxins described herein (see, for example, see, U.S. Pat. Nos. 5,079,163 and 4,689,401). In some embodiments, the toxin is Pseudomonas exotoxin (PE) (U.S. Pat. No. 5,602,095). As used herein “Pseudomonas exotoxin” refers to a full-length native (naturally occurring) PE or a PE that has been modified. Such modifications can include, but are not limited to, elimination of domain Ia, various amino acid deletions in domains Ib, II and III, single amino acid substitutions and the addition of one or more sequences at the carboxyl terminus (for example, see Siegall et al., J Biol. Chem. 264:14256-14261, 1989).

PE employed with the antibodies or antigen binding domains described herein can include the native sequence, cytotoxic fragments of the native sequence, and conservatively modified variants of native PE and its cytotoxic fragments. Cytotoxic fragments of PE include those which are cytotoxic with or without subsequent proteolytic or other processing in the target cell. Cytotoxic fragments of PE include PE40, PE38, and PE35. For additional description of PE and variants thereof, see for example, U.S. Pat. Nos. 4,892,827; 5,512,658; 5,602,095; 5,608,039; 5,821,238; and 5,854,044; PCT Publication No. WO 99/51643; Pai et al., Proc. Natl. Acad. Sci. USA 88:3358-3362, 1991; Kondo et al., J. Biol. Chem. 263:9470-9475, 1988; Pastan et al., Biochim. Biophys. Acta 1333:C1-C6, 1997.

The antibodies or antigen binding domains described herein can also be used to target any number of different diagnostic or therapeutic compounds to cells expressing HLA-A*11. For example, an antibody of the present disclosure can be attached directly or via a linker to a drug that is to be delivered directly to cells expressing cell-surface HLA-A*11. This can be done for therapeutic, diagnostic or research purposes. Therapeutic agents include such compounds as nucleic acids, proteins, peptides, amino acids or derivatives, glycoproteins, radioisotopes, lipids, carbohydrates, or recombinant viruses. Nucleic acid therapeutic and diagnostic moieties include antisense nucleic acids, derivatized oligonucleotides for covalent cross-linking with single or duplex DNA, and triplex forming oligonucleotides.

Alternatively, the molecule linked to an anti-HLA-A*11 antibody can be an encapsulation system, such as a liposome or micelle that contains a therapeutic composition such as a drug, a nucleic acid (for example, an antisense nucleic acid), or another therapeutic moiety that is preferably shielded from direct exposure to the circulatory system. Means of preparing liposomes attached to antibodies are well known to those of skill in the art (see, for example, U.S. Pat. No. 4,957,735; Connor et al., Pharm. Ther. 28:341-365, 1985).

Antibodies described herein can also be covalently or non-covalently linked to a detectable label. Detectable labels suitable for such use include any composition detectable by spectroscopic, photochemical, biochemical, immunochemical, electrical, optical or chemical means. Useful labels include magnetic beads, fluorescent dyes (for example, fluorescein isothiocyanate, Texas red, rhodamine, green fluorescent protein, and the like), radiolabels (for example, ³H, ¹²⁵I, ³⁵S, ¹⁴C, or ³²P), enzymes (such as horseradish peroxidase, alkaline phosphatase and others commonly used in an ELISA), and colorimetric labels such as colloidal gold or colored glass or plastic (such as polystyrene, polypropylene, latex, and the like) beads.

Means of detecting such labels are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

### Receptors

The disclosure provides receptors comprising the polypeptides comprising the HLA-A*11 antigen binding domains described herein. Both chimeric antigen receptors (CARs) and T cell receptors (TCRs) are envisaged as within the scope of the instant disclosure. The CARs or TCRs, depending on architecture and choice of domain, can be either activator receptors or inhibitory receptors.

### Antigen Binding Domains

CARs and TCRs of the present disclosure comprise an HLA-A*11 antigen binding domain as described herein.

For example, the antigen binding domain of the receptor comprises HC CDRs selected from the group consisting of SEQ ID NOS: 1-19. In some embodiments, the antigen binding domain of the CAR or TCR comprises a HC CDR1 sequence selected from the group consisting of SEQ ID NOS: 1-5, a HC CDR2 sequence selected from the group consisting of SEQ ID NOS: 6-10, and a HC CDR3 sequence selected from the group consisting of SEQ ID NOS: 11-19. In some embodiments, the antigen binding domain of the CAR or TCR comprises a LC CDR1 comprising a sequence of SEQ ID NO: 20, a LC CDR2 comprising a sequence of SEQ ID NO: 21, and a LC CDR3 comprising a sequence of SEQ ID NO: 22. In some embodiments, the antigen binding domain of the CAR or TCR comprises: (a) a CDR1 sequence of SEQ ID NO: 1, a CDR2 sequence of SEQ ID NO: 6, and a CDR3 sequence of SEQ ID NO: 14; (b) a CDR1 sequence of SEQ ID NO: 2, a CDR2 sequence of SEQ ID NO: 7, and a CDR3 sequence of SEQ ID NO: 15; (c) a CDR1 sequence of SEQ ID NO: 1, a CDR2 sequence of SEQ ID NO: 6, and a CDR3 sequence of SEQ ID NO: 13; (d) a CDR1 sequence of SEQ ID NO: 5, a CDR2 sequence of SEQ ID NO: 10, and a CDR3 sequence of SEQ ID NO: 19; E a CDR1 sequence of SEQ ID NO: 1, a CDR2 sequence of SEQ ID NO: 6 and a CDR3 sequence of SEQ ID NO: 12; (f) a CDR1 sequence of SEQ ID NO: 4, a CDR2 sequence of SEQ ID NO: 9, and a CDR3 sequence of SEQ ID NO: 12; (g) a CDR1 sequence of SEQ ID NO: 4, a CDR2 sequence of SEQ ID NO: 9, and a CDR3 sequence of SEQ ID NO: 18; (h) a CDR1 sequence of SEQ ID NO: 3, a CDR2 sequence of SEQ ID NO: 8, and a CDR3 sequence of SEQ ID NO: 17; (i) a CDR1 sequence of SEQ ID NO: 2, a CDR2 sequence of SEQ ID NO: 7, and a CDR3 sequence of SEQ ID NO: 16; or (j) a CDR1 sequence of SEQ ID NO: 1, a CDR2 sequence of SEQ ID NO: 6, and a CDR3 sequence of SEQ ID NO: 11. In some embodiments, the HLA-A*11 antigen binding domain further comprises a light chain. In some embodiments, the light chain comprises CDRs of SEQ ID NOS: 20-22.

In some embodiments, the antigen binding domain of the receptor comprises a HC CDR1 comprising TSGVGVG (SEQ ID NO: 2), a HC CDR2 comprising LIYWNDDKRYSPSLKS (SEQ ID NO: 7), a HC CDR3 comprising KTTSFYFDY (SEQ ID NO: 15), a LC CDR1 comprising RASQSISSYLN (SEQ ID NO: 20), a LC CDR2 comprising AASSLQS (SEQ ID NO: 21), and a LC CDR3 comprising QQSYSTPLT (SEQ ID NO: 22).

In some embodiments, the antigen binding domain of the receptor comprises a HC CDR1 comprising SYWMH (SEQ ID NO: 5), a HC CDR2 comprising RINSDGSSTSYADSVKG (SEQ ID NO: 10), a HC CDR comprising GVLLYNWFDP (SEQ ID NO: 19), a LC CDR1 comprising RASQSISSYLN (SEQ ID NO: 20), a LC CDR2 comprising AASSLQS (SEQ ID NO: 21), and a LC CDR3 comprising QQSYSTPLT (SEQ ID NO: 22).

Antigen binding domains of the receptors of the disclosure may include, but are not limited to, fragment antigen-binding (Fab) fragments, single chain Fab (scFab) fragments, F(ab′)2 fragments, Fab′ fragments, Fv fragments, recombinant IgG (rIgG) fragments, single chain antibody fragments, single chain variable fragments (scFv), single domain antibodies (e.g., sdAb, sdFv, nanobody) fragments, diabodies, and multi-specific antibodies formed from antibody fragments. In particular embodiments, the antigen binding domains are single-chain antibody fragments comprising a variable heavy chain region and/or a variable light chain region, such as scFvs.

In some embodiments, the antigen-binding domain may comprise a scFv having a VH-linker-VL orientation. In some embodiments, the antigen-binding domain may comprise a scFv having a VL-linker-VH orientation.

In some embodiments, the antigen binding domain of the receptor is a scFv. In some embodiments, the scFv comprises a sequence of SEQ ID NOS: 23-31, or a sequence having at least 90%, at least 95%, at least 97%, at least 98% or at least 99% thereto. In some embodiments, the scFv comprises a sequence of SEQ ID NOS: 23-31.

In some embodiments, the antigen-binding domain further comprises a leader sequence or signal peptide. In embodiments where the antigen-binding domain comprises an scFv, the signal peptide may be positioned at the amino terminus of the scFv. In some embodiments, when the heavy chain variable region is N-terminal, the signal peptide may be positioned at the amino terminus of the heavy chain variable region. In some embodiments, when the light chain variable region is N-terminal, the signal peptide may be positioned at the amino terminus of the light chain variable region. The signal peptide may comprise any suitable signal peptide. In some embodiments, the signal peptide comprises the sequence of MDMRVPAQLLGLLLLWLRGARC (SEQ ID NO: 63).

### Chimeric Antigen Receptors (CARs)

The disclosure provides chimeric antigen receptors (CARs) specific to HLA-A*11. CARs of the disclosure comprise an extracellular antigen binding domain specific to HLA-A*11, as described supra, a transmembrane domain, and one or more intracellular domains. CARs specific to HLA-A*11 can be either activators, or inhibitors, of immune cell function.

In some embodiments, the CAR comprises a linker, spacer, or hinge sequence between the extracellular antigen binding domain and the transmembrane domain. One of ordinary skill in the art will appreciate that a hinge sequence is a short sequence of amino acids that, in at least some instances, facilitates flexibility (see, e.g., Woof et al., Nat. Rev. Immunol., 4(2): 89-99 (2004)). The hinge sequence can be any suitable sequence derived or obtained from any suitable molecule. In some embodiments, the length of the hinge sequence may be optimized based on the distance between the CAR and the HLA-A*11 epitope. The hinge may be derived from or include at least a portion of an immunoglobulin Fc region, for example, an IgG1 Fc region, an IgG2 Fc region, an IgG3 Fc region, an IgG4 Fc region, an IgE Fc region, an IgM Fc region, or an IgA Fc region. In certain embodiments, the spacer domain includes at least a portion of an IgG1, an IgG2, an IgG3, an IgG4, an IgE, an IgM, or an IgA immunoglobulin Fc region that falls within its CH2 and CH3 domains. In some embodiments, the spacer domain may also include at least a portion of a corresponding immunoglobulin hinge region. In some embodiments, the hinge is derived from or includes at least a portion of a modified immunoglobulin Fc region, for example, a modified IgG1 Fc region, a modified IgG2 Fc region, a modified IgG3 Fc region, a modified IgG4 Fc region, a modified IgE Fc region, a modified IgM Fc region, or a modified IgA Fc region. The modified immunoglobulin Fc region may have one or more mutations (e.g., point mutations, insertions, deletions, duplications) resulting in one or more amino acid substitutions, modifications, or deletions that cause impaired binding of the spacer domain to an Fc receptor (FcR). In some aspects, the modified immunoglobulin Fc region may be designed with one or more mutations which result in one or more amino acid substitutions, modifications, or deletions that cause impaired binding of the spacer domain to one or more FcR including, but not limited to, FcγRI, FcγR2A, FcγR2B1, FcγR2B2, FcγR3A, FcγR3B, FcεRI, FcεR2, FcαRI, Fcα/R, or FcRn.

In some embodiments, a portion of the immunoglobulin constant region serves as a hinge region between the antigen binding domain, e.g., scFv, and transmembrane domain. The spacer can be of a length that provides for increased responsiveness of the cell following antigen binding, as compared to in the absence of the spacer. Exemplary hinges include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a hinge has about 12 amino acids or less, about 119 amino acids or less, or about 229 amino acids or less. In some embodiments, the hinge is at or about 12 amino acids in length. Exemplary hinges include a CD28 hinge, IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153, international patent application publication number WO2014031687, U.S. Pat. No. 8,822,647 or published app. No. US2014/0271635.

In some embodiments, the CAR comprises a hinge domain isolated or derived from CD4, CD8α, IgG1, IgG2, or IgG4. In some embodiments, the hinge domain is isolated or derived from the human CD8α molecule or a CD28 molecule. In some embodiments, the hinge sequence is isolated or derived from CD8α.

Optionally, a short oligo- or polypeptide linker may form the linkage between the transmembrane domain and the intracellular signaling domain(s) of the CAR. A glycine-serine doublet may provide a suitable linker. Alternatively, or in addition, poly-glycine and poly-serine sequences may provide suitable linkers. In some embodiments, the polypeptide linker is between 2 and 10 amino acids in length.

With respect to the transmembrane domain, the CAR can be designed to comprise a transmembrane domain that is fused to the antigen-binding domain of the CAR. In some embodiments, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Typically, the transmembrane domain denotes a single transmembrane alpha helix of a transmembrane protein, also known as an integral protein. Transmembrane regions of particular use in this invention may be derived from (i.e. comprise at least the transmembrane region(s) of) CD28, CD3 epsilon, CD4, CD5, CD8, CD9, CD16, CD22, CD33, CD37, CD45, CD64, CD80, CD86, CD134, CD137, CD154, TCR alpha, TCR beta, or CD3 zeta and/or transmembrane regions containing functional variants thereof such as those retaining a substantial portion of the structural, e.g., transmembrane, properties thereof.

In some embodiments, the transmembrane domain comprises a comprises a transmembrane domain isolated or derived from CD8a molecule (CD8α), CD4 molecule (CD4), CD28 molecule (CD28), TNF receptor superfamily member 9 (CD137, or 4-1BB), CD80 molecule (CD80), CD86 molecule (CD86), cytotoxic T-lymphocyte associated protein 4 (CD152), programmed cell death 1 (PD-1), CD247 molecule (CD3ζ), or Fc fragment of IgE receptor Ig (FcRγ).

Alternatively, the transmembrane domain may be synthetic, in which case it will comprise predominantly hydrophobic residues such as leucine and valine. In some embodiments, a triplet of phenylalanine, tryptophan and valine can be used at one or both ends of a synthetic transmembrane domain. A transmembrane domain of the invention can be thermodynamically stable in a membrane. It may be a single alpha helix, a transmembrane beta barrel, a beta-helix of gramicidin A, or any other structure. In some embodiments, transmembrane helices are about 20 amino acids in length.

In some embodiments, the transmembrane domain in the CAR of the invention is the CD28 transmembrane domain.

In those embodiments wherein the CAR is an activator receptor, the intracellular signaling domain or otherwise the cytoplasmic domain of the CAR of the invention triggers or elicits activation of at least one of the normal effector functions of the immune cell in which the CAR has been placed. For example, the intracellular domain can be isolated or derived from an immune effector cell protein. The term “effector function” refers to a specialized function of a cell. Effector function of a T cell, for example, may be cytolytic activity or helper activity including the secretion of cytokines. Thus, the term “intracellular signaling domain” refers to the portion of a protein which transduces the effector function signal and directs the cell to perform a specialized function. While the entire intracellular signaling domain may be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Preferred examples of intracellular signaling domains for use in the activator CARs of the disclosure include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

Signals generated through one intracellular signaling domain alone may be insufficient for full activation of an immune cell, and a secondary or co-stimulatory signal may also be required. For example, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences). Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM-containing primary cytoplasmic signaling sequences that are useful as intracellular signaling domains according to the present disclosure include those derived from an intracellular signaling domain of a lymphocyte receptor chain, a TCR/CD3 complex protein, an Fc receptor subunit, an IL-2 receptor subunit, CD3ζ, FcRγ, FcRβ, CD3γ, CD3δ, CD3ε, CD5, CD22, CD79a, CD79b, CD66d, CD278(ICOS), FcεRI, DAP10, and DAP12. In some embodiments, the intracellular signaling domain in the CAR of the invention comprises a cytoplasmic signaling sequence derived from CD3. In some embodiments, the intracellular domain comprises an intracellular domain isolated or derived from CD3ζ.

In some embodiments of the activator CARs of the disclosure, the CAR comprises a costimulatory domain. The costimulatory domain refers to a portion of the CAR comprising the intracellular domain of a costimulatory molecule. A costimulatory molecule is a cell surface molecule other than an antigen receptor or their ligands that is required for an efficient response of lymphocytes to an antigen.

Various co-stimulatory domains have been reported to confer differing properties. For example, the 4-1BB co-stimulatory domain showed enhanced persistence in in vivo xenograph models (Milone et al. Mol Ther 2009; 17:1453-1464; Song et al. Cancer Res 2011; 71:4617-4627). Additionally, these different co-stimulatory domains produce different cytokine profiles which, in turn, may produce effects on target cell-mediated cytotoxicity and the tumor microenvironment. DAP10 signaling in NK cells has been associated with an increase in Th1 and inhibition of Th2 type cytokine production in CD8+ T cells (Barber et al. Blood 2011; 117:6571-6581).

Non-limiting examples of co-stimulatory molecules include an MHC class I molecule, TNF receptor proteins, immunoglobulin-like proteins, cytokine receptors, integrins, signaling lymphocytic activation molecules (SLAM proteins), activating NK cell receptors, a Toll ligand receptor, B7-H3, BAFFR, BTLA, BLAME (SLAMF8), CD2, CD4, CD5, CD7, CD8alpha, CD8beta, CD11a, LFA-1 (CD11a/CD18), CD11b, CD11c, CD11d, CD18, CD19, CD19a, CD27, CD28, CD29, CD30, CD40, CD49a, CD49D, CD49f, CD69, CD84, CD96 (Tactile), CD100 (SEMA4D), CD103, CRTAM, OX40 (CD134), 4-1BB (CD137), SLAM (SLAMFI, CD150, IPO-3), CD160 (BY55), SELPLG (CD162), DNAM1 (CD226), Ly9 (CD229), SLAMF4 (CD244, 2B4), ICOS (CD278), CEACAMI, CDS, CRTAM, DAP10, GADS, GITR, HVEM (LIGHTR), IA4, ICAM-1, IL2R beta, IL2R gamma, IL7R alpha, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, a ligand that specifically binds with CD83, and the like. Thus, while the invention is exemplified primarily with regions of CD28, and/or 4-1BB as the co-stimulatory signaling elements, other costimulatory elements are within the scope of the invention. In some embodiments, the co-stimulatory domain comprises a co-stimulatory domain isolated or derived from CD27 molecule (CD27), CD28, CD137, TNF receptor superfamily member 4 (OX40), TNF receptor superfamily member 8 (CD30), CD40 molecule (CD40), CD40 ligand (CD40L), CD3ζ, integrin subunit beta 2 (LFA-1), inducible T cell costimulator (ICOS), CD2 molecule (CD2), CD7 molecule (CD7), TNF superfamily member 14 (LIGHT), killer cell lectin like receptor C2 (NKG2C), CD276 molecule (B7-H3), or hematopoietic cell signal transducer (DAP10).

The cytoplasmic signaling sequences within the intracellular signaling domain of the CAR of the invention may be linked to each other in a random or specified order. Optionally, a short oligo- or polypeptide linker, may form the linkage. In some embodiments, the linker comprises glycine-serine doublet. In some embodiments, the linker is between 2 and 10 amino acids in length.

In some embodiments, the intracellular domain comprises the intracellular signaling domain of CD3ζ and a costimulatory domain derived from CD28. In some embodiments, the intracellular domain comprises the intracellular signaling domain of CD3ζ and a costimulatory domain derived from 4-1BB. In some embodiments, the intracellular domain comprises the intracellular signaling domain of CD3-ζ and costimulatory domains derived from both CD28 and 4-1BB.

In some embodiments, the hinge, transmembrane, and intracellular domains of the CAR comprise a sequence at least 90%, at least 95%, at least 99%, or 100% identical to TTTPAPRPPTPAPTIASQPLSLRPEACRPAAGGAVHTRGLDFACDFWVLVVVGGVLACYSLL VTVAFIIFWVRSKRSRLLHSDYMNMTPRRPGPTRKHYQPYAPPRDFAAYRSKRGRKKLLYIF KQPFMRPVQTTQEEDGCSCRFPEEEEGGCELRVKFSRSADAPAYKQGQNQLYNELNLGRREE YDVLDKRRGRDPEMGGKPRRKNPQEGLYNELQKDKMAEAYSEIGMKGERRRGKGHDGLY QGLSTATKDTYDALHMQALPPR (SEQ ID NO: 64). In some embodiments, the hinge, transmembrane, and intracellular domains of the CAR comprise, or consist essentially of, a sequence of SEQ ID NO: 64.

In some embodiments, the CAR is an inhibitory receptor. In some embodiments, the CAR comprises an HLA-A*11 extracellular domain, a transmembrane domain, and an inhibitory intracellular domain. In some embodiments, the CAR further comprises a hinge.

In some embodiments, the transmembrane domain or a functional variant thereof. In some embodiments, the LILRB1 transmembrane domain or a functional variant thereof comprises a sequence isolated or derived from LILRB1, as described in Table 5, below. In some embodiments, the transmembrane domain is not a LILRB1 transmembrane domain. The transmembrane domain may be derived either from a natural or from a recombinant source. Where the source is natural, the domain may be derived from any membrane-bound or transmembrane protein. Exemplary transmembrane domains may include at least the transmembrane region(s) of e.g., the alpha, beta or zeta chain of the TCR, CD3 delta, CD3 epsilon or CD3 gamma, CD28, CD3 epsilon, CD45, CD4, CD5, CD8, CD9, CD16, CD22, CD33, CD37, CD64, CD80, CD86, CD134, CD137, CD154. In some embodiments, the transmembrane comprises a TCR alpha transmembrane domain. In some embodiments, the TCR alpha transmembrane domain comprises an amino acid sequence having at least 85% identity, at least 90% identity, at least 95% identity, at least 96% identity, at least 97% identity, at least 98% identity, at least 99% identity or is identical to a sequence of: VIGFRILLLKVAGFNLLMTLRLW (SEQ ID NO: 113). In some embodiments, the transmembrane comprises a TCR beta transmembrane domain. In some embodiments, the TCR beta transmembrane domain comprises an amino acid sequence having at least 85% identity, at least 90% identity, at least 95% identity, at least 96% identity, at least 97% identity, at least 98% identity, at least 99% identity or is identical to a sequence of: TILYEILLGKATLYAVLVSALVL (SEQ ID NO: 114). In some embodiments the transmembrane domain comprise a CD28 transmembrane domain. In some embodiments, the CD28 transmembrane domain comprises an amino acid sequence having at least 80% identity, at least 90% identity, at least 95% identity, at least 99% identity or is identical to a sequence of FWVLVVVGGVLACYSLLVTVAFIIFWV (SEQ ID NO: 115).

In some embodiments, the transmembrane domain can be attached to the extracellular region chimeric antigen receptor, e.g., the antigen-binding domain or ligand binding domain, via a hinge, e.g., a hinge from a human protein. For example, in some embodiments, the hinge can be a human immunoglobulin (Ig) hinge, e.g., an IgG4 hinge, a CD8a hinge or an LILRB1 hinge, as described in Table 5 below. In some embodiments, the hinge is isolated or derived from CD8α or CD28. In some embodiments, the CD8a hinge comprises an amino acid sequence having at least 80% identity, at least 90% identity, at least 95% identity, at least 99% identity or is identical to a sequence of TTTPAPRPPTPAPTIASQPLSLRPEACRPAAGGAVHTRGLDFACD (SEQ ID NO: 116). In some embodiments, the CD28 hinge comprises an amino acid sequence having at least 80% identity, at least 90% identity, at least 95% identity, at least 99% identity or is identical to a sequence of CTIEVMYPPPYLDNEKSNGTIIHVKGKHLCPSPLFPGPSKP (SEQ ID NO: 117).

In some embodiments, for example those embodiments where the CAR is an inhibitor receptor, the intracellular domain of the CAR comprises an inhibitory domain. Inhibitory domain are capable of providing an inhibitory signal, and can comprise one or more motifs capable of providing the inhibitory signal, for example immunoreceptor tyrosine-based inhibitory motifs (ITIMs) and/or Src Homology 2 (SH2) domains. The ITIM can be natural, i.e. part of a domain isolated or derived from a protein, or non-naturally occurring or synthetic. In some aspects, the first intracellular domain comprises a programmed cell death 1 (PD-1) intracellular domain, a cytotoxic T-lymphocyte associated protein 4 (CTLA-4) intracellular domain, a killer cell immunoglobulin like receptor three Ig domains and long cytoplasmic tail 2 (KIR3DL2) intracellular domain, a killer cell immunoglobulin like receptor three Ig domains and long cytoplasmic tail 3 (KIR3DL3) intracellular domain, a zeta chain of T cell receptor associated protein kinase 70 (ZAP70) domain comprising a Src Homology 2 (SH2) domain, a ZAP70 inactivated kinase domain, a leukocyte immunoglobulin like receptor B1 (LIR1) intracellular domain, an Fc gamma receptor IIB (FcgRIIB) intracellular domain, or a killer cell lectin like receptor KI (NKG2D) intracellular domain. In some embodiments, the inhibitory intracellular domain is isolated or derived from leukocyte immunoglobulin like receptor B1 (LILRB1).

In some embodiments, the CAR comprises an intracellular domain isolated or derived from LILRB1, and one or more additional domains (transmembrane, and/or hinge) isolated or derived from LILRB1. In some embodiments, the inhibitory CAR comprises a hinge, transmembrane and transmembrane domain isolated or derived from LILRB1. In some embodiments, the hinge, transmembrane, and intracellular domains comprise a sequence at least 90%, at least 95%, at least 99%, or 100% identical to YGSQSSKPYLLTHPSDPLELVVSGPSGGPSSPTTGPTSTSGPEDQPLTPTGSDPQSGLG RHLGVVIGILVAVILLLLLLLLLFLILRHRRQGKHWTSTQRKADFQHPAGAVGPEPTD RGLQWRSSPAADAQEENLYAAVKHTQPEDGVEMDTRSPHDEDPQAVTYAEVKHSR PRREMASPPSPLSGEFLDTKDRQAEEDRQMDTEAAASEAPQDVTYAQLHSLTLRREA TEPPPSQEGPSPAVPSIYATLAIH (SEQ ID NO: 65). In some embodiments, the hinge, transmembrane, and intracellular domains comprise, or consist essentially of a sequence of SEQ ID NO: 65.

The HLA-A*11 specific CARs of the present disclosure, nucleotide sequences encoding the same, vectors encoding the same, and cells comprising nucleotide sequences encoding said CARs may be further modified, engineered, optimized, or appended in order to provide or select for various features. These features may include, but are not limited to, efficacy, persistence, target specificity, reduced immunogenicity, multi-targeting, enhanced immune response, expansion, growth, reduced off-tumor effect, reduced subject toxicity, improved target cytotoxicity, improved tumor infiltration, detection, selection, targeting, and the like. For example, the cells may be engineered to express another receptor, a suicide mechanism, and may be modified to remove or modify expression of an endogenous receptor or molecule such as a TCR and/or MHC molecule.

In some embodiments, the vector or nucleic acid sequence encoding the HLA-A*11 specific CAR further encodes other genes. The vector or nucleic acid sequence may be constructed to allow for the co-expression of multiple genes using a multitude of techniques including co-transfection of two or more plasmids, the use of multiple or bidirectional promoters, or the creation of bicistronic or multicistronic vectors. The construction of multicistronic vectors may include the encoding of IRES elements.

In some embodiments, the HLA-A*11 specific CAR is a regulatable CAR (RCAR), where the CAR activity can be controlled is desirable to optimize the safety and efficacy of a CAR therapy. In some embodiments, a RCAR comprises a set of polypeptides, typically two in the simplest embodiments, in which the components of a standard CAR described herein, e.g., an antigen-binding domain and an intracellular signaling domain, are partitioned on separate polypeptides or members. In some embodiments, the set of polypeptides include a dimerization switch that, upon the presence of a dimerization molecule, can couple the polypeptides to one another, e.g., can couple an antigen-binding domain to an intracellular signaling domain. Additional description and exemplary configurations of such regulatable CARs are provided herein and in International Publication No. WO 2015/090229, hereby incorporated by reference in its entirety.

In an aspect, an RCAR comprises two polypeptides or members: I) an intracellular signaling member comprising an intracellular signaling domain, e.g., a primary intracellular signaling domain described herein, and a first switch domain; 2) an antigen-binding member comprising an antigen-binding domain, e.g., that specifically binds a tumor antigen described herein, as described herein and a second switch domain. Optionally, the RCAR comprises a transmembrane domain described herein. In an embodiment, a transmembrane domain can be disposed on the intracellular signaling member, on the antigen-binding member, or on both. Unless otherwise indicated, when members or elements of an RCAR are described herein, the order can be as provided, but other orders are included as well. In other words, in an embodiment, the order is as set out in the text, but in other embodiments, the order can be different. E.g., the order of elements on one side of a transmembrane region can be different from the example, e.g., the placement of a switch domain relative to an intracellular signaling domain can be different, e.g., reversed.

In some embodiments, the antigen-binding domain of the HLA-A*11 specific CAR is or is part of an immunoconjugate, in which the antigen-binding domain is conjugated to one or more heterologous molecule(s), such as, but not limited to, a cytotoxic agent, an imaging agent, a detectable moiety a multimerization domain or other heterologous molecule. Cytotoxic agents include, but are not limited to, radioactive isotopes (e.g., At211, 1131, 1125, Y90, Re186, Re188, Sm153, Bi212, P32, Pb212 and radioactive isotopes of Lu); chemotherapeutic agents (e.g., methotrexate, adriamycin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins. In some embodiments, the antigen-binding domain is conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

Included in the scope of the invention are functional portions of the HLA-A*11 specific CARs described herein. The term “functional portion” when used in reference to a CAR refers to any part or fragment of the CAR of the invention, which part or fragment retains the biological activity of the CAR of which it is a part (the parent CAR). Functional portions encompass, for example, those parts of a CAR that retain the ability to recognize target cells, or detect, treat, or prevent a disease, to a similar extent, the same extent, or to a higher extent, as the parent CAR. In reference to the parent CAR, the functional portion can comprise, for instance, about 10%, 25%, 30%, 50%, 68%, 80%, 90%, 95%, or more, of the parent CAR.

The functional portion can comprise additional amino acids at the amino or carboxyl terminus of the portion, or at both termini, which additional amino acids are not found in the amino acid sequence of the parent CAR. In some embodiments, the additional amino acids do not interfere with the biological function of the functional portion, e.g., recognize target cells, detect cancer, treat or prevent cancer, etc. In some embodiments, the additional amino acids enhance the biological activity, as compared to the biological activity of the parent CAR.

Included in the scope of the invention are functional variants of the CARs described herein. The term “functional variant” as used herein refers to a CAR, polypeptide, or protein having substantial or significant sequence identity or similarity to a parent CAR, which functional variant retains the biological activity of the CAR of which it is a variant. Functional variants encompass those variants of the CAR described herein (the parent CAR) that retain the ability to recognize target cells to a similar extent, the same extent, or to a higher extent, as the parent CAR. In reference to the parent CAR, the functional variant can, for instance, be at least about 30%, 50%, 75%, 80%, 90%, 95%, 98%, 99% or 100% identical in amino acid sequence to the parent CAR. As is well known in the art, functional variants of antibodies, and by extension CARs comprising antibody fragments or antigen binding domains derived from antibodies, can be generated by varying the amino acids in the so-called framework regions of the antibody. Those of skill in the art can, through sequence alignment or structural modeling, identify residues that may be varied without loss of function and may select appropriate amino acid substitutions. Substitutions may include substitutions in solvent-exposed resides, often identified as non-conserved, polar, or hydrophilic. Interior residues may be varied by conservative substitutions—e.g., of one hydrophobic residue for another, or of residues of similar size or similar backbone rotational freedom. Structural models of representative VH and VL families have been published. Therefore it is possible, without undue effort, to identify reasonable substitutions to make based on such published structures. These techniques may be used to humanize antibodies. Furthermore, using methods disclosed herein or known in the art, one may with routine experimentation confirm that variants retain their desired function—e.g., specific binding to a target. Functional variants of antibodies are not limited to substitution in the framework regions. For example, CDR sequences may be swapped between antibodies having a common target specificity and tested with routine experimentation. CDR sequences are not fixed either. Point mutations or insertion may be designed using techniques well known in the art, and testing of such variants is routine. Moreover, VH-VL pairings can be varied. The preferred pairing partners for each VH and VL type are well known. Thus, is it not necessary to test every possible VH and VL pair. Rather, those skilled in the art are capable of rationally selecting candidate pairings. For all these reasons, the scope of the present disclosure is not intended to limit the invention to only the exact amino acid sequences disclosed herein. In particular, polypeptides of the disclosure may have amino acid substitutions and insertion in the framework regions of the antigen binding domain so long as the function of the antigen binding domain is retained, which could be confirm with routine experimentation using isolated antigen binding domains or functional CARs expressed in cells.

A functional variant can, for example, comprise the amino acid sequence of the parent CAR with at least one conservative amino acid substitution. Alternatively or additionally, the functional variants can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

Amino acid substitutions of the inventive CARs are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gin, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

Also, amino acids may be added or removed from the sequence based on vector design.

The receptors can consist essentially of the specified amino acid sequence or sequences described herein, such that other components, e.g., other amino acids, do not materially change the biological activity of the functional variant.

The CARs of embodiments of the invention (including functional portions and functional variants) can be of any length, i.e., can comprise any number of amino acids, provided that the CARs (or functional portions or functional variants thereof) retain their biological activity, e.g., the ability to specifically bind to antigen, detect diseased cells in a mammal, or treat or prevent disease in a mammal, etc. For example, the CAR can be about 50 to about 5000 amino acids long, such as 50, 70, 75, 100, 125, 150, 175, 200, 300, 400, 500, 600, 700, 800, 900, 1000 or more amino acids in length.

The CARs of embodiments of the invention (including functional portions and functional variants of the invention) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, a-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N‘-benzyl-N’-methyl-lysine, N′,N′-dibenzyl-lysine, 6-hydroxylysine, ornithine, u-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and a-tert-butylglycine.

In some embodiments, CARs (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated, or undergo additional post-translational modifications.

Receptors (including functional portions and functional variants thereof) can be obtained by methods known in the art. The receptors may be made by any suitable method of making polypeptides or proteins. Suitable methods of de novo synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; Epitope Mapping, ed. Westwood et al., Oxford University Press, Oxford, United Kingdom, 2001; and U.S. Pat. No. 5,449,752. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, N Y, 1994. Further, some of the CARs of the invention (including functional portions and functional variants thereof) can be isolated and/or purified from a source, such as a plant, a bacterium, an insect, a mammal, e.g., a rat, a human, etc. Methods of isolation and purification are well-known in the art. Alternatively, the CARs described herein (including functional portions and functional variants thereof) can be commercially synthesized by companies. In this respect, the receptors can be synthetic, recombinant, isolated, and/or purified.

### T Cell Receptors (TCRs)

The disclosure provides TCRs comprising the HLA-A*11 antigen binding domains of the disclosure. TCRs can be activator receptors, or can be engineered to act as inhibitory receptors through the inclusion of inhibitory intracellular domains, as described herein.

HLA-A*11 antigen binding domains of the disclosure can be fused to any one or more of the TCRα, TCRβ, CD3ζ, CD3δ, CD3ε or CD3γ subunits of the TCR. For example, all or part of the endogenous extracellular antigen binding domain of TCRα and/or TCRβ can be replaced with an HLA-A*11 antigen binding domain fused to one or both of TCRα and/or TCRβ subunits. As a further example, TCRs of the disclosure can comprise an HLA-A*11 antigen binding domain that is an scFv comprising a sequence of SEQ ID NOS: 23-31, or a sequence having at least 90%, at least 95%, at least 97%, at least 98% or at least 99% thereto, fused to TCRα, TCRβ, CD3ζ, CD3δ, CD3ε or CD3γ, or a combination thereof. In some embodiments, the TCR comprises a scFv comprising a sequence of SEQ ID NOS: 23-31 fused to TCRα, TCRβ, CD3ζ, CD3δ, CD3ε or CD3γ, or a combination thereof.

In some embodiments, the TCR further comprises one or more additional intracellular domains that enhance and or alter the activity of the TCR. Additional intracellular domains can be fused to any one or more of TCRα, TCRβ, CD3ζ, CD3δ, CD3ε or CD3γ subunits of the TCR, in place of, or in addition to, the endogenous intracellular domain of the TCR subunit.

In some embodiments, the TCR comprises an intracellular domain that provides an activator signal to an immune cell expressing the TCR, thereby enhancing the activity of the TCR. TCRs comprising additional activator domains are described in WO 2021/030153, the contents of which are incorporated by reference in their entirety herein. Exemplary activator domains for use with the TCRs of the disclosure include, but are not limited to, domains isolated or derived from CD28 molecule (CD28), TNF receptor superfamily member 9 (4-1BB), TNF receptor superfamily member 18 (GITR), LCK proto-oncogene, Src family tyrosine kinase (Lck), CD4 molecule (CD4), CD8a molecule (CD8), FYN proto-oncogene, Src family tyrosine kinase (Fyn), zeta chain of T cell receptor associated protein kinase 70 (ZAP70), linker for activation of T cells (LAT), and lymphocyte cytosolic protein 2 (SLP76).

In some embodiments, the TCR comprises an intracellular domain that provides an inhibitory signal to an immune cell expressing the TCR, for example immunoreceptor tyrosine-based inhibitory motifs (ITIMs) and/or Src Homology 2 (SH2) domains. Exemplary intracellular domains capable of providing an inhibitory signal are described in PCT/US2020/059856 filed on Nov. 10, 2020, the contents of which are incorporated by reference in their entirety herein, and are described in further detail below.

In some embodiments, the inhibitory domain of the TCR is isolated or derived from LILRB1. LILRB1 domains are described in more detail, below. For example, an inhibitory TCR can comprise TCRα or TCRβ subunit comprising an HLA-A*11 antigen binding domain, a transmembrane domain from TCRα or TCRβ, respectively, and a LILRB1 intracellular domain. As a further example, an inhibitory TCR can comprise a subunit comprising a LILRB1 intracellular domain, and a LILRB1 transmembrane domain.

### Inhibitory Domains

The disclosure provides receptors, such as CARs and TCRs, comprising an HLA-A*11 antigen binding domain and an inhibitory domain.

In some embodiments, the inhibitory intracellular domain comprises one or more ITIMs. As used herein an “immunoreceptor tyrosine-based inhibitory motif” or “ITIM” refers to a conserved sequence of amino acids with a consensus sequence of S/I/V/LxYxxI/V/L (SEQ ID NO: 66) that is found in the cytoplasmic tails of many inhibitory receptors of the immune system. After ITIM-possessing inhibitory receptors interact with their ligand, the ITIM motif is phosphorylated, allowing the inhibitory receptor to recruit other enzymes, such as the phosphotyrosine phosphatases SHP-1 and SHP-2, or the inositol-phosphatase called SHIP.

In some embodiments, the inhibitory intracellular domain is selected from the intracellular domains of CTLA4, PD1, lymphocyte activating 3 (LAG3), hepatitis A virus cellular receptor 2 (HAVCR2, also referred to as TIM3), KIR2DL2, KIR3DL2, LILRB1 (LIR1), T cell immunoreceptor with Ig and ITIM domains (TIGIT), CEA cell adhesion molecule 1 (CEACAMI), colony stimulating factor 1 receptor (CSF1R), CD5, CD96 molecule (CD96), CD22 molecule (CD22) and leukocyteassociated immunoglobulin like receptor 1 (LAIR1) or functional fragments thereof. Further inhibitory domains include those described in International Application Pub. No. WO2016075612A1. In some embodiments, the domain capable of providing an inhibitory signal is selected from the intracellular domain of a leukocyte immunoglobulin-like receptors (LIR) or a functional fragment thereof. In some embodiments, the domain is selected from the intracellular domain of LILRB1, LILRB2, LILRB3, LILRB4, and LILRB5, or functional fragments thereof. In some embodiments, the domain is selected from the intracellular domain of PIR-B or a functional fragment thereof. In some embodiments, the domain is the intracellular domain of LILRB1. In some embodiments, the domain is a functional fragment of the intracellular domain of LILRB1. In some embodiments, the domain capable of providing an inhibitory signal is selected from the intracellular domain of an inhibitory killer cell immunoglobulin like receptor (KIR) or a functional fragment thereof. In some embodiments, the domain is selected from the intracellular domain of KIR2DL1, KIR2DL2, KIR2DL3, KIR2DL4, KIR2DL5A, KIR2DL5B, KIR3DL1, KIR3DL2, and KIR3DL3, or functional fragments thereof.

In some embodiments, the inhibitory domain is isolated or derived from LILRB1 (also referred to as LIR1, or LIR-1). In some embodiments, further domains of the receptor are also isolated or derived from LILRB1, such as the transmembrane domain and/or hinge. Exemplary LILRB1 sequences for use in the inhibitory receptors described herein are provided in Table 5 below. LILRB1 based inhibitory receptors are described in PCT/US2020/064607 filed on Dec. 11, 2020, the contents of which are incorporated by reference in their entirety herein.

TABLE 5
Polypeptide Sequences for Elements of Illustrative Chimeric Antigen Receptors

| Name | Sequence |
|---|---|
| LILRB1 | MTPILTVLICLGLSLGPRTHVQAGHLPKPTLWAEPGSVITQ |
| | GSPVTLRCQGGQETQEYRLYREKKTALWITRIPQELVKKG |
| | QFPIPSITWEHAGRYRCYYGSDTAGRSESSDPLELVVTGAYI |
| | KPTLSAQPSPVVNSGGNVILQCDSQVAFDGFSLCKEGEDEH |
| | PQCLNSQPHARGSSRAIFSVGPVSPSRRWWYRCYAYDSNS |
| | PYEWSLPSDLLELLVLGVSKKPSLSVQPGPIVAPEETLTLQC |
| | GSDAGYNRFVLYKDGERDFLQLAGAQPQAGLSQANFTLG |
| | PVSRSYGGQYRCYGAHNLSSEWSAPSDPLDILIAGQFYDRV |
| | SLSVQPGPTVASGENVTLLCQSQGWMQTFLLTKEGAADDP |
| | WRLRSTYQSQKYQAEFPMGPVTSAHAGTYRCYGSQSSKP |
| | YLLTHPSDPLELVVSGPSGGPSSPTTGPTSTSGPEDQPLTPT |
| | GSDPQSGLGRHLGVVIGILVAVILLLLLLLLLFLILRHRRQG |
| | KHWTSTQRKADFQHPAGAVGPEPTDRGLQWRSSPAADAQ |
| | EENLYAAVKHTQPEDGVEMDTRSPHDEDPQAVTYAEVKH |
| | SRPRREMASPPSPLSGEFLDTKDRQAEEDRQMDTEAAASE |
| | APQDVTYAQLHSLTLRREATEPPPSQEGPSPAVPSIYATLAI |
| | H (SEQ ID NO: 62) |
| LILRB1 hinge- | YGSQSSKPYLLTHPSDPLELVVSGPSGGPSSPTTGPTSTSG |
| transmembrane- | PEDQPLTPTGSDPQSGLGRHLGVVIGILVAVILLLLLLLLL |
| intracellular | FLILRHRRQGKHWTSTQRKADFQHPAGAVGPEPTDRGLQ |
| domain | WRSSPAADAQEENLYAAVKHTQPEDGVEMDTRSPHDEDP |
| | QAVTYAEVKHSRPRREMASPPSPLSGEFLDTKDRQAEEDR |
| | QMDTEAAASEAPQDVTYAQLHSLTLRREATEPPPSQEGPSP |
| | AVPSIYATLAIH |
| | (SEQ ID NO: 65) |
| LILRB1 hinge- | VVSGPSGGPSSPTTGPTSTSGPEDQPLTPTGSDPQSGLGRHL |
| transmembrane- | GVVIGILVAVILLLLLLLLLFLILRHRRQGKHWTSTQRKA |
| intracellular | DFQHPAGAVGPEPTDRGLQWRSSPAADAQEENLYAAVKH |
| domain (w/o | TQPEDGVEMDTRSPHDEDPQAVTYAEVKHSRPRREMASPP |
| YGSQSSKPYLL | SPLSGEFLDTKDRQAEEDRQMDTEAAASEAPQDVTYAQLH |
| THPSDPLEL, | SLTLRREATEPPPSQEGPSPAVPSIYATLAIH (SEQ ID NO: 67) |
| SEQ ID NO: 66) | |
| LILRB1 hinge | YGSQSSKPYLLTHPSDPLELVVSGPSGGPSSPTTGPTSTSG |
| domain | PEDQPLTPTGSDPQSGLGRHLG (SEQ ID NO: 68) |
| LILRB1 | VVIGILVAVILLLLLLLLLFLIL (SEQ ID NO: 69) |
| transmembrane | |
| domain | |
| LILRB1 | RHRRQGKHWTSTQRKADFQHPAGAVGPEPTDRGLQWRSS |
| intracellular | PAADAQEENLYAAVKHTQPEDGVEMDTRSPHDEDPQAVT |
| domain | YAEVKHSRPRREMASPPSPLSGEFLDTKDRQAEEDRQMDT |
| | EAAASEAPQDVTYAQLHSLTLRREATEPPPSQEGPSPAVPSI |
| | YATLAIH (SEQ ID NO: 70) |
| ITIM1 | NLYAAV (SEQ ID NO: 71) |
| ITIM2 | VTYAEV (SEQ ID NO: 72) |
| ITIM3 | VTYAQL (SEQ ID NO: 73) |
| ITIM4 | SIYATL (SEQ ID NO: 74) |
| ITIM1-2 | NLYAAVKHTQPEDGVEMDTRSPHDEDPQAVTYAEV (SEQ |
| | ID NO: 75) |
| ITIM2-3 | VTYAEVKHSRPRREMASPPSPLSGEFLDTKDRQAEEDRQM |
| | DTEAAASEAPQDVTYAQL (SEQ ID NO: 76) |
| ITIM3-4 | VTYAQLHSLTLRREATEPPPSQEGPSPAVPSIYATL (SEQ ID |
| | NO: 77) |
| ITIM1-3 | NLYAAVKHTQPEDGVEMDTRSPHDEDPQAVTYAEVKHSR |
| | PRREMASPPSPLSGEFLDTKDRQAEEDRQMDTEAAASEAP |
| | QDVTYAQL (SEQ ID NO: 78) |
| ITIM2-4 | VTYAEVKHSRPRREMASPPSPLSGEFLDTKDRQAEEDRQM |
| | DTEAAASEAPQDVTYAQLHSLTLRREATEPPPSQEGPSPAV |
| | PSIYATL (SEQ ID NO: 79) |
| ITIM1-4 | NLYAAVKHTQPEDGVEMDTRSPHDEDPQAVTYAEVKHSR |
| | PRREMASPPSPLSGEFLDTKDRQAEEDRQMDTEAAASEAP |
| | QDVTYAQLHSLTLRREATEPPPSQEGPSPAVPSIYATL |
| | (SEQ ID NO: 80) |
| D3D4 domain | YGSQSSKPYLLTHPSDPLEL (SEQ ID NO: 81) |
| Short hinge | VVSGPSGGPSSPTTGPTSTSGPEDQPLTPTGSDPQSGLGRHL |
| | G (SEQ ID NO: 82) |
| Hinge (iTIM | YGSQSSKPYLLTHPSDPLELVVSGPSGGPSSPTTGPTSTSGP |
| hinge) | EDQPLTPTGSDPQSGLGRHLGV (SEQ ID NO: 83) |
| Short hinge 2 | VVSGPSGGPSSPTTGPTSTSGPEDQPLTPTGSDPQSGLGRHL |
| | GV (SEQ ID NO: 84) |
| Long hinge 1 | AGSGGSGGSGGSPVPSTPPTPSPSTPPTPSPSGGSGNSSGSGG |
| | SPVPSTPPTPSPSTPPTPSPSASV (SEQ ID NO: 85) |
| Long hinge 2 | AGSGGSGGSGGSPVPSTPPTNSSSTPPTPSPSPVPSTPPTNSSS |
| | TPPTPSPSPVPSTPPTNSSSTPPTPSPSASV (SEQ ID NO: 86) |
| 2X short hinge | VVSGPSGGPSSPTTGPTSTSGPEDQPLTPTGSDPQSGLGRHV |
| | VSGPSGGPSSPTTGPTSTSGPEDQPLTPTGSDPQSGLGRHLG |
| | V (SEQ ID NO: 87) |
| Hinge (truncated) | TTGPTSTSGPEDQPLTPTGSDPQSGLGRHLGV (SEQ ID NO: |
| | 135) |
| Hinge- | YGSQSSKPYLLTHPSDPLELVVSGPSGGPSSPTTGPTSTSG |
| transmembrane | PEDQPLTPTGSDPQSGLGRHLGVVIGILVAVILLLLLLLLL |
| | FLIL (SEQ ID NO: 88) |
| Transmembrane- | VVIGILVAVILLLLLLLLLFLILRHRRQGKHWTSTQRKAD |
| intracellular | FQHPAGAVGPEPTDRGLQWRSSPAADAQEENLYAAVKHT |
| domain. | QPEDGVEMDTRSPHDEDPQAVTYAEVKHSRPRREMASPPS |
| | PLSGEFLDTKDRQAEEDRQMDTEAAASEAPQDVTYAQLHS |
| | LTLRREATEPPPSQEGPSPAVPSIYATLAIH (SEQ ID NO: 89) |

In some embodiments, the inhibitory domain comprises an intracellular domain isolated or derived from a CD200 receptor. The cell surface glycoprotein CD200 receptor 1 (Uniprot ref: Q8TD46) represents another example of an inhibitory intracellular domain of the present invention. This inhibitory receptor for the CD200/OX2 cell surface glycoprotein limits inflammation by inhibiting the expression of proinflammatory molecules including TNF-alpha, interferons, and inducible nitric oxide synthase (iNOS) in response to selected stimuli.

In some embodiments, the inhibitory domain is isolated or derived from a human protein. In some embodiments, the inhibitory domain is isolated or derived from a human TRAIL receptor, CD200 receptor 1, PD-1, CTLA-4, KIR3DL2, KIR3DL3, ZAP70, LIR1, FcgRIIB or NKG2D.

In some embodiments, the inhibitory domain is selected from the group consisting of a PD-1 intracellular domain, a LIR1 intracellular domain, and a KIR3DL2 intracellular domain.

In some embodiments, the inhibitory domain comprises a KIR3DL2 intracellular domain or a functional fragment thereof. In some embodiments, KIR3DL2 comprises an amino acid sequence having at least 85% identity, at least 90% identity, at least 95% identity, at least 96% identity, at least 97% identity, at least 98% identity, at least 99% identity or is identical to a sequence or subsequence of:

| (SEQ ID NO: 118) | |
|---|---|
| 1 | MSLTVVSMAC VGFFLLQGAW PLMGGQDKPF LSARPSTVVP RGGHVALQCH YRRGFNNFML |
| 61 | YKEDRSHVPI FHGRIFQESF IMGPVTPAHA GTYRCRGSRP HSLTGWSAPS NPLVIMVTGN |
| 121 | HRKPSLLAHP GPLLKSGETV ILQCWSDVMF EHFFLHREGI SEDPSRLVGQ IHDGVSKANF |
| 181 | SIGPLMPVLA GTYRCYGSVP HSPYQLSAPS DPLDIVITGL YEKPSLSAQP GPTVQAGENV |
| 241 | TLSCSSWSSY DIYHLSREGE AHERRLRAVP KVNRTFQADF PLGPATHGGT YRCFGSFHAL |
| 301 | PCVWSNSSDP LLVSVTGNPS SSWPSPTEPS SKSGICRHLH VLIGTSVVIF LFILLLFFLL |
| 361 | YRWCSNKKNA AVMDQEPAGD RTVNRQDSDE QDPQEVTYAQ LDHCVFIQRK ISRPSQRPKT |
| 421 | PLTDTSVYTE LPNAEPRSKV VSCPRAPQSG LEGVF. |

In some embodiments, the KIR3DL3 domain comprises an amino acid sequence having at least 85% identity, at least 90% identity, at least 95% identity, at least 96% identity, at least 97% identity, at least 98% identity, at least 99% identity or is identical to a fragment of SEQ ID NO: 118.

In some embodiments, the KIR3DL2 domain comprises an amino acid sequence having at least 85% identity, at least 90% identity, at least 95% identity, at least 96% identity, at least 97% identity, at least 98% identity, at least 99% identity or is identical to a sequence of NKKNAAVMDQEPAGDRTVNRQDSDEQDPQEVTYAQLDHCVFIQRKISRPSQRPKTPLTDTSV YTELPNAEPRSKVVSCPRAPQSGLEGVF (SEQ ID NO: 119). In some embodiments, the KIR3DL2 domain comprises or consists essentially of SEQ ID NO: 119.

In some embodiments, the inhibitory domain comprises a KIR3DL3 domain or a functional fragment thereof. In some embodiments, KIR3DL3 comprises an amino acid sequence having at least 85% identity, at least 90% identity, at least 95% identity, at least 96% identity, at least 97% identity, at least 98% identity, at least 99% identity or is identical to a sequence or subsequence of:

| (SEQ ID NO: 120) | |
|---|---|
| 1 | MSLMVVSMAC VGFFLLEGPW PHVGGQDKPF LSAWPSTVVS EGQHVTLQCR SRLGFNEFSL |
| 61 | SKEDGMPVPE LYNRIFRNSF LMGPVTPAHA GTYRCCSSHP HSPTGWSAPS NPVVIMVTGV |
| 121 | HRKPSLLAHP GPLVKSGETV ILQCWSDVRF ERFLLHREGI TEDPLRLVGQ LHDAGSQVNY |
| 181 | SMGPMTPALA GTYRCFGSVT HLPYELSAPS DPLDIVVVGL YGKPSLSAQP GPTVQAGENV |
| 241 | TLSCSSRSLF DIYHLSREAE AGELRLTAVL RVNGTFQANF PLGPVTHGGN YRCFGSFRAL |
| 301 | PHAWSDPSDP LPVSVTGNSR YLHALIGTSV VIIPFAILLF FLLHRWCANK KNAVVMDQEP |
| 361 | AGNRTVNRED SDEQDPQEVT YAQLNHCVFT QRKITRPSQR PKTPPTDTSV. |

In some embodiments, the KIR3DL3 domain comprises an amino acid sequence having at least 85% identity, at least 90% identity, at least 95% identity, at least 96% identity, at least 97% identity, at least 98% identity, at least 99% identity or is identical to a fragment of SEQ ID NO: 120.

In some embodiments, the inhibitory domain comprises ZAP70, a ZAP70 domain, or a functional fragment thereof. In some embodiments, the ZAP70 domain comprises one or more ZAP70 SH2 domains. The ZAP70 protein comprises two SH2 domains, referred to herein as the N and C terminal SH2 domains. In some embodiments, the ZAP70 N terminal SH2 domain comprises a sequence of FFYGSISRAEAEEHLKLAGMADGLFLLRQCLRSLGGYVLSLVHDVRFHHFPIERQLN GTYAIAGGKAHCGPAELCEFYSRDPDGLPCNLRKPC (SEQ ID NO: 121), or at least 85% identity, at least 90% identity, at least 95% identity, at least 96% identity, at least 97% identity, at least 98% identity, at least 99% identity thereto. In some embodiments, the ZAP70 C terminal SH2 domain comprises a sequence of WYHSSLTREEAERKLYSGAQTDGKFLLRPRKEQGTYALSLIYGKTVYHYLISQDKAG KYCIPEGTKFDTLWQLVEYLKLKADGLIYCLKEAC (SEQ ID NO: 122), or at least 85% identity, at least 90% identity, at least 95% identity, at least 96% identity, at least 97% identity, at least 98% identity, at least 99% identity thereto. In some embodiments, the ZAP70 domain comprises an amino acid sequence having at least 85% identity, at least 90% identity, at least 95% identity, at least 96% identity, at least 97% identity, at least 98% identity, at least 99% identity or is identical to a sequence or partial sequence of:

| (SEQ ID NO: 123) | |
|---|---|
| 1 | MPDPAAHLPF FYGSISRAEA EEHLKLAGMA DGLFLLRQCL RSLGGYVLSL VHDVRFHHFP |
| 61 | IERQLNGTYA IAGGKAHCGP AELCEFYSRD PDGLPCNLRK PCNRPSGLEP QPGVFDCLRD |
| 121 | AMVRDYVRQT WKLEGEALEQ AIISQAPQVE KLIATTAHER MPWYHSSLTR EEAERKLYSG |
| 181 | AQTDGKFLLR PRKEQGTYAL SLIYGKTVYH YLISQDKAGK YCIPEGTKFD TLWQLVEYLK |
| 241 | LKADGLIYCL KEACPNSSAS NASGAAAPTL PAHPSTLTHP QRRIDTLNSD GYTPEPARIT |
| 301 | SPDKPRPMPM DTSVYESPYS DPEELKDKKL FLKRDNLLIA DIELGCGNFG SVRQGVYRMR |
| 361 | KKQIDVAIKV LKQGTEKADT EEMMREAQIM HQLDNPYIVR LIGVCQAEAL MLVMEMAGGG |
| 421 | PLHKFLVGKR EEIPVSNVAE LLHQVSMGMK YLEEKNFVHR DLAARNVLLV NRHYAKISDF |
| 481 | GLSKALGADD SYYTARSAGK WPLKWYAPEC INFRKFSSRS DVWSYGVTMW EALSYGQKPY |
| 541 | KKMKGPEVMA FIEQGKRMEC PPECPPELYA LMSDCWIYKW EDRPDFLTVE QRMRACYYSL |
| 601 | ASKVEGPPGS TQKAEAACA. |

In some embodiments, the ZAP70 or a portion thereof is kinase inactive. In some embodiments, the kinase inactive ZAP70 comprises an amino acid substitution of Alanine for Lysine at position 369 of SEQ ID NO: 123.

In some embodiments, the inhibitory domain comprises a LILRB2 domain or a functional fragment thereof. Inclusion of the LILRB2 domain in the engineered TCR can inhibit T cell activation. In some embodiments, LILRB2 comprises an amino acid sequence having at least 85% identity, at least 90% identity, at least 95% identity, at least 96% identity, at least 97% identity, at least 98% identity, at least 99% identity or is identical to a sequence of:

(SEQ ID NO: 124)
LRHRRQGKHWTSTQRKADFQHPAGAVGPEPTDRGLQWRSSPAADAQEEN
LYAAVKDTQPEDGVEMDTRAAASEAPQDVTYAQLHSLTLRRKATEPPPS
QEREPPAEPSIYATLAIH.

In some embodiments, the LILRB2 domain comprises an amino acid sequence having at least 85% identity, at least 90% identity, at least 95% identity, at least 96% identity, at least 97% identity, at least 98% identity, at least 99% identity or is identical to a fragment of SEQ ID NO: 124.

In some embodiments, the inhibitory domain comprises a LILRB3 domain or a functional fragment thereof. Inclusion of the LILRB3 domain in the engineered TCR can inhibit T cell activation. In some embodiments, LILRB3 comprises an amino acid sequence having at least 85% identity, at least 90% identity, at least 95% identity, at least 96% identity, at least 97% identity, at least 98% identity, at least 99% identity or is identical to a sequence of

(SEQ ID NO: 125)
RRQRHSKHRTSDQRKTDFQRPAGAAETEPKDRGLLRRSSPAADVQEENL
YAAVKDTQSEDRVELDSQSPHDEDPQAVTYAPVKHSSPRREMASPPSSL
SGEFLDTKDRQVEEDRQMDTEAAASEASQDVTYAQLHSLTLRRKATEPP
PSQEGEPPAEPSIYATLAIH.

In some embodiments, the LILRB3 domain comprises an amino acid sequence having at least 85% identity, at least 90% identity, at least 95% identity, at least 96% identity, at least 97% identity, at least 98% identity, at least 99% identity or is identical to a fragment of SEQ ID NO: 125.

In some embodiments, the inhibitory domain comprises a LILRB4 domain or a functional fragment thereof. Inclusion of the LILRB4 domain in the engineered TCR can inhibit T cell activation. In some embodiments, LILRB4 comprises an amino acid sequence having at least 85% identity, at least 90% identity, at least 95% identity, at least 96% identity, at least 97% identity, at least 98% identity, at least 99% identity or is identical to a sequence of:

(SEQ ID NO: 126)
QHWRQGKHRTLAQRQADFQRPPGAAEPEPKDGGLQRRSSPAADVQGENF
CAAVKNTQPEDGVEMDTRQSPHDEDPQAVTYAKVKHSRPRREMASPPSP
LSGEFLDTKDRQAEEDRQMDTEAAASEAPQDVTYAQLHSFTLRQKATEP
PPSQEGASPAEPSVYATLAIH.

In some embodiments, the LILRB4 domain comprises an amino acid sequence having at least 85% identity, at least 90% identity, at least 95% identity, at least 96% identity, at least 97% identity, at least 98% identity, at least 99% identity or is identical to a fragment of SEQ ID NO: 126.

In some embodiments, the inhibitory domain comprises a LILRB5 domain or a functional fragment thereof. Inclusion of the LILRB5 domain in the engineered TCR can inhibit T cell activation. In some embodiments, LILRB5 comprises an amino acid sequence having at least 85% identity, at least 90% identity, at least 95% identity, at least 96% identity, at least 97% identity, at least 98% identity, at least 99% identity or is identical to a sequence of.

(SEQ ID NO: 127)
RHRHQSKHRTSAHFYRPAGAAGPEPKDQGLQKRASPVADIQEEILNAAV
KDTQPKDGVEMDARAAASEAPQDVTYAQLHSLTLRREATEPPPSQEREP
PAEPSIYAPLAIH.

In some embodiments, the LILRB5 domain comprises an amino acid sequence having at least 85% identity, at least 90% identity, at least 95% identity, at least 96% identity, at least 97% identity, at least 98% identity, at least 99% identity or is identical to a fragment of SEQ ID NO: 127.

In some embodiments, the inhibitory domain comprises a PD-1 (also referred to herein as PD1) domain or a functional fragment thereof. Inclusion of the PD-1 domain in the engineered TCR can inhibit T cell activation. In some embodiments, PD-1 comprises an amino acid sequence having at least 85% identity, at least 90% identity, at least 95% identity, at least 96% identity, at least 97% identity, at least 98% identity, at least 99% identity or is identical to a sequence of:

| (SEQ ID NO: 128) | |
|---|---|
| 1 | MQIPQAPWPV VWAVLQLGWR PGWFLDSPDR PWNPPTFSPA LLVVTEGDNA TFTCSFSNTS |
| 61 | ESFVLNWYRM SPSNQTDKLA AFPEDRSQPG QDCRFRVTQL PNGRDFHMSV VRARRNDSGT |
| 121 | YLCGAISLAP KAQIKESLRA ELRVTERRAE VPTAHPSPSP RPAGQFQTLV VGVVGGLLGS |
| 181 | LVLLVWVLAV ICSRAARGTI GARRTGQPLK EDPSAVPVFS VDYGELDFQW REKTPEPPVP |
| 241 | CVPEQTEYAT IVFPSGMGTS SPARRGSADG PRSAQPLRPE DGHCSWPL. |

In some embodiments, the PD-1 domain comprises an amino acid sequence having at least 85% identity, at least 90% identity, at least 95% identity, at least 96% identity, at least 97% identity, at least 98% identity, at least 99% identity or is identical to a fragment of SEQ ID NO: 128.

In some embodiments, the PD-1 intracellular domain comprises an amino acid sequence having at least 85% identity, at least 90% identity, at least 95% identity, at least 96% identity, at least 97% identity, at least 98% identity, at least 99% identity or is identical to a sequence of:

| (SEQ ID NO: 129) | |
|---|---|
| 1 | TERRAEVPTA HPSPSPRPAG QFQTLVVGVV GGLLGSLVLL VWVLAVICSR AARGTIGARR |
| 61 | TGQPLKEDPS AVPVFSVDYG ELDFQWREKT PEPPVPCVPE QTEYATIVFP SGMGTSSPAR |
| 121 | RGSADGPRSA QPLRPEDGHC SWPL. |

In some embodiments, the PD-1 intracellular domain comprises or consists essentially of SEQ ID NO: 129.

In some embodiments, the PD-1 intracellular domain comprises an amino acid sequence having at least 85% identity, at least 90% identity, at least 95% identity, at least 96% identity, at least 97% identity, at least 98% identity, at least 99% identity or is identical to a sequence of:

(SEQ ID NO: 130)
RAARGTIGARRTGQPLKEDPSAVPVFSVDYGELDFQWREKTPEPPVPCV
PEQTEYATIVFPSGMGTSSPARRGSADGPRSAQPLRPEDGHCSWPL.

In some embodiments, the PD-1 intracellular domain comprises or consists essentially of SEQ ID NO: 130.

In some embodiments, the inhibitory domain comprises a CTLA-4 domain or a functional fragment thereof. Inclusion of the CTLA-4 domain in the engineered TCR can inhibit T cell activation. In some embodiments, CTLA-4 comprises an amino acid sequence having at least 85% identity, at least 90% identity, at least 95% identity, at least 96% identity, at least 97% identity, at least 98% identity, at least 99% identity or is identical to a sequence of:

| (SEQ ID NO: 131) | |
|---|---|
| 1 | MACLGFQRHK AQLNLATRTW PCTLLFFLLF IPVFCKAMHV AQPAVVLASS RGIASFVCEY |
| 61 | ASPGKATEVR VTVLRQADSQ VTEVCAATYM MGNELTFLDD SICTGTSSGN QVNLTIQGLR |
| 121 | AMDTGLYICK VELMYPPPYY LGIGNGTQIY VIDPEPCPDS DFLLWILAAV SSGLFFYSFL |
| 181 | LTAVSLSKML KKRSPLTTGV YVKMPPTEPE CEKQFQPYFI PIN. |

In some embodiments, the CTLA-4 domain comprises an amino acid sequence having at least 85% identity, at least 90% identity, at least 95% identity, at least 96% identity, at least 97% identity, at least 98% identity, at least 99% identity or is identical to a fragment of SEQ ID NO: 131.

In some embodiments, the inhibitory domain comprises a NKG2D domain or a functional fragment thereof. Inclusion of the NKG2D domain in the engineered TCR can inhibit T cell activation. In some embodiments, NKG2D comprises an amino acid sequence having at least 85% identity, at least 90% identity, at least 95% identity, at least 96% identity, at least 97% identity, at least 98% identity, at least 99% identity or is identical to a sequence of:

| (SEQ ID NO: 132) | |
|---|---|
| 1 | MGWIRGRRSR HSWEMSEFHN YNLDLKKSDF STRWQKQRCP VVKSKCRENA SPFFFCCFIA |
| 61 | VAMGIRFIIM VAIWSAVFLN SLFNQEVQIP LTESYCGPCP KNWICYKNNC YQFFDESKNW |
| 121 | YESQASCMSQ NASLLKVYSK EDQDLLKLVK SYHWMGLVHI PTNGSWQWED GSILSPNLLT |
| 181 | IIEMQKGDCA LYASSFKGYI ENCSTPNTYI CMQRTV. |

In some embodiments, the NKG2D domain comprises an amino acid sequence having at least 85% identity, at least 90% identity, at least 95% identity, at least 96% identity, at least 97% identity, at least 98% identity, at least 99% identity or is identical to a fragment of SEQ ID NO: 59.

In some embodiments, the inhibitory domain comprises a FcgRIIB domain or a functional fragment thereof. Inclusion of the FcgRIIB domain in the engineered TCR can inhibit T cell activation. In some embodiments, FcgRIIB comprises an amino acid sequence having at least 85% identity, at least 90% identity, at least 95% identity, at least 96% identity, at least 97% identity, at least 98% identity, at least 99% identity or is identical to a sequence of

| (SEQ ID NO: 133) | |
|---|---|
| 1 | MGILSFLPVL ATESDWADCK SPQPWGHMLL WTAVLFLAPV AGTPAAPPKA VLKLEPQWIN |
| 61 | VLQEDSVTLT CRGTHSPESD SIQWFHNGNL IPTHTQPSYR FKANNNDSGE YTCQTGQTSL |
| 121 | SDPVHLTVLS EWLVLQTPHL EFQEGETIVL RCHSWKDKPL VKVTFFQNGK SKKFSRSDPN |
| 181 | FSIPQANHSH SGDYHCTGNI GYTLYSSKPV TITVQAPSSS PMGIIVAVVT GIAVAAIVAA |
| 241 | VVALIYCRKK RISALPGYPE CREMGETLPE KPANPTNPDE ADKVGAENTI TYSLLMHPDA |
| 301 | LEEPDDQNRI. |

In some embodiments, the FcgRIIB domain comprises an amino acid sequence having at least 85% identity, at least 90% identity, at least 95% identity, at least 96% identity, at least 97% identity, at least 98% identity, at least 99% identity or is identical to a fragment of SEQ ID NO: 133.

In some embodiments, the inhibitory intracellular domain comprises an amino acid sequence having at least 85% identity, at least 90% identity, at least 95% identity, at least 96% identity, at least 97% identity, at least 98% identity, at least 99% identity or is identical to a sequence of:

(SEQ ID NO: 134)
RVKFSRSADAPAYQQPDPQEVTYAQLDKRRGRDPEMGGKPRRKPDPQEV
TYAQLGMKGERRRGKGHPDPQEVTYAOLHMOALPPR.

In some embodiments, the inhibitory intracellular domain comprises or consists essentially of SEQ ID NO: 134.

In some embodiments the inhibitory receptor comprises 2 or more inhibitory intracellular domains.

### Nucleic Acids and Vectors

The present disclosure provides nucleic acids encoding the polypeptides comprising HLA-A*11 antigen binding domains, antibodies and receptors as disclosed herein. The present disclosure also provides vectors in which a nucleic acid encoding the antigen binding domain, antibody, receptor or receptor subunit is inserted. Vectors derived from retroviruses are suitable tools to achieve long-term gene transfer since they allow for genetic stability and high expression, in addition to having a flexible genome. Furthermore, clinical experience with retroviral vectors provides guidance for optimizing efficacy and safety in their use.

In brief summary, the expression of natural or synthetic nucleic acids encoding a receptor is typically achieved by operably linking a nucleic acid encoding the receptor or a portion thereof to a promoter, and incorporating the construct into an expression vector. The vectors can be suitable for replication and integration in eukaryotes. Typical cloning vectors contain transcription and translation terminators, initiation sequences, and promoters useful for regulation of the expression of the desired nucleic acid sequence.

The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors. Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, gammaretroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, adeno-associated virus vectors are used. In some embodiments, retrovirus vectors are used.

Additional promoter elements, e.g., enhancers, regulate the frequency of transcriptional initiation. In some embodiments, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. Depending on the promoter, it appears that individual elements can function either cooperatively or independently to activate transcription.

Various promoter sequences may be used, including, but not limited to the immediate early cytomegalovirus (CMV) promoter, Elongation Growth Factor-1α (EF-1α), simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of the polypeptides of the disclosure, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20 degrees Celsius. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Cells

The disclosure provides cells comprising the nucleic acids and vectors of the disclosure, or expressing the antibodies, antigen binding domains or receptors comprising same of the disclosure. Also provided are populations of said cells, and compositions comprising the populations of cells. Among the compositions are pharmaceutical compositions and formulations for administration, such as for adoptive cell therapy. Also provided are therapeutic methods for administering the cells and compositions to subjects, e.g., patients.

The disclosure provides cells expressing receptors, such as CARs or TCRs, specific to HLA-A*11. Cells of the disclosure may also comprise one or more additional exogenous receptor, in addition to the HLA-A*11 specific receptor. For example, a cell of the disclosure may comprise both an inhibitory CAR or TCR specific to HLA-A*11, and a second CAR or TCR specific to a cancer antigen that is an activator receptor. The cell expressing this two receptor system can be used in an adoptive cell therapy targeting cancer cells that express the cancer antigen, but have lost HLA-A*11 due to loss of heterozygosity. Expression of the HLA-A*11 inhibitory receptor increases the specificity of the adoptive cell therapy for the cancer cells, as normal, non-cancer cells that express both the cancer antigen and HLA-A*11 will not activate the adoptive immune cells. In some embodiments, the cells expressing an HLA-A*11 inhibitory receptor further express an activator receptor specific to a cancer antigen, whereby an activating signal delivered through the activator receptor is diminished or inhibited by binding of the inhibitory receptor to the HLA-A*11 antigen.

In some embodiments, the HLA-A*11 specific receptor expressing cells of the present invention may further comprise one or more additional activator receptors that target an antigen selected from the group of BCMA, BCR-Ab1, BST2, CAIX, CD19, CD20, CD22, CD123, CD171, CD30, CD33, CD38, CD44v6, CD44v7/8, CEA, CLL-1, EGFR, EGFRvIII, EGP-2, EGP-40, ERBB2 (Her2/neu), EPCAM, fetal acetylcholine receptor, FBP, FLT3, Folate receptor alpha, GD2, GD3, Her3 (ErbB3), Her4 (ErbB4), k-light chain, KDR, MAD-CT-1, MAD-CT-2, MAGE-A1, MAGE-A3, MART1, ML-IAP, MYCN, Oncofetal antigen (h5T4), NKG2D ligands PDKI, PDL1, PSCA, PSMA, PRSS21, RORI, SLAMF7, TAG-72, Tn Ag, TSLPR, B7H3 (CD276), KIT (CD17), IL-13Ra2, Mesothelin, IL-11Ra, VEGFR2, LeY, CD24, PDGFR-beta, SSEA-4; CD20, MUC1, EGFR, NCAM, Prostase, PAP, ELF2M, Ephrin B2, FAP, IGF-1 receptor, CAFX, LMP2, gp100, tyrosinase, EphA2, Fucosyl GM1, sLe, ganglioside GM3, TGS5, HMWMAA, OAcGD2, OR51E2, Folate receptor beta, TEM1/CD248, TEM7R, CLDN6, TSHR, GloboH, GPR20, GPRC5D, CXORF61, CD97, CD179a, ADRB3, ALK, Polysialic acid, PANX3, PLAC1, NY-BR-1, NY-ESO-1, UPK2, TIM-1, HAVCR1, LY6K, TARP, WT1, LAGE-1a, ETV6-AML, SPA17, XAGE1, Tie 2, Fos-related antigen 1, p53, p53 mutant, prostein, survivin, telomerase, PCTA-1, Rat sarcoma Ras mutant, hTERT, sarcoma translocation breakpoints, ERG, NA17, PAX3, Androgen receptor, Cyclin B1, RhoC, TRP-2, CYP1B1, BORIS, SART3, PAX5, OY-TES1, LCK, AKAP-4, SSX2, RAGE-1, RU1, RU2, legumain, HPV E6, HPV E7, intestinal carboxyl esterase, mut hsp70-2, CD79a, CD79b, CD72, LAIR1, CD89, LILRA2, CD300LF, CLEC12A, EMR2, FCRL5, GPC3, IGLL1, and LY75.

The cells of the disclosure generally are eukaryotic cells, such as mammalian cells, and typically are human cells, more typically primary human cells, e.g., allogeneic or autologous donor cells. The cells may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. In some embodiments, the cells are derived from the blood, bone marrow, lymph, or lymphoid organs, are cells of the immune system, such as cells of the innate or adaptive immune systems, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation.

In some embodiments, the cell expressing the HLA-A*11 specific receptor is a recombinant immune cell. In some embodiments, the recombinant immune cell is a T cell or an NK cell.

With reference to the subject to be treated, the cells may be allogeneic and/or autologous. Among the methods included are off-the-shelf methods. In some aspects, such as for off-the-shelf technologies, the cells are pluripotent and/or multipotent, such as stem cells, induced pluripotent stem cells (iPSCs), or T cells that either lack or are engineered to be deficient in T cell receptor function. In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4⁺ and/or of CD8⁺ T cells are naïve T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells, Natural Killer T (NKT) cells, cytokine-induced killer (CIK) cells, tumor-infiltrating lymphocytes (TIL), lymphokine-activated killer (LAK) cells, or the like. In some embodiments, the cells are monocytes or granulocytes, e.g., myeloid cells, macrophages, neutrophils, dendritic cells, mast cells, eosinophils, and/or basophils.

In some embodiments, the cells are derived from cell lines, e.g., T cell lines. The cells in some embodiments are obtained from a xenogeneic source, for example, from mouse, rat, non-human primate, and pig.

Cells expressing HLA-A*11 specific receptors may further comprise a disruption to one or more endogenous genes. In some embodiments, the endogenous gene encodes TCRα, TCRβ, CD52, glucocorticoid receptor (GR), deoxycytidine kinase (dCK), or an immune checkpoint protein such as, for example, programmed death-1 (PD-1).

The HLA-A*11 specific receptors of the instant disclosure, and cells expressing these receptors may be further modified to improve efficacy against solid tumors. This increased efficacy may be measured by an increase in tumor cytotoxicity, tumor infiltration, and evasion of or resistance to tumor immunosuppressive mediators. In some embodiments, enhanced anti-tumor efficacy may be characterized by increased TCR signaling, increased cytokine release, enhanced killing of tumor cells, increased T cell infiltration of established tumors, improved tumor trafficking, attenuated tumor-induced hypofunction, and improved migration and chemotaxis.

In one aspect, the cells expressing HLA-A*11 specific receptors are further modified to evade or neutralize the activity of immunosuppressive mediators, including, but not limited to prostaglandin E2 (PGE2) and adenosine. In some embodiments, this evasion or neutralization is direct. In other embodiments, this evasion or neutralization is mediated via the inhibition of protein kinase A (PKA) with one or more binding partners, for example ezrin. In specific embodiments, the cells expressing HLA-A*11 specific receptors further express the peptide “regulatory subunit I anchoring disruptor” (RIAD). RIAD is thought to inhibit the association of protein kinase A (PKA) with ezrin, which thus prevents PKA-mediated inhibition of TCR activation (Newick et al. Cancer Res 2016 August; 76(15 Suppl): Abstract nr B27).

In some embodiments, the cells expressing HLA-A*11 specific receptors may induce a broad antitumor immune response consistent with epitope spreading.

In some embodiments, the cells expressing HLA-A*11 specific receptors further comprise a homing mechanism. For example, the cell may transgenically express one or more stimulatory chemokines or cytokines or receptors thereof. In particular embodiments, the cells are genetically modified to express one or more stimulatory cytokines. In certain embodiments, one or more homing mechanisms are used to render the inventive cells resistant to an inhibitory tumor microenvironment. In some embodiments, the cells expressing HLA-A*11 specific receptors are further modified to release inducible cytokines upon receptor activation, e.g., to attract or activate innate immune cells to a targeted tumor (so-called fourth generation CARs or TRUCKS). In some embodiments, CARs may co-express homing molecules, e.g., CCR4 or CCR2b, to increase tumor trafficking.

### Regulation of Expression

In some instances, it may be advantageous to regulate the activity of the HLA-A*11 specific receptor or cells expressing the HLA-A*11 specific receptor. For example, inducing apoptosis using, e.g., a caspase fused to a dimerization domain (see, e.g., Di et al., N Engl. J. Med. 2011 Nov. 3; 365(18):1673-1683), can be used as a safety switch in the adoptive cell therapy of the instant disclosure. In another example, HLA-A*11 specific receptor expressing cells can also express an inducible Caspase-9 (iCaspase-9) molecule that, upon administration of a dimerizer drug (e.g., rimiducid (also called AP1903 (Bellicum Pharmaceuticals) or AP20187 (Ariad)) leads to activation of the Caspase-9 and apoptosis of the cells. The iCaspase-9 molecule contains a chemical inducer of dimerization (CID) binding domain that mediates dimerization in the presence of a CID. This results in inducible and selective depletion of HLA-A*11 specific receptor expressing cells. In some cases, the iCaspase-9 molecule is encoded by a nucleic acid molecule separate from the receptor encoding vector(s). In some cases, the iCaspase-9 molecule is encoded by the same nucleic acid molecule as the receptor encoding vector. The iCaspase-9 can provide a safety switch to avoid any toxicity of receptor expressing cells. See, e.g., Song et al. Cancer Gene Ther. 2008; 15(10):667-75; Clinical Trial Id. No. NCT02107963; and Di Stasi et al. N. Engl. J. Med. 2011; 365:1673-83.

Alternative strategies for regulating the adoptive cell therapy of the instant disclosure include utilizing small molecules or antibodies that deactivate or turn off receptor activity, e.g., by deleting receptor expressing cells, e.g., by inducing antibody dependent cell-mediated cytotoxicity (ADCC). For example, HLA-A*11 specific receptor expressing cells described herein may also express an antigen that is recognized by molecules capable of inducing cell death, e.g., ADCC or compliment-induced cell death. For example, HLA-A*11 specific receptor expressing cells described herein may also express a receptor capable of being targeted by an antibody or antibody fragment. Examples of such receptors include EpCAM, VEGFR, integrins (e.g., integrins αvβ3, α4, αI3/4β3, α4β7, α5β1, αvβ3, αv), members of the TNF receptor superfamily (e.g., TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/lgE Receptor, CD25, CD28, CD30, CD33, CD3ε, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (e.g., versions preserving one or more extracellular epitopes but lacking one or more regions within the cytoplasmic domain). For example, HLA-A*11 specific receptor expressing cells described herein may also express a truncated epidermal growth factor receptor (EGFR) which lacks signaling capacity but retains the epitope that is recognized by molecules capable of inducing ADCC, e.g., cetuximab (ERBITUX®), such that administration of cetuximab induces ADCC and subsequent depletion of the HLA-A*11 specific receptor expressing cells (see, e.g., WO2011/056894, and Jonnalagadda et al., Gene Ther. 2013; 20(8)853-860).

In some embodiments, the HLA-A*11 specific receptor expressing cell comprises a polynucleotide encoding a suicide polypeptide, such as for example RQR8. See, e.g., WO2013153391A, which is hereby incorporated by reference in its entirety. In HLA-A*11 specific receptor expressing cells comprising the polynucleotide, the suicide polypeptide may be expressed at the surface of the cell. The suicide polypeptide may also comprise a signal peptide at the amino terminus. Another strategy includes expressing a highly compact marker/suicide gene that combines target epitopes from both CD32 and CD20 antigens in the HLA-A*11 specific receptor expressing cells described herein, which binds rituximab, resulting in selective depletion of the HLA-A*11 specific receptor expressing cells, e.g., by ADCC (see, e.g., Philip et al., Blood. 2014; 124(8)1277-1287). Other methods for depleting the cells described herein include administration of CAMPATH, a monoclonal anti-CD52 antibody that selectively binds and targets mature lymphocytes, e.g., HLA-A*11 specific receptor expressing lymphocytes, for destruction, e.g., by inducing ADCC. In other embodiments, the HLA-A*11 specific receptor expressing cell can be selectively targeted using a receptor ligand, e.g., an anti-idiotypic antibody. In some embodiments, the anti-idiotypic antibody can cause effector cell activity, e.g., ADCC or ADC activities, thereby reducing the number of cells. In other embodiments, the receptor ligand, e.g., the anti-idiotypic antibody, can be coupled to an agent that induces cell killing, e.g., a toxin, thereby reducing the number of cells. Alternatively, the receptors themselves can be configured such that the activity can be regulated, e.g., turned on and off as described below.

In some embodiments, the HLA-A*11 specific receptor expressing immune cell may only transiently express the receptor. For example, the cells of the disclosure may be transduced with mRNA comprising a nucleic acid sequence encoding an HLA-A*11 specific receptor or subunit thereof. In this vein, the present disclosure also includes RNA constructs that can be directly transfected into a cell. Methods for generating mRNA for use in transfection involves in vitro transcription (IVT) of a template, followed by polyA addition, to produce a construct containing 3′ and 5′ untranslated sequences (“UTRs”), a 5′ cap and/or Internal Ribosome Entry Site (IRES), the nucleic acid to be expressed, and a polyA tail, typically 50-2000 bases in length. RNA so produced can efficiently transfect different kinds of cells. In some embodiments, the template includes sequences for the HLA-A*11 specific receptor or subunit thereof. In some embodiments, an RNA vector encoding the HLA-A*11 specific receptor or subunit thereof is transduced into a cell by electroporation.

In some embodiments, the antigen-binding domain of the HLA-A*11 receptor is affinity tuned. This may be accomplished, e.g., through the use of a HLA-A*11 receptor expressing T cell with target antigen affinities varying over three orders of magnitude (Liu et al. Cancer Res 2015 September; 75(17):3596-607). Additionally, in vivo xenograft models may be used to evaluate the toxicity of affinity tuned receptors on normal human tissue (Johnson et al. Sci Transl Med 2015 February; 7(275):275ra22).

In some embodiments, the HLA-A*11 receptor expressing cells of the invention may be further genetically modified to express the dominant negative form of the transforming growth factor (TGF) beta receptor (DNR).

In another embodiment, the HLA-A*11 receptor expressing cell may be specific for another antigen, including a tumor antigen in some cases. In some embodiments, the transformed host cells may be selected for specificity for one or more strong viral antigens or may be transformed to exhibit specificity for these antigens. In specific embodiments, the cells are pp65CMV-specific T cells, CMV-specific T cells, EBV-specific T cells, Varicella Virus-specific T cells, Influenza Virus-specific T cells and/or Adenovirus-specific T cells.

To increase persistence, the cells of the disclosure may be further modified to overexpress pro-survival signals, reverse anti-survival signals, overexpress Bcl-xL, overexpress hTERT, lack Fas, or express a TGFβ dominant negative receptor. Persistence may also be facilitated by the administration of cytokines, e.g., IL-2, IL-7, and IL-15.

### Methods of Use

The disclosure provides methods of using the disclosed anti-HLA-A*11 antibodies, antigen binding domains, receptors and cells comprising same for the treatment of cancer.

In some embodiments, the methods for treating a cancer a subject in need thereof comprise administering a therapeutically effective amount of the recombinant immune cells comprising the HLA-A*11 specific receptors, or the pharmaceutical compositions comprising same, to the subject. In some embodiments, for example those embodiments where the HLA-A*11 specific receptor is an inhibitory receptor, and the recombinant immune cells comprise an activator receptor specific to a tumor antigen, cells of the cancer have lost expression of HLA-A*11 due to loss of heterozygosity.

In some embodiments, the methods for treating cancer in a subject further comprise (a) determining if the subject is heterozygous for an HLA-A*11 allele; (b) isolating a plurality of cancer cells from the subject; (c) detecting the presence or absence of HLA-A*11 on the cancer cells using the HLA-A*11 specific antibodies or antigen binding domains described herein; and (d) administering the recombinant cell or pharmaceutical composition when the plurality of cancer cells do not express HLA-A*11.

The disclosure provides methods for determining whether cancer cells express HLA-A*11, comprising: (a) providing a plurality of cancer cells; and (b) detecting the presence of absence of HLA-A*11 on the cancer cells using the polypeptide comprising an HLA-A*11 specific antigen binding domain as described herein.

Thus, use of anti-HLA-A*11 antibodies and antigen binding domains in characterization of, and diagnosis of cancers is envisaged as within the scope of the instant disclosure. In some embodiments, the compositions of the present disclosure may be used in diagnostic methods to detect and/or quantify the presence of HLA-A*11 expressing cells. For example, in a subject with a cancer with loss of heterozygosity, or that is suspected to have loss of heterozygosity, the HLA-A*11 antibodies or antigen binding fragments thereof can be used to determine whether, or to what extent, cells of the cancer have lost HLA-A*11 expression.

The disclosure provides methods for determining the susceptibility of a cancer to an adoptive cell therapy comprising: (a) providing a plurality of cancer cells; and (b) detecting the presence of absence of HLA-A*11 on the cancer cells using the polypeptide comprising an HLA-A*11 specific antigen binding domain as described herein. In some embodiments, a plurality the cancer cells have lost expression of HLA-A*11. In some embodiments, for examples those embodiments where a plurality the cancer cells have lost expression of HLA-A*11, the methods further comprise administering an adoptive cell therapy. In some embodiments, the adoptive cell therapy comprises administering a plurality of immune cells comprising a first activator receptor specific to a cancer antigen expressed by the cancer cells, and a second inhibitory receptor specific to HLA-A*11.

### Diseases

Cancers that may be treated include tumors solid tumors and liquid tumors. Solid tumors include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the CARs of the invention include, but are not limited to, solid tumors, epithelial cancers, and hematological malignancies. In some embodiments, the cancer is a hematologic malignancy, multiple myeloma, an epithelial cancer, a solid tumor, melanoma, head and neck cancer, breast cancer, lung cancer, or synovial sarcoma. In some embodiments the cancer may be selected from a carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leciomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

In some embodiments, the adoptive cells of the invention are used to treat a cancer that has lost expression of HLA-A*11. Any cancers which have lost HLA-A*11, for example through loss of heterozygosity, are envisaged as within the scope of the instant disclosure. Cancers that have lost HLA-A*11 through loss of heterozygosity (LOH) can be treated using adoptive cell therapies employing a two-receptor system, which is described in WO 2021/030149, the contents of which are incorporated by reference in their entirety herein. In brief, adoptive immune cells express two receptors. The first receptor acts to activate, or promote activation of the immune cells, while the second receptor acts to inhibit activation by the first receptor. Differential expression of ligands for the first and second receptors, for example through loss of heterozygosity of the locus encoding the inhibitory ligand (in this case HLA-A*11), mediates activation of immune cells by target cells that express the first activator ligand but not the second inhibitory ligand.

In general, HLA-A*11 negative tumor cells may be identified via known methods, which are described in further detail below. For example, HLA-A*11 negative tumor cells may be identified via a lack of immunofluorescence when stained using anti-HLA-A*11 antibodies of the disclosure.

### Subjects

The subject referred to herein may be any living subject. In some embodiments, the subject is a mammal. The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes).

In some embodiments, the subject, to whom the cells, cell populations, or compositions are administered is a primate, such as a human. In some embodiments, the primate is a monkey or an ape. The subject can be male or female and can be any suitable age, including infant, juvenile, adolescent, adult, and geriatric subjects. In some examples, the patient or subject is a validated animal model for disease, adoptive cell therapy, and/or for assessing toxic outcomes such as cytokine release syndrome (CRS).

In some embodiments, the subject has persistent or relapsed disease, e.g., following treatment with another immunotherapy and/or other therapy, including chemotherapy, radiation, and/or hematopoietic stem cell transplantation (HSCT), e.g., allogenic HSCT. In some embodiments, the administration effectively treats the subject despite the subject having become resistant to another therapy. In some embodiments, the subject has not relapsed but is determined to be at risk for relapse, such as at a high risk of relapse, and thus the compound or composition is administered prophylactically, e.g., to reduce the likelihood of or prevent relapse.

In some embodiments, the methods include administration of HLA-A*11 specific receptor expressing cells or a composition containing the cells to a subject with a disease or disorder. In some embodiments, the cells, populations, and compositions are administered to a subject having the particular disease or condition to be treated, e.g., via adoptive cell therapy, such as adoptive T cell therapy. In some embodiments, the cells or compositions are administered to the subject, such as a subject having or at risk for the disease or condition. In some aspects, the methods thereby treat, e.g., ameliorate one or more symptom of the disease or condition, such as by lessening tumor burden in a subject with cancer.

### Dosage and Administration

The compositions of the present invention may be administered in a number of ways depending upon whether local or systemic treatment is desired.

In general, administration may be topical, parenteral, or enteral.

In the case of adoptive cell therapy, methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

The compositions of the invention are suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intratumoral, intrasynovial injection or infusions; and kidney dialytic infusion techniques. In some embodiments, parenteral administration of the compositions of the present invention comprises intravenous or intraarterial administration.

The disclosure provides pharmaceutical compositions comprising polypeptides comprising the disclosed anti-HLA-A*11 antigen binding domains, anti-HLA-A*11 receptors and cells comprising same, and a pharmaceutically acceptable carrier, diluent or excipient.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In some embodiments of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents, but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

The terms “oral”, “enteral”, “enterally”, “orally”, “non-parenteral”, “non-parenterally”, and the like, refer to administration of a compound or composition to an individual by a route or mode along the alimentary canal. Examples of “oral” routes of administration of a composition include, without limitation, swallowing liquid or solid forms of a composition from the mouth, administration of a composition through a nasojejunal or gastrostomy tube, intraduodenal administration of a composition, and rectal administration, e.g., using suppositories for the lower intestinal tract of the alimentary canal.

In some embodiments, the formulated composition comprising isolated anti-HLA-A*11 antibodies, antigen binding domains, or anti-HLA-A*11 expressing cells is suitable for administration via injection.

Pharmaceutical compositions and formulations for topical administration may include transdermal patches, ointments, lotions, creams, gels, drops, suppositories, sprays, liquids, semi-solids, monophasic compositions, multiphasic compositions (e.g., oil-in-water, water-in-oil), foams, microsponges, liposomes, nanoemulsions, aerosol foams, polymers, fullerenes, and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal, or intraventricular administration may include sterile aqueous solutions that may also contain buffers, diluents and other suitable additives such as, but not limited to, penetration enhancers, carder compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical compositions of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, aerosols, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In some embodiments of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Formulations comprising anti-HLA-A*11 antibodies or antigen binding domains, or anti-HLA-A*11 receptor expressing cells, may include pharmaceutically acceptable excipient(s). Excipients included in the formulations will have different purposes depending, for example, on the receptor construct, the subpopulation of cells used, and the mode of administration. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, and lubricating agents. The formulations comprising anti-HLA-A*11 antibodies or antigen binding domains, or anti-HLA-A*11 receptor expressing cells will typically have been prepared and cultured in the absence of any non-human components, such as animal serum (e.g., bovine serum albumin).

The formulation or composition may also contain more than one active ingredient useful for the particular indication, disease, or condition being treated with the binding molecules or cells, preferably those with activities complementary to the binding molecule or cell, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the pharmaceutically active agents or drugs may comprise immune checkpoint inhibitors, e.g., drugs that target PD-1, MAGE-A3, PD-L2, LAG3, CTLA4, KIR, CD244, B7-H3, B7-H4, BTLA, HVEM, GAL9, TIM3, and/or A2aR. Examples of these inhibitors include, but are not limited to, pidilizumab, nivolumab, pembrolizumab, atezolizumab, MDX-1105, BMS-936559, MED14736, MPDL3280A, MSB0010718C, tremelimumab, and ipilimumab, which may be administered alone or in combination with other agents, e.g., GM-CSF.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

Administration can be effected in one dose, continuously or intermittently throughout the course of treatment. Methods of determining the most effective means and dosage of administration are known to those of skill in the art and will vary with the composition used for therapy, the purpose of the therapy, the target cell being treated and the subject being treated. Single or multiple administrations can be carried out with the dose level and pattern being selected by the treating physician.

The pharmaceutical composition in some embodiments contains the anti-HLA-A*11 antibodies or antigen binding domains, or anti-HLA-A*11 receptor expressing cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The cells or population of cells can be administrated in one or more doses. In some embodiments, said effective amount of cells can be administrated as a single dose. In some embodiments, said effective amount of cells can be administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired. In some embodiments, an effective amount of cells or composition comprising those cells are administrated parenterally. In some embodiments, administration can be an intravenous administration. In some embodiments, administration can be directly done by injection within a tumor.

For purposes of the invention, the amount or dose of the adoptive cells administered should be sufficient to effect a therapeutic or prophylactic response in the subject or animal over a reasonable time frame. For example, the dose of the adoptive cells should be sufficient to bind to antigen, or detect, treat or prevent disease in a period of from about 2 hours or longer, e.g., about 12 to about 24 or more hours, from the time of administration. In certain embodiments, the time period could be even longer. The dose will be determined by the efficacy of the adoptive cells and the condition of the animal (e.g., human), as well as the body weight of the animal (e.g., human) to be treated.

For purposes of the invention, an assay, which comprises, for example, comparing the extent to which target cells are lysed or IFN-γ is secreted by T cells expressing the anti-HLA-A*11 receptor, polypeptide, or protein upon administration of a given dose of such T cells to a mammal, among a set of mammals of which is each given a different dose of the T cells, could be used to determine a starting dose to be administered to a mammal. The extent to which target cells are lysed or IFN-γ is secreted upon administration of a certain dose can be assayed by methods known in the art.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells or antibodies in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells or antibodies are administered prior to the one or more additional therapeutic agents. In some embodiments, the cells or antibodies are administered after to the one or more additional therapeutic agents.

In embodiments, a lymphodepleting chemotherapy is administered to the subject prior to, concurrently with, or after administration (e.g., infusion) of adoptive immune cells. In an example, the lymphodepleting chemotherapy is administered to the subject prior to administration of the cells. For example, the lymphodepleting chemotherapy ends 1-4 days (e.g., 1, 2, 3, or 4 days) prior to adoptive cell infusion. In embodiments, multiple doses of adoptive cells are administered, e.g., as described herein. In embodiments, a lymphodepleting chemotherapy is administered to the subject prior to, concurrently with, or after administration (e.g., infusion) of an anti-HLA-A*11 receptor expressing cell described herein. Examples of lymphodepletion include, but may not be limited to, nonmyeloablative lymphodepleting chemotherapy, myeloablative lymphodepleting chemotherapy, total body irradiation, etc. Examples of lymphodepleting agents include, but are not limited to, antithymocyte globulin, anti-CD3 antibodies, anti-CD4 antibodies, anti-CD8 antibodies, anti-CD52 antibodies, anti-CD2 antibodies, TCRαβ blockers, anti-CD20 antibodies, anti-CD19 antibodies, Bortezomib, rituximab, anti-CD 154 antibodies, rapamycin, CD3 immunotoxin, fludarabine, cyclophosphamide, busulfan, melphalan, Mabthera, Tacrolimus, alefacept, alemtuzumab, OKT3, OKT4, OKT8, OKT11, fingolimod, anti-CD40 antibodies, anti-BR3 antibodies, Campath-1H, anti-CD25 antibodies, calcineurin inhibitors, mycophenolate, and steroids, which may be used alone or in combination.

### Diagnostic Indications

The antibodies, antibody fragments and derivatives thereof of the disclosure are useful in methods known in the art relating to the localization and/or quantitation of a HLA-A*11 (e.g., for use in measuring levels of HLA-A*11 within appropriate physiological samples, for use in diagnostic methods, for use in imaging, and the like). The antibodies, antibody fragments and derivatives thereof of the disclosure are useful in isolating MHC complexes comprising HLA-A*11α chain by standard techniques, such as affinity chromatography or immunoprecipitation. An HLA-A*11 antibody of the disclosure can facilitate the purification of natural HLA-A*11α chain from biological samples, e.g., mammalian sera or cells as well as recombinantly-produced HLA-A*11α chain expressed in a host system. Moreover, HLA-A*11 antibodies can be used to detect an HLA-A*11α chain (e.g., in plasma, a cellular lysate or cell supernatant) in order to evaluate the abundance and pattern of expression of HLA-A*11α chain. The HLA-A*11 antibodies of the disclosure can be used diagnostically to monitor HLA-A*11 expression in tissue as part of a clinical testing procedure, e.g., to determine the efficacy of a given treatment regimen. The detection can be facilitated by coupling (i.e., physically linking) the HLA-A*11 antibody of this disclosure to a detectable substance.

The antibodies, antibody fragments or derivatives thereof of the disclosure are useful for detection of HLA-A*11α chain or MHC complexes comprising same. An exemplary method for detecting the level of HLA-A*11α chain or MHC complexes comprising same in a biological sample involves obtaining a biological sample from a subject and contacting the biological sample with an HLA-A*11 antibody of the present disclosure which is capable of detecting the HLA-A*11 polypeptide.

In one aspect, the HLA-A*11 antibodies, antibody fragments or derivatives thereof are detectably labeled. The term “labeled”, with regard to the antibody is intended to encompass direct labeling of the antibody by coupling (i.e., physically linking) a detectable substance to the antibody, as well as indirect labeling of the antibody by reactivity with another compound that is directly labeled. Non-limiting examples of indirect labeling include detection of a primary antibody using a fluorescently-labeled secondary antibody and end-labeling of a DNA probe with biotin such that it can be detected with fluorescently-labeled streptavidin.

The detection methods of this disclosure can be used to detect expression levels of HLA-A*11α chain or MHC complexes comprising same in a biological sample in vitro as well as in vivo. In vitro techniques for detection of HLA-A*11α chain or MHC complexes comprising same include enzyme linked immunosorbent assays (ELISAs), Western blots, flow cytometry, immunoprecipitations, radioimmunoassay, and immunofluorescence (e.g., IHC). Furthermore, in vivo techniques for detection of HLA-A*11α chain or MHC complexes comprising include introducing into a subject labeled anti-HLA-A*11 antibody. By way of example only, the antibody can be labeled with a radioactive marker whose presence and location in a subject can be detected by standard imaging techniques. In one aspect, the biological sample contains polypeptide molecules from the test subject.

Anti-HLA-A*11 antibodies, antibody fragments and derivatives thereof of the present disclosure can be used to assay HLA-A*11α chain levels in a biological sample (e.g., a cell or tissue sample) using antibody-based techniques. For example, protein expression in tissues can be studied with classical immunohistochemical (IHC) staining methods. Jalkanen, M. et al., J Cell. Biol. 101: 976-985 (1985); Jalkanen, M. et al., J Cell. Biol. 105: 3087-3096 (1987). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes or other radioactive agents, such as iodine (¹²⁵I, ¹²¹I, ¹³¹I), carbon (¹⁴C), sulfur (³⁵S), tritium (³H), indium (¹¹²In), and technetium (⁹⁹mTc), and fluorescent labels, such as fluorescein and rhodamine, and biotin.

In addition to assaying HLA-A*11α chain in a biological sample, HLA-A*11α chain levels can also be detected in vivo by imaging. Labels that can be incorporated with anti-HLA-A*11 antibodies for in vivo imaging of HLA-A*11α chain levels include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the HLA-A*11 antibody by labeling of nutrients for the relevant scFv clone.

An HLA-A*11 antibody which has been labeled with an appropriate detectable imaging moiety, such as a radioisotope (e.g., ¹³¹I, ¹¹²In, ⁹⁹mTc), a radio-opaque substance, or a material detectable by nuclear magnetic resonance, is introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled HLA-A*11 antibody will then preferentially accumulate at the location of cells which contain the specific target. For example, in vivo tumor imaging is described in S. W. Burchiel et al., Tumor Imaging: The Radiochemical Detection of Cancer 13 (1982).

In some aspects, anti-HLA-A*11 antibodies containing structural modifications that facilitate rapid binding and cell uptake and/or slow release are useful in in vivo imaging detection methods. In some aspects, the HLA-A*11 antibody contains a deletion in the CH2 constant heavy chain region of the antibody to facilitate rapid binding and cell uptake and/or slow release. In some aspects, a Fab fragment is used to facilitate rapid binding and cell uptake and/or slow release. In some aspects, a F(ab)′2 fragment is used to facilitate rapid binding and cell uptake and/or slow release.

The HLA-A*11 antibody compositions of the disclosure are useful in diagnostic and prognostic methods. As such, the present disclosure provides methods for using the antibodies of the disclosure useful in the diagnosis of HLA-A*11-related medical conditions in a subject. Antibodies of the disclosure may be selected such that they have a high level of epitope binding specificity and high binding affinity to HLA-A*11α chain. In general, the higher the binding affinity of an antibody, the more stringent wash conditions can be performed in an immunoassay to remove nonspecifically bound material without removing the target polypeptide. Accordingly, in some embodiments, HLA-A*11 antibodies of the disclosure useful in diagnostic assays have binding affinities of at least 10⁻⁶, 10⁻⁷, 10⁻⁸, 10⁻⁹, 10⁻¹⁰, 10⁻¹¹, or 10⁻¹² M. In certain aspects, HLA-A*11 antibodies used as diagnostic reagents have a sufficient kinetic on-rate to reach equilibrium under standard conditions in at least 12 hours, at least 5 hours, at least 1 hour, or at least 30 minutes.

Some methods of the disclosure employ polyclonal preparations of anti-HLA-A*11 antibodies and anti-HLA-A*11 antibody compositions of the disclosure as diagnostic reagents, and other methods employ monoclonal isolates. In methods employing polyclonal human anti-HLA-A*11 antibodies prepared in accordance with the methods described above, the preparation typically contains an assortment of HLA-A*11 antibodies, e.g., antibodies, with different epitope specificities to the target polypeptide. The monoclonal anti-HLA-A*11 antibodies of the present disclosure are useful for detecting a single antigen in the presence or potential presence of closely related antigens.

The HLA-A*11 antibodies of the present disclosure can be used as diagnostic reagents for any kind of biological sample. In one aspect, the HLA-A*11 antibodies disclosed herein are useful as diagnostic reagents for human biological samples. HLA-A*11 antibodies can be used to detect HLA-A*11α chain in a variety of standard assay formats. Such formats include immunoprecipitation, Western blotting, ELISA, radioimmunoassay, flow cytometry, IHC and immunometric assays. See Harlow & Lane, Antibodies, A Laboratory Manual (Cold Spring Harbor Publications, New York, 1988); U.S. Pat. Nos. 3,791,932; 3,839,153; 3,850,752; 3,879,262; 4,034,074, 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876. Biological samples can be obtained from any tissue (including biopsies), cell or body fluid of a subject.

The disclosure also provides for prognostic (or predictive) assays for determining whether a subject is at risk of developing a medical disease or condition associated with HLA-A*11. Such assays can be used for prognostic or predictive purpose to thereby prophylactically treat an individual prior to the onset of a medical disease or condition characterized by or associated with HLA-A*11. For example, HLA-A*11 is associated a variety of diseases, such as familial otosclerosis, pulmonary tuberculosis, leprosy, and cytomegalovirus infection with epilepsy. In addition, certain viruses, such as Epstein-Barr Virus, may be able to downregulate expression of HLA-A*11, which may contribute to the development of cancers such as lymphomas.

Another aspect of the disclosure provides methods for determining HLA-A*11 expression in a subject to thereby select appropriate therapeutic or prophylactic compounds for that subject.

Automated Embodiments. A person of ordinary skill in the art will appreciate that aspects of the methods for using the HLA-A*11 antibodies disclosed herein can be automated. Ventana Medical Systems, Inc. is the assignee of a number of United States patents disclosing systems and methods for performing automated analyses, including U.S. Pat. Nos. 5,650,327, 5,654,200, 6,296,809, 6,352,861, 6,827,901 and 6,943,029, and U.S. published application Nos. 20030211630 and 20040052685, each of which is incorporated herein by reference. Particular aspects of HLA-A*11 staining procedures can be conducted using various automated processes.

A biopsy is the removal of tissue and/or cells from an individual. Such removal may be to collect tissue and/or cells from the individual in order to perform experimentation on the removed tissue and/or cells, or diagnostic methods using the HLA-A*11 antibodies described herein. This experimentation may include experiments to determine if the individual has and/or is suffering from a certain condition or disease-state. The condition or disease may be, e.g., cancer. With respect to detecting the presence of HLA-A*11 negative tumor cells in a host, the sample comprising cells of the host can be a sample comprising whole cells, lysates thereof, or a fraction of the whole cell lysates, e.g., a nuclear or cytoplasmic fraction, a whole protein fraction, or a nucleic acid fraction. If the sample comprises whole cells, the cells can be any cells of the host, e.g., the cells of any organ or tissue, including blood cells or endothelial cells.

### Kits and Articles of Manufacture

The disclosure provides kits comprising polypeptides comprising HLA-A*11 antibodies, antigen binding fragments or derivatives thereof, receptors comprising same, or polynucleotides or vectors encoding same.

The disclosure provides kits comprising cells comprising anti-HLA-A*11 receptors. In some embodiments, the anti-HLA-A*11 receptors are inhibitory receptors, for example inhibitory receptors comprising a LILRB1 intracellular domain.

The disclosure provides kits comprising pharmaceutical compositions comprising cells comprising anti-HLA-A*11 receptors.

In some embodiments of the kits of the disclosure, the kits further comprise positive controls (e.g. HLA-A*11 antigen), negative controls, appropriate buffers and instructions for use.

### EXAMPLES

### Example 1: Generation and Screening of HLA-A*11 scFv Library

### HuTARG™ Sort

HuTARG™ primary libraries were from Innovative Targeting Solutions, Inc. An in vitro V(D)J repertoire with >1 billion diversity was generated by expression of RAG-1 and TdT in the host cells as described previously (U.S. Pat. No. 8,012,714; Oh et al. Sci. Rep. 9:17291 (2019)). pMHC probes were generated as described previously (Xu et al. Mol. Immunol. 125:56-64 (2020)). The library was enriched for cells displaying scFvs that bind specifically to target pMHC probes, but not to off-target pMHC probes using a flow sorter device. The library was simultaneously sorted for HLA-A*11 and three other HLA alleles. In rounds 1-3, the off-target was HLA-A*02, and the on-target was a combo of HLA-A*11 and the other 3 alleles. Then in the NGS round, each allele was sorted and sequenced separately. Multiple enrichment rounds were performed to increase on-target and decrease off-target binding. In the final round, on-target and off-target binding cells were collected. RNA was extracted from these pools and reverse transcribed into cDNA. PCR fragments containing the CDR regions were generated using the cDNAs as template, followed by targeted next generation sequencing (NGS) to determine the frequency of each binder with a unique CDR region. The degree of enrichment/depletion was determined by comparing the output and input NGS counts.

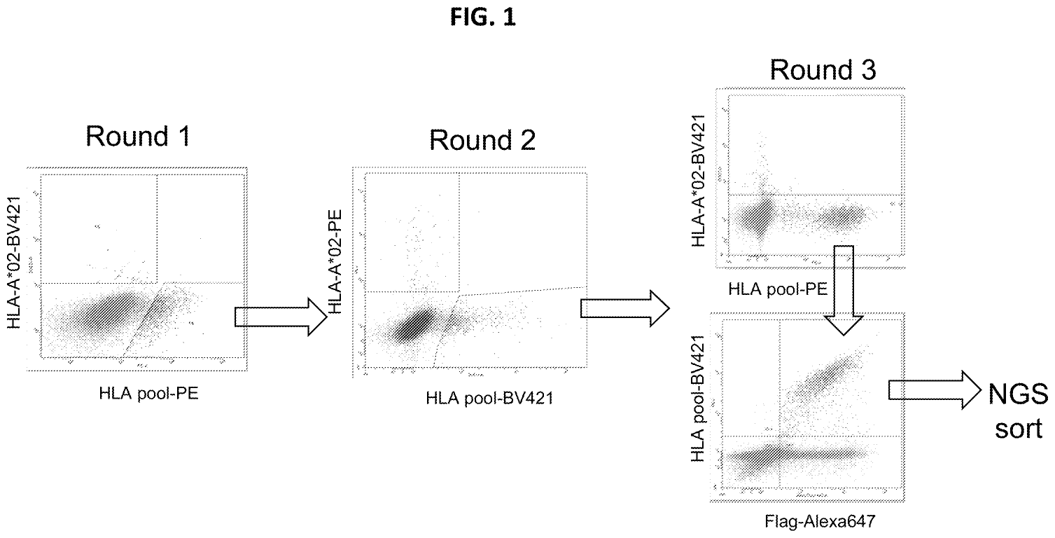

FIG. 1 shows the enrichment of the HLA-A*11 scFv HuTARG library through three rounds of fluorescence activated cell sorting (FACS), followed by NGS.

### Example 2: HLA-A*11 Chimeric Antigen Receptor (CAR) Activity in T2 Cells

### Cell Lines and Peptides

T2 cells were purchased from ATCC®. Jurkat NFAT-Firefly-Luciferase (JNL) cells were purchased from BPS Bioscience. All cell lines were cultured in the media as recommended by the vendors. HLA-A, -B and -C alleles were knocked out via CRISPR/Cas9 gene editing from T2 cells, which were subsequently transduced with lentivirus encoding HLA-A*11:01 at an MOI=5, and flow sorted for HLA class I expression using W6/32 antibody.

Peptide VVVGAVGVGK (SEQ ID NO: 110), with which the HLA-A*11 T2 cells were loaded, was purchased from GenScript.

### Molecular Cloning

CAR constructs were created by fusing an scFv LBD to a hinge, a transmembrane domain (TM) and an intracellular signaling domain (ICD). Sequences for scFv 1-9 are provided in Table 3. The hinge was derived from CD8, the TM from CD28, and the intracellular domain (ICD) from CD28, 4-1BB and CD3ζ. Gene segments were combined using Golden Gate cloning and inserted downstream of a human EF1alpha promoter contained in a lentivirus expression plasmid. Sequences of CAR and TCR constructs are shown in Table 6, below.

### Jurkat NFAT-Luciferase T2 Cell Assay

Jurkat NFAT-Luciferase effector cells (JNL) cells were transfected on day 1 with TCR or CAR constructs using standard protocols for the Neon™ transfection system (ThermoFisher MPK5000). T2 HLA-A*11 target cells were loaded with peptide VVVGAVGVGK (SEQ ID NO: 110). Peptides were resuspended in DMSO, and serially-diluted 20 times, 3× per dilution. Serially diluted peptide solutions were added to T2 HLA-A*11 cells resuspended in peptide-loading media (RPMI 1640+1% BSA+1×P/S). This yielded peptide-loaded T2 HLA-A*11 cells at approximately ˜0.67E6/mL, with peptide concentrations ranging from ˜10 fM to 100 μM, including a control at 0 μM. Peptide-loaded T2 HLA-A*11 cells were incubated overnight at 37° C. in 384-well plates, plated at 10,000 cells per well (Corning 3570). On day 2, transfected JNL cells (10,000 cells/well) were added to the peptide-loaded T2 HLA-A*11 cells to a final volume of 30 μL. After a 6-hour incubation at 37° C., the One-Step™ Luciferase assay system (Firefly luciferase, BPS Bioscience, 60690) was used to determine luminescence intensity on a Tecan Infinite® M1000.

### Flow Analysis

Protein L (ThermoFisher 29997) or an anti-murine TCRb (Biolegend 50-166-380) antibody was used to stain for surface expression of CAR or TCR, respectively, on JNL cells. HLA-A*11:01 pMHC tetramer was used to stain for ligand binding.

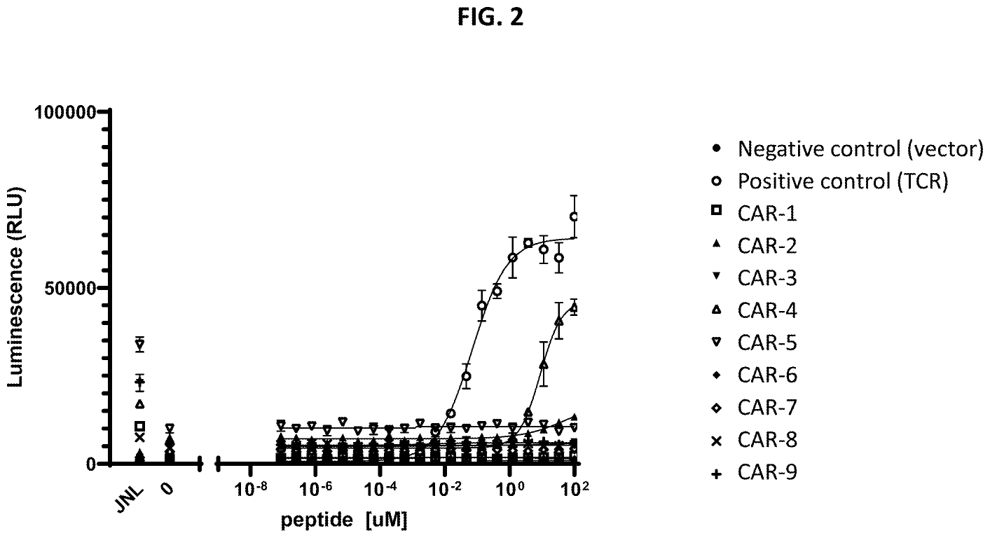

T2 cells were loaded with a serially diluted HLA-A*11-presented peptide as described above, and Jurkat NFAT-Luciferase cells were transiently transfected to express CARs or a positive control TCR. scFv sequences corresponding to CARs 1-9 are shown in Table 3. The functional response (RLU) was assessed after 6 hours of co-culture, and the results are shown in FIG. 2. As shown in FIG. 2 CAR-4 was able to activate Jurkat cells following co-culture with HLAA*QL T2 target cells.

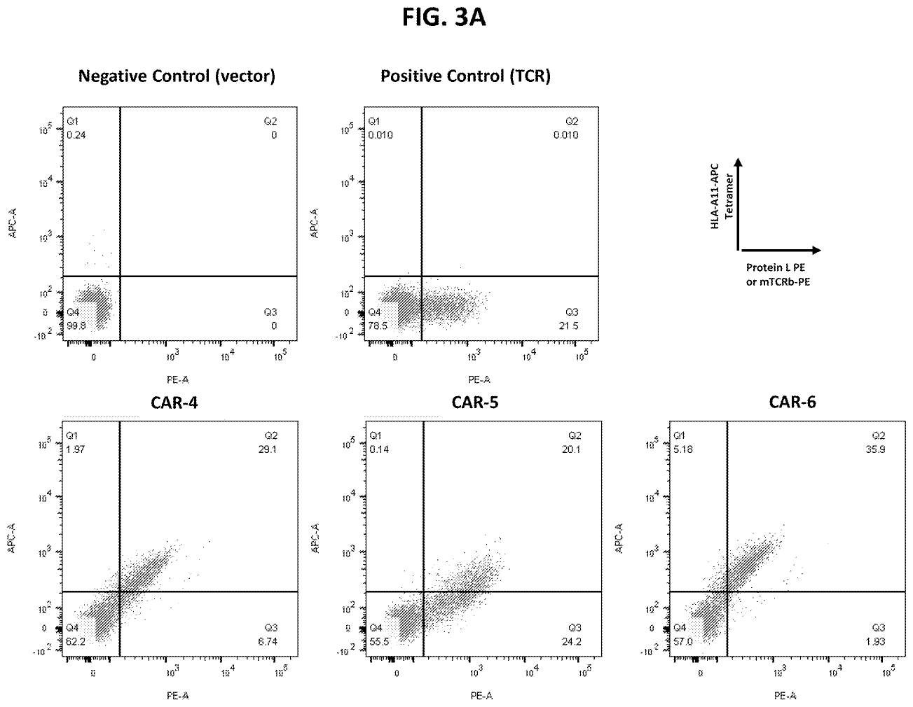

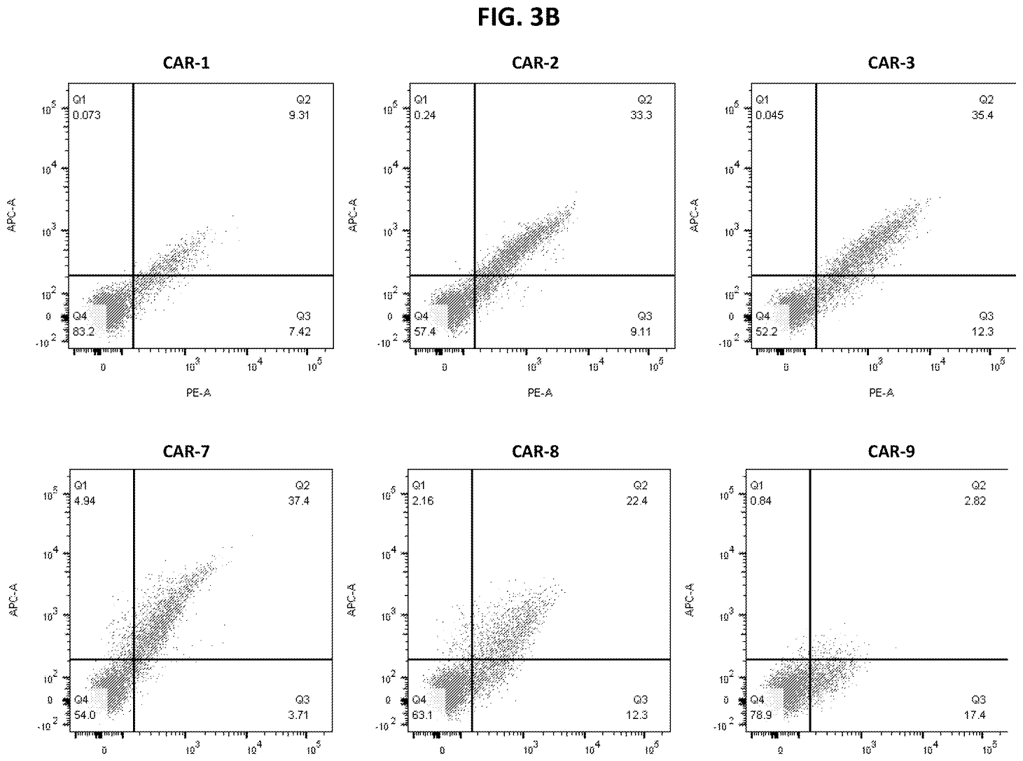

Expression of HLAA*11 CAR constructs by Jurkat NFAT-Luciferase cells is shown in FIGS. 3A-3B. Protein L or an anti-murine TCRb antibody was used to stain for CAR surface expression, and can be seen on the x-axis. An HLA-A*11 pMHC tetramer was used to stain for ligand binding, and is shown on the y-axis in FIGS. 3A-3B.

TABLE 6
Sequences of CAR constructs (anti-HLA-A*11 VH regions underlined)

| | | DNA |
|---|---|---|
| Name | Protein Sequence | Sequence |
| Positive | MKTVTGPLFLCFWLQLNCVSRGEQVEQRPPHLSVREGDSAVI | (SEQ ID |
| Control | TCTYTDPNSYYFFWYKQEPGASLQLLMKVFSSTEINEGQGFT | NO: 100) |
| TCR (TRAV3-3*01/ | VLLNKKDKRLSLNLTAAHPGDSAAYFCAVSGGTNSAGNKLT | |
| TR BV4*01) | FGIGTRVLVRPDIQNPEPAVYQLKDPRSQDSTLCLFTDFDSQIN | |
| | VPKTMESGTFITDKTVLDMKAMDSKSNGAIAWSNQTSFTCQD | |
| | IFKETNATYPSSDVPCDATLTEKSFETDMNLNFQNLSVMGLRI | |
| | LLLKVAGFNLLMTLRLWSSRAKRSGSGATNFSLLKQAGDVEE | |
| | NPGPMGCRLLSCVAFCLLGIGPLETAVFQTPNYHVTQVGNEV | |
| | SFNCKQTLGHDTMYWYKQDSKKLLKIMFSYNNKQLIVNETV | |
| | PRRFSPQSSDKAHLNLRIKSVEPEDSAVYLCASSRDWGPAEQF | |
| | FGPGTRLTVLEDLRNVTPPKVSLFEPSKAEIANKQKATLVCLA | |
| | RGFFPDHVELSWWVNGKEVHSGVSTDPQAYKESNYSYCLSS | |
| | RLRVSATFWHNPRNHFRCQVQFHGLSEEDKWPEGSPKPVTQ | |
| | NISAEAWGRADCGITSASYHQGVLSATILYEILLGKATLYAVL | |
| | VSGLVLMAMVKKKNS (SEQ ID NO: 90) | |
| HLA- | MDMRVPAQLLGLLLLWLRGARCQVQLQESGPGLVKPSQTLS | (SEQ ID |
| A*11 | LTCTVSGGSISSGGYYWSWIRQPPGKGLEWIGYIYYSGSTYYN | NO: 101) |
| CAR-1 | PSLKSRVTISVDTSKNQFSLKLSSVTAADTAVYYCARHYYYY | |
| | YMDVWGKGTTVTVSSGGGGSGGGGSGGGGSGGDIQMTQSPS | |
| | SLSASVGDRVTITCRASQSISSYLNWYQQKPGKAPKLLIYAAS | |
| | SLQSGVPSRFSGSGSGTDFTLTISSLQPEDFATYYCQQSYSTPL | |
| | TFGGGTKVEIKTTTPAPRPPTPAPTIASQPLSLRPEACRPAAGG | |
| | AVHTRGLDFACDFWVLVVVGGVLACYSLLVTVAFIIFWVRSK | |
| | RSRLLHSDYMNMTPRRPGPTRKHYQPYAPPRDFAAYRSKRG | |
| | RKKLLYIFKQPFMRPVQTTQEEDGCSCRFPEEEEGGCELRVKF | |
| | SRSADAPAYKQGQNQLYNELNLGRREEYDVLDKRRGRDPEM | |
| | GGKPRRKNPQEGLYNELQKDKMAEAYSEIGMKGERRRGKGH | |
| | DGLYQGLSTATKDTYDALHMQALPPR (SEQ ID NO: 91) | |
| HLA- | MDMRVPAQLLGLLLLWLRGARCQITLKESGPTLVKPTQTLTL | (SEQ ID |
| A*11 | TCTFSGFSLSTSGVGVGWIRQPPGKALEWLALIYWNDDKRYS | NO: 102) |
| CAR-2 | PSLKSRLTITKDTSKNQVVLTMTNMDPVDTATYYCAHKTTSF | |
| | YFDYWGQGTLVTVSSGGGGSGGGGSGGGGSGGDIQMTQSPS | |
| | SLSASVGDRVTITCRASQSISSYLNWYQQKPGKAPKLLIYAAS | |
| | SLQSGVPSRFSGSGSGTDFTLTISSLQPEDFATYYCQQSYSTPL | |
| | TFGGGTKVEIKTTTPAPRPPTPAPTIASQPLSLRPEACRPAAGG | |
| | AVHTRGLDFACDFWVLVVVGGVLACYSLLVTVAFIIFWVRSK | |
| | RSRLLHSDYMNMTPRRPGPTRKHYQPYAPPRDFAAYRSKRG | |
| | RKKLLYIFKQPFMRPVQTTQEEDGCSCRFPEEEEGGCELRVKF | |
| | SRSADAPAYKQGQNQLYNELNLGRREEYDVLDKRRGRDPEM | |
| | GGKPRRKNPQEGLYNELQKDKMAEAYSEIGMKGERRRGKGH | |
| | DGLYQGLSTATKDTYDALHMQALPPR (SEQ ID NO: 92) | |
| HLA- | MDMRVPAQLLGLLLLWLRGARCQVQLQESGPGLVKPSQTLS | (SEQ ID |
| A*11 | LTCTVSGGSISSGGYYWSWIRQPPGKGLEWIGYIYYSGSTYYN | NO: 103) |
| CAR-3 | PSLKSRVTISVDTSKNQFSLKLSSVTAADTAVYYCARHYYYY | |
| | MDVWGKGTTVTVSSGGGGSGGGGSGGGGSGGDIQMTQSPSS | |
| | LSASVGDRVTITCRASQSISSYLNWYQQKPGKAPKLLIYAASS | |
| | LQSGVPSRFSGSGSGTDFTLTISSLQPEDFATYYCQQSYSTPLT | |
| | FGGGTKVEIKTTTPAPRPPTPAPTIASQPLSLRPEACRPAAGGA | |
| | VHTRGLDFACDFWVLVVVGGVLACYSLLVTVAFIIFWVRSKR | |
| | SRLLHSDYMNMTPRRPGPTRKHYQPYAPPRDFAAYRSKRGR | |
| | KKLLYIFKQPFMRPVQTTQEEDGCSCRFPEEEEGGCELRVKFS | |
| | RSADAPAYKQGQNQLYNELNLGRREEYDVLDKRRGRDPEM | |
| | GGKPRRKNPQEGLYNELQKDKMAEAYSEIGMKGERRRGKGH | |
| | DGLYQGLSTATKDTYDALHMQALPPR (SEQ ID NO: 93) | |
| HLA- | MDMRVPAQLLGLLLLWLRGARCEVQLVESGGGLVQPGGSLR | (SEQ ID |
| A*11 | LSCAASGFTFSSYWMHWVRQAPGKGLVWVSRINSDGSSTSY | NO: 104) |
| CAR-4 | ADSVKGRFTISRDNAKNTLYLQMNSLRAEDTAVYYCCLGVL | |
| | LYNWFDPWGQGTLVTVSSGGGGSGGGGSGGGGSGGDIQMT | |
| | QSPSSLSASVGDRVTITCRASQSISSYLNWYQQKPGKAPKLLIY | |
| | AASSLQSGVPSRFSGSGSGTDFTLTISSLQPEDFATYYCQQSYS | |
| | TPLTFGGGTKVEIKTTTPAPRPPTPAPTIASQPLSLRPEACRPAA | |
| | GGAVHTRGLDFACDFWVLVVVGGVLACYSLLVTVAFIIFWV | |
| | RSKRSRLLHSDYMNMTPRRPGPTRKHYQPYAPPRDFAAYRSK | |
| | RGRKKLLYIFKQPFMRPVQTTQEEDGCSCRFPEEEEGGCELRV | |
| | KFSRSADAPAYKQGQNQLYNELNLGRREEYDVLDKRRGRDP | |
| | EMGGKPRRKNPQEGLYNELQKDKMAEAYSEIGMKGERRRG | |
| | KGHDGLYQGLSTATKDTYDALHMQALPPR (SEQ ID NO: 94) | |
| HLA- | MDMRVPAQLLGLLLLWLRGARCQVQLQESGPGLVKPSQTLS | (SEQ ID |
| A*11 | LTCTVSGGSISSGGYYWSWIRQPPGKGLEWIGYIYYSGSTYYN | NO: 105) |
| CAR-5 | PSLKSRVTISVDTSKNQFSLKLSSVTAADTAVYYCARHYYYY | |
| | YLDVWGKGTTVTVSSGGGGSGGGGSGGGGSGGDIQMTQSPS | |
| | SLSASVGDRVTITCRASQSISSYLNWYQQKPGKAPKLLIYAAS | |
| | SLQSGVPSRFSGSGSGTDFTLTISSLQPEDFATYYCQQSYSTPL | |
| | TFGGGTKVEIKTTTPAPRPPTPAPTIASQPLSLRPEACRPAAGG | |
| | AVHTRGLDFACDFWVLVVVGGVLACYSLLVTVAFIIFWVRSK | |
| | RSRLLHSDYMNMTPRRPGPTRKHYQPYAPPRDFAAYRSKRG | |
| | RKKLLYIFKQPFMRPVQTTQEEDGCSCRFPEEEEGGCELRVKF | |
| | SRSADAPAYKQGQNQLYNELNLGRREEYDVLDKRRGRDPEM | |
| | GGKPRRKNPQEGLYNELQKDKMAEAYSEIGMKGERRRGKGH | |
| | DGLYQGLSTATKDTYDALHMQALPPR (SEQ ID NO: 95) | |
| HLA- | MDMRVPAQLLGLLLLWLRGARCEVQLVESGGGLVQPGGSLR | (SEQ ID |
| A*11 | LSCAASGFTFSSYDMHWVRQATGKGLEWVSAIGTAGDTYYP | NO: 106) |
| CAR-6 | GSVKGRFTISRENAKNSLYLQMNSLRAGDTAVYYCARDLPGS | |
| | YWYFDLWGRGTLVTVSSGGGGSGGGGSGGGGSGGDIQMTQ | |
| | SPSSLSASVGDRVTITCRASQSISSYLNWYQQKPGKAPKLLIYA | |
| | ASSLQSGVPSRFSGSGSGTDFTLTISSLQPEDFATYYCQQSYST | |
| | PLTFGGGTKVEIKTTTPAPRPPTPAPTIASQPLSLRPEACRPAAG | |
| | GAVHTRGLDFACDFWVLVVVGGVLACYSLLVTVAFIIFWVRS | |
| | KRSRLLHSDYMNMTPRRPGPTRKHYQPYAPPRDFAAYRSKR | |
| | GRKKLLYIFKQPFMRPVQTTQEEDGCSCRFPEEEEGGCELRVK | |
| | FSRSADAPAYKQGQNQLYNELNLGRREEYDVLDKRRGRDPE | |
| | MGGKPRRKNPQEGLYNELQKDKMAEAYSEIGMKGERRRGK | |
| | GHDGLYQGLSTATKDTYDALHMQALPPR (SEQ ID NO: 96) | |
| HLA- | MDMRVPAQLLGLLLLWLRGARCQVQLVQSGAEVKKPGASV | (SEQ ID |
| A*11 | KVSCKASGYTFTSYAMHWVRQAPGQRLEWMGWINAGNGNT | NO: 107) |
| CAR-7 | KYSQKFQGRVTITRDTSASTAYMELSSLRSEDTAVYYCAREG | |
| | NGANPDAFDIWGQGTMVTVSSGGGGSGGGGSGGGGSGGDIQ | |
| | MTQSPSSLSASVGDRVTITCRASQSISSYLNWYQQKPGKAPKL | |
| | LIYAASSLQSGVPSRFSGSGSGTDFTLTISSLQPEDFATYYCQQ | |
| | SYSTPLTFGGGTKVEIKTTTPAPRPPTPAPTIASQPLSLRPEACR | |
| | PAAGGAVHTRGLDFACDFWVLVVVGGVLACYSLLVTVAFIIF | |
| | WVRSKRSRLLHSDYMNMTPRRPGPTRKHYQPYAPPRDFAAY | |
| | RSKRGRKKLLYIFKQPFMRPVQTTQEEDGCSCRFPEEEEGGCE | |
| | LRVKFSRSADAPAYKQGQNQLYNELNLGRREEYDVLDKRRG | |
| | RDPEMGGKPRRKNPQEGLYNELQKDKMAEAYSEIGMKGERR | |
| | RGKGHDGLYQGLSTATKDTYDALHMQALPPR (SEQ ID NO: | |
| | 97) | |
| HLA- | MDMRVPAQLLGLLLLWLRGARCQITLKESGPTLVKPTQTLTL | (SEQ ID |
| A*11 | TCTFSGFSLSTSGVGVGWIRQPPGKALEWLALIYWNDDKRYS | NO: 108) |
| CAR-8 | PSLKSRLTITKDTSKNQVVLTMTNMDPVDTATYYCAHRHMR | |
| | LSCFDYWGQGTLVTVSSGGGGSGGGGSGGGGSGGDIQMTQS | |
| | PSSLSASVGDRVTITCRASQSISSYLNWYQQKPGKAPKLLIYA | |
| | ASSLQSGVPSRFSGSGSGTDFTLTISSLQPEDFATYYCQQSYST | |
| | PLTFGGGTKVEIKTTTPAPRPPTPAPTIASQPLSLRPEACRPAAG | |
| | GAVHTRGLDFACDFWVLVVVGGVLACYSLLVTVAFIIFWVRS | |
| | KRSRLLHSDYMNMTPRRPGPTRKHYQPYAPPRDFAAYRSKR | |
| | GRKKLLYIFKQPFMRPVQTTQEEDGCSCRFPEEEEGGCELRVK | |
| | FSRSADAPAYKQGQNQLYNELNLGRREEYDVLDKRRGRDPE | |
| | MGGKPRRKNPQEGLYNELQKDKMAEAYSEIGMKGERRRGK | |
| | GHDGLYQGLSTATKDTYDALHMQALPPR (SEQ ID NO: 98) | |
| HLA- | MDMRVPAQLLGLLLLWLRGARCQVQLQESGPGLVKPSQTLS | (SEQ ID |
| A*11 | LTCTVSGGSISSGGYYWSWIRQPPGKGLEWIGYIYYSGSTYYN | NO: 109) |
| CAR-9 | PSLKSRVTISVDTSKNQFSLKLSSVTAADTAVYYCARHYYYY | |
| | SMDVWGKGTTVTVSSGGGGSGGGGSGGGGSGGDIQMTQSPS | |
| | SLSASVGDRVTITCRASQSISSYLNWYQQKPGKAPKLLIYAAS | |
| | SLQSGVPSRFSGSGSGTDFTLTISSLQPEDFATYYCQQSYSTPL | |
| | TFGGGTKVEIKTTTPAPRPPTPAPTIASQPLSLRPEACRPAAGG | |
| | AVHTRGLDFACDFWVLVVVGGVLACYSLLVTVAFIIFWVRSK | |
| | RSRLLHSDYMNMTPRRPGPTRKHYQPYAPPRDFAAYRSKRG | |
| | RKKLLYIFKQPFMRPVQTTQEEDGCSCRFPEEEEGGCELRVKF | |
| | SRSADAPAYKQGQNQLYNELNLGRREEYDVLDKRRGRDPEM | |
| | GGKPRRKNPQEGLYNELQKDKMAEAYSEIGMKGERRRGKGH | |
| | DGLYQGLSTATKDTYDALHMQALPPR (SEQ ID NO: 99) | |

### Example 3: HLA-A*11 Titration in HeLa Target Cells and HLA-A*11 CAR Activation

### Cell Lines and Peptides

HeLa cells were purchased from ATCC®. Jurkat NFAT-Firefly-Luciferase (JNL) cells were purchased from BPS Bioscience. All cell lines were cultured in the media as recommended by the vendors.

mRNA Production

HLA-A*11:01 mRNA was synthesized using a PCR product containing 5′ T7 promoter as template. In vitro transcription was performed using the HiScribe T7 ARCA mRNA Kit (New England Biolabs (NEB) E2060S) according to manufacturer's protocol supplemented with 1.25 mM modified pseudo-UTP (TriLink). After in vitro transcription, 2 uL of DNase I (NEB M0303S) was added to the reaction and incubated for 15 min at 37° C. The mRNA was polyadenylated using polyA enzyme (NEB M0276S) for 30 minutes at 37° C., then cleaned up using the NEB Monarch RNA Clean Up Kit (NEB T2040L) according to manufacturer's protocol. The purified mRNA was then treated with antarctic phosphatase (NEB M0289S). Final purification was performed using the NEB Monarch RNA Clean Up Kit. The mRNA was eluted in 1 mM sodium acetate, pH 6.4, and stored at −80° C.

HeLa Cell mRNA Titration

On day 1, JNL cells were transfected with appropriate effector molecules as described above. To titrate HLA-A*11:01 molecules on target cells, HLA-A*11:01 mRNA was serially diluted two-fold, from 2000 ng to 0.24 ng per well, in SE buffer (Lonza V4XC-1024). HeLa cells were transfected with the serially-diluted HLA-A*11:01 mRNA using standard HeLa protocol with the 4D-Nucleofector™ X Unit (Lonza AAF-1002X). Transfected HeLa cells were incubated overnight at 37° C. in 384 well plates, plated at 10,000 cells per well (Corning 3570). On day 2, 10,000 transfected JNL cells were added to each well containing the mRNA transfected-HeLa cells to a final volume of 30 μL. After a 6-hour incubation at 37° C., NFAT response was measured as described above.

### Target Cells Flow Analysis

To quantify the number of HLA-A*11:01 molecules on the surface of the transfected HeLa cells, ˜80,000 mRNA-transfected HeLa cells were detached by adding 100 uL of FACS buffer (1×PBS supplemented with 1% BSA) and pipetting up and down ˜5×. Cells were washed 2× with 100 uL FACS buffer, then resuspended in 100 uL solution containing HLA-A*11:01 monoclonal antibody (OneLamda 0284HA) diluted 1:50 in FACS buffer for 30 minutes on ice. The cells, along with quantification beads provided by the QIFIKIT (Agilent K0078), were washed 2× with 100 uL FACS buffer. The cells and beads were stained with 1:50 diluted secondary antibody provided by the QIFIKIT for 45 minutes on ice. After washing 2× with 100 uL FACS buffer, the cells and beads were analyzed by flow cytometry and quantified according to manufacturer's protocol.

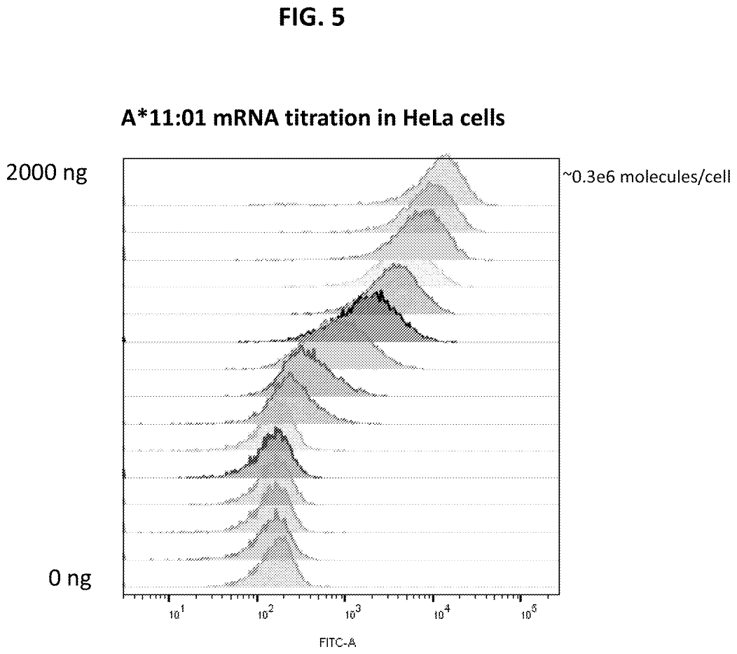

Quantification of mRNA titration and HLA-A*11:01 expression by HeLa cells is shown in FIG. 5. As seen in FIG. 5, HLA-A*11:01 expression by HeLa cells correlates with the amount of HLA-A*11:01 mRNA used to transfect the cells.

Jurkat NFAT-Luciferase effector cells were transfected, and their activation was assayed as described above for Example 2, except that HeLa cells were used instead of T2 target cells.

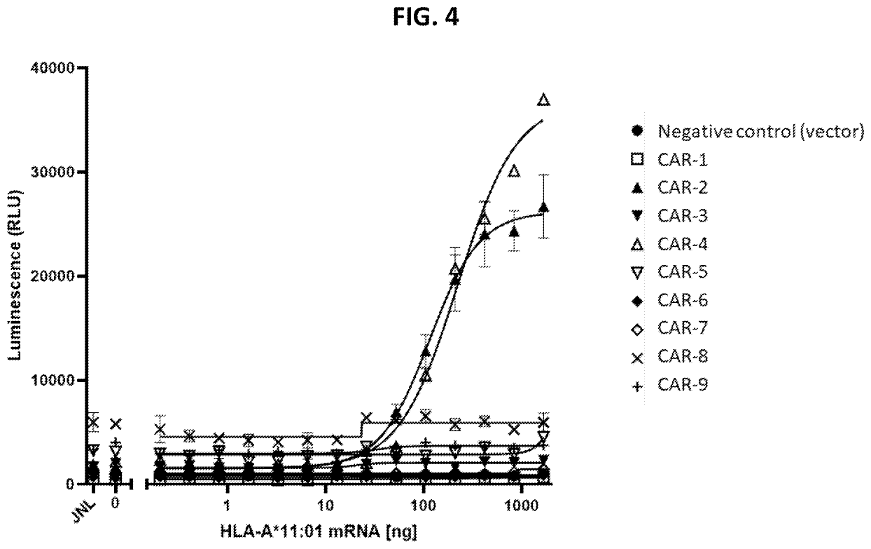

Results of Jurkat NFAT-Luciferase activation assays using HeLa target cells are shown in FIG. 4. As see in FIG. 4, both CAR-2 and CAR-4 showed robust activation of Jurkat effector cells when the cells were co-cultured with HeLa target cells expressing HLA-A*11:01.

### Example 4: HLA-A*11 scFv in an Inhibitory Receptor Context

An HLA-A*11 inhibitory receptor was created by fusing scFv #4 (Table) to a hinge, TM and ICD derived from LIR-1. Gene segments were combined using Golden Gate cloning and inserted downstream of a human EF1alpha promoter contained in a lentivirus expression plasmid.

Jurkat NFAT-Luciferase effector cells were transformed as described above with a CAR activator, or a CAR activator and the scFv #4 HLA-A*11 inhibitory (blocker) receptor. Sequences of the HLA-A*11 inhibitory receptor are shown in Table 7, below. and co-cultured with HeLa cells expressing both the activator ligand and HLA-A*11:01. HeLa cells were prepared as described in Example 3, NFAT-Luciferase assays were carried out as described in Example 2, using HeLa instead of T2 target cells.

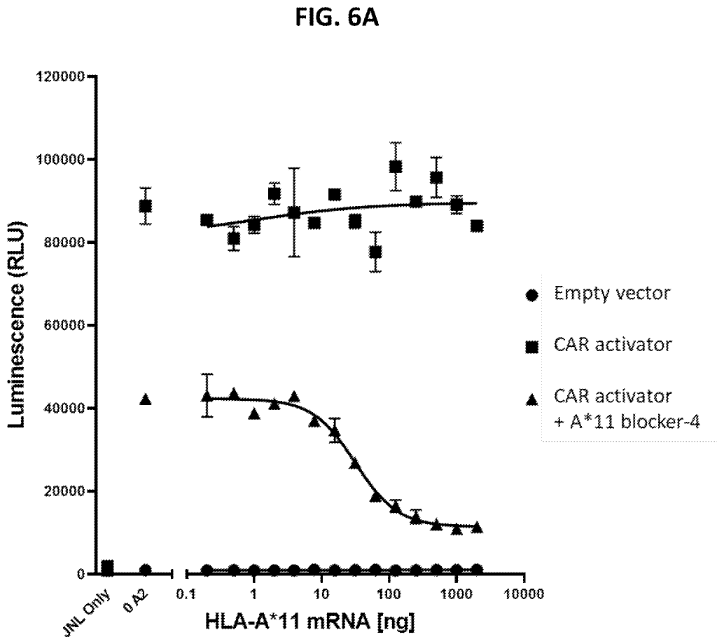

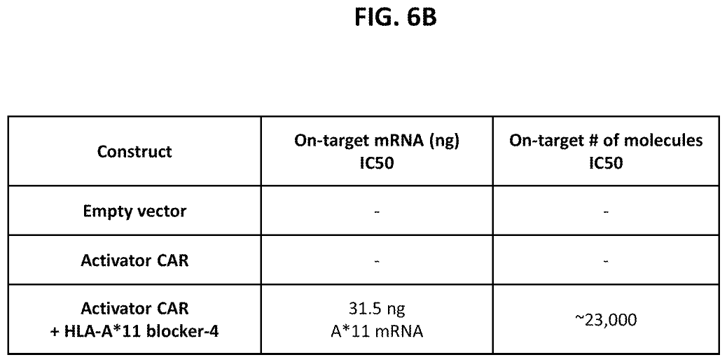

Results are shown in FIGS. 6A-6B. As shown in FIG. 6A, scFv #4, when fused to LIR-1's hinge, TM and ICD domains, is capable of inhibiting activation of Jurkat effector cells co-cultured with target cells expressing both activator ligand and HLA-A*11.

TABLE 7
HLA-A*11 inhibitory receptor sequences.

| | | DNA |
|---|---|---|
| Name | Protein Sequence | Sequence |
| HLA- | MDMRVPAQLLGLLLLWLRGARCEVQLVESGGGLVQPGG | (SEQ ID |
| A*11 | SLRLSCAASGFTFSSYWMHWVRQAPGKGLVWVSRINSDGS | NO: 112) |
| Blocker | STSYADSVKGRFTISRDNAKNTLYLOMNSLRAEDTAVYYCC | |
| 4 | LGVLLYNWFDPWGQGTLVTVSSGGGGSGGGGSGGGGSG | |
| | GDIQMTQSPSSLSASVGDRVTITCRASQSISSYLNWYQQKPG | |
| | KAPKLLIYAASSLQSGVPSRFSGSGSGTDFTLTISSLQPEDFA | |
| | TYYCQQSYSTPLTFGGGTKVEIKYGSQSSKPYLLTHPSDPLE | |
| | LVVSGPSGGPSSPTTGPTSTSGPEDQPLTPTGSDPQSGLGRHL | |
| | GVVIGILVAVILLLLLLLLLFLILRHRRQGKHWTSTQRKADF | |
| | QHPAGAVGPEPTDRGLQWRSSPAADAQEENLYAAVKHTQP | |
| | EDGVEMDTRSPHDEDPQAVTYAEVKHSRPRREMASPPSPLS | |
| | GEFLDTKDRQAEEDRQMDTEAAASEAPQDVTYAQLHSLTL | |
| | RREATEPPPSQEGPSPAVPSIYATLAIH (SEQ ID NO: 111) | |

What is claimed is:

1. A polypeptide, comprising an antigen binding domain that specifically binds to a major histocompatibility class I (MHC I) complex comprising a human leukocyte antigen a chain encoded by an HLA-A*11 allele (HLA-A*11).

2. The polypeptide of claim 1, wherein the antigen binding domain is a human antibody or an antigen-binding fragment thereof.

3. The polypeptide of claim 1, wherein the antigen binding domain comprises a single chain variable fragment (scFv), a single chain Fab (scFab), a single domain antibody (sdAb), a fragment antigen binding (Fab), a F(ab′)₂, or a Fab′.

4. The polypeptide of claim 1, wherein the antigen binding domain is a scFv.

5. The polypeptide of claim 4, wherein the scFv comprises a variable heavy chain (VH)-linker-variable light chain (VL) or a VL-linker-VH orientation.

6. The polypeptide of any one of claims 1-5, wherein the antigen binding domain comprises:
a. a heavy chain (HC) complementarity determining region 1 (CDR1) sequence selected from the group consisting of SGGYYWS (SEQ ID NO: 1), TSGVGVG (SEQ ID NO: 2), SYAMH (SEQ ID NO: 3), SYDMH (SEQ ID NO: 4), and SYWMH (SEQ ID NO: 5);
b. a HC CDR2 sequence selected from the group consisting of YIYYSGSTYYNPSLKS (SEQ ID NO: 6), LIYWNDDKRYSPSLKS (SEQ ID NO: 7), WINAGNGNTKYSQKFQG (SEQ ID NO: 8), AIGTAGDTYYPGSVKG (SEQ ID NO: 9), and RINSDGSSTSYADSVKG (SEQ ID NO: 10); and
c. a HC CDR3 sequence selected from the group consisting of HYYYYSMDV (SEQ ID NO: 11), HYYYYYLDV (SEQ ID NO: 12), HYYYYMDV (SEQ ID NO: 13), HYYYYYMDV (SEQ ID NO: 14), KTTSFYFDY (SEQ ID NO: 15), RHMRLSCFDY (SEQ ID NO: 16), EGNGANPDAFDI (SEQ ID NO: 17), DLPGSYWYFDL (SEQ ID NO: 18), and GVLLYNWFDP (SEQ ID NO: 19).

7. The polypeptide of any one of claims 1-6, wherein the antigen binding domain comprises a light chain (LC) complementarity determining region 1 (CDR1) comprising a sequence of RASQSISSYLN (SEQ ID NO: 20), a LC CDR2 comprising a sequence of AASSLQS (SEQ ID NO: 21) and a LC CDR3 comprising a sequence of QQSYSTPLT (SEQ ID NO: 22).

8. The polypeptide of any one of claims 1-5, wherein the antigen binding domain comprises a HC CDR1 comprising TSGVGVG (SEQ ID NO: 2), a HC CDR2 comprising LIYWNDDKRYSPSLKS (SEQ ID NO: 7), a HC CDR3 comprising KTTSFYFDY (SEQ ID NO: 15), a LC CDR1 comprising RASQSISSYLN (SEQ ID NO: 20), a LC CDR2 comprising AASSLQS (SEQ ID NO: 21), and a LC CDR3 comprising QQSYSTPLT (SEQ ID NO: 22).

9. The polypeptide of any one of claims 1-5, wherein the antigen binding domain comprises a HC CDR1 comprising SYWMH (SEQ ID NO: 5), a HC CDR2 comprising RINSDGSSTSYADSVKG (SEQ ID NO: 10), a HC CDR comprising GVLLYNWFDP (SEQ ID NO: 19), a LC CDR1 comprising RASQSISSYLN (SEQ ID NO: 20), a LC CDR2 comprising AASSLQS (SEQ ID NO: 21), and a LC CDR3 comprising QQSYSTPLT (SEQ ID NO: 22).

10. The polypeptide of any one of claims 1-5, wherein the antigen binding domain comprises a variable heavy chain comprising a sequence of, or a functional variant thereof having at least 90%, at least 95%, at least 97%, at least 98% or at least 99% sequence identity to:
a.
(SEQ ID NO: 42)
QVQLQESGPGLVKPSQTLSLTCTVSGGSISSGGYYWSWIRQPPGKGLE
WIGYIYYSGSTYYNPSLKSRVTISVDTSKNQFSLKLSSVTAADTAVYY
CARHYYYYSMDVWGKGTTVTVSS;
b.
(SEQ ID NO: 43)
QITLKESGPTLVKPTQTLTLTCTFSGFSLSTSGVGVGWIRQPPGKALEW
LALIYWNDDKRYSPSLKSRLTITKDTSKNQVVLTMTNMDPVDTATYY
CAHRHMRLSCFDYWGQGTLVTVSS;
c.
(SEQ ID NO: 44)
QVQLVQSGAEVKKPGASVKVSCKASGYTFTSYAMHWVRQAPGQRLE
WMGWINAGNGNTKYSQKFQGRVTITRDTSASTAYMELSSLRSEDTAV
YYCAREGNGANPDAFDIWGQGTMVTVSS;
d.
(SEQ ID NO: 45)
EVQLVESGGGLVQPGGSLRLSCAASGFTFSSYDMHWVRQATGKGLE
WVSAIGTAGDTYYPGSVKGRFTISRENAKNSLYLQMNSLRAGDTAVY
YCARDLPGSYWYFDLWGRGTLVTVSS;
e.
(SEQ ID NO: 46)
QVQLQESGPGLVKPSQTLSLTCTVSGGSISSGGYYWSWIRQPPGKGLE
WIGYIYYSGSTYYNPSLKSRVTISVDTSKNQFSLKLSSVTAADTAVYY
CARHYYYYYLDVWGKGTTVTVSS;
f.
(SEQ ID NO: 47)
EVQLVESGGGLVQPGGSLRLSCAASGFTFSSYWMHWVRQAPGKGLV
WVSRINSDGSSTSYADSVKGRFTISRDNAKNTLYLQMNSLRAEDTAV
YYCCLGVLLYNWFDPWGQGTLVTVSS;
g.
(SEQ ID NO: 48)
QVQLQESGPGLVKPSQTLSLTCTVSGGSISSGGYYWSWIRQPPGKGLE
WIGYIYYSGSTYYNPSLKSRVTISVDTSKNQFSLKLSSVTAADTAVYY
CARHYYYYMDVWGKGTTVTVSS;
h.
(SEQ ID NO: 49)
QITLKESGPTLVKPTQTLTLTCTFSGFSLSTSGVGVGWIRQPPGKALEW
LALIYWNDDKRYSPSLKSRLTITKDTSKNQVVLTMTNMDPVDTATYY
CAHKTTSFYFDYWGQGTLVTVSS;
or
i.
(SEQ ID NO: 50)
QVQLQESGPGLVKPSQTLSLTCTVSGGSISSGGYYWSWIRQPPGKGLE
WIGYIYYSGSTYYNPSLKSRVTISVDTSKNQFSLKLSSVTAADTAVYY
CARHYYYYYMDVWGKGTTVTVSS.

11. The polypeptide of claim 10, wherein the antigen binding domain comprises a variable light chain comprising a sequence of, or a functional variant thereof having at least 95%, at least 97%, at least 98% or at least 99% sequence identity to:
(SEQ ID NO: 51)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPLTF
GGGTKVEIK.

12. The polypeptide of anyone of claims 1-5, wherein the antigen binding domain comprises a variable heavy chain of SEQ ID NO: 47 and a variable light chain of SEQ ID NO: 51, or a functional variant thereof having at least 90%, at least 95%, at least 97%, at least 98% or at least 99% sequence identity thereto.

13. The polypeptide of anyone of claims 1-5, wherein the antigen binding domain comprises a variable heavy chain of SEQ ID NO: 49 and a variable light chain of SEQ ID NO: 51, or a functional variant thereof having at least 95%, at least 97%, at least 98% or at least 99% sequence identity thereto.

14. The polypeptide of anyone of claims 1-5, wherein the antigen binding domain comprises a sequence of, or a functional variant thereof having at least 95%, at least 97%, at least 98% or at least 99% sequence identity to:
a.
(SEQ ID NO: 23)
QVQLQESGPGLVKPSQTLSLTCTVSGGSISSGGYYWSWIRQPPGKGLE
WIGYIYYSGSTYYNPSLKSRVTISVDTSKNQFSLKLSSVTAADTAVYY
CARHYYYYSMDVWGKGTTVTVSSGGGGSGGGGSGGGGSGGDIQMT
QSPSSLSASVGDRVTITCRASQSISSYLNWYQQKPGKAPKLLIYAASSL
QSGVPSRFSGSGSGTDFTLTISSLQPEDFATYYCQQSYSTPLTFGGGTK
VEIK;
b.
(SEQ ID NO: 24)
QITLKESGPTLVKPTQTLTLTCTFSGFSLSTSGVGVGWIRQPPGKALEW
LALIYWNDDKRYSPSLKSRLTITKDTSKNQVVLTMTNMDPVDTATYY
CAHRHMRLSCFDYWGQGTLVTVSSGGGGSGGGGSGGGGSGGDIQMT
QSPSSLSASVGDRVTITCRASQSISSYLNWYQQKPGKAPKLLIYAASSL
QSGVPSRFSGSGSGTDFTLTISSLQPEDFATYYCQQSYSTPLTFGGGTK
VEIK;
c.
(SEQ ID NO: 25)
QVQLVQSGAEVKKPGASVKVSCKASGYTFTSYAMHWVRQAPGQRLE
WMGWINAGNGNTKYSQKFQGRVTITRDTSASTAYMELSSLRSEDTAV
YYCAREGNGANPDAFDIWGQGTMVTVSSGGGGSGGGGGGGGSGG
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPLTFG
GGTKVEIK;
d.
(SEQ ID NO: 26)
EVQLVESGGGLVQPGGSLRLSCAASGFTFSSYDMHWVRQATGKGLE
WVSAIGTAGDTYYPGSVKGRFTISRENAKNSLYLQMNSLRAGDTAVY
YCARDLPGSYWYFDLWGRGTLVTVSSGGGGSGGGGSGGGGSGGDIQ
MTQSPSSLSASVGDRVTITCRASQSISSYLNWYQQKPGKAPKLLIYAAS
SLQSGVPSRFSGSGSGTDFTLTISSLQPEDFATYYCQQSYSTPLTFGGGT
KVEIK;
e.
(SEQ ID NO: 27)
QVQLQESGPGLVKPSQTLSLTCTVSGGSISSGGYYWSWIRQPPGKGLE
WIGYIYYSGSTYYNPSLKSRVTISVDTSKNQFSLKLSSVTAADTAVYY
CARHYYYYYLDVWGKGTTVTVSSGGGGSGGGGSGGGGSGGDIQMT
QSPSSLSASVGDRVTITCRASQSISSYLNWYQQKPGKAPKLLIYAASSL
QSGVPSRFSGSGSGTDFTLTISSLQPEDFATYYCQQSYSTPLTFGGGTK
VEIK;
f.
(SEQ ID NO: 28)
EVQLVESGGGLVQPGGSLRLSCAASGFTFSSYWMHWVRQAPGKGLV
WVSRINSDGSSTSYADSVKGRFTISRDNAKNTLYLQMNSLRAEDTAV
YYCCLGVLLYNWFDPWGQGTLVTVSSGGGGSGGGGSGGGGSGGDIQ
MTQSPSSLSASVGDRVTITCRASQSISSYLNWYQQKPGKAPKLLIYAAS
SLQSGVPSRFSGSGSGTDFTLTISSLQPEDFATYYCQQSYSTPLTFGGGT
KVEIK;
g.
(SEQ ID NO: 29)
QVQLQESGPGLVKPSQTLSLTCTVSGGSISSGGYYWSWIRQPPGKGLE
WIGYIYYSGSTYYNPSLKSRVTISVDTSKNQFSLKLSSVTAADTAVYY
CARHYYYYMDVWGKGTTVTVSSGGGGSGGGGSGGGGSGGDIQMTQ
SPSSLSASVGDRVTITCRASQSISSYLNWYQQKPGKAPKLLIYAASSLQ
SGVPSRFSGSGSGTDFTLTISSLQPEDFATYYCQQSYSTPLTFGGGTKV
EIK;
h.
(SEQ ID NO: 30)
QITLKESGPTLVKPTQTLTLTCTFSGFSLSTSGVGVGWIRQPPGKALEW
LALIYWNDDKRYSPSLKSRLTITKDTSKNQVVLTMTNMDPVDTATYY
CAHKTTSFYFDYWGQGTLVTVSSGGGGSGGGGSGGGGSGGDIQMTQ
SPSSLSASVGDRVTITCRASQSISSYLNWYQQKPGKAPKLLIYAASSLQ
SGVPSRFSGSGSGTDFTLTISSLQPEDFATYYCQQSYSTPLTFGGGTKV
EIK;
or
i.
(SEQ ID NO: 31)
QVQLQESGPGLVKPSQTLSLTCTVSGGSISSGGYYWSWIRQPPGKGLE
WIGYIYYSGSTYYNPSLKSRVTISVDTSKNQFSLKLSSVTAADTAVYY
CARHYYYYYMDVWGKGTTVTVSSGGGGSGGGGSGGGGSGGDIQMT
QSPSSLSASVGDRVTITCRASQSISSYLNWYQQKPGKAPKLLIYAASSL
QSGVPSRFSGSGSGTDFTLTISSLQPEDFATYYCQQSYSTPLTFGGGTK
VEIK.

15. The polypeptide of anyone of claims 1-5, wherein the antigen binding domain comprises a sequence of SEQ ID NO: 28 or SEQ ID NO: 30, or a functional variant thereof having at least at least 90%, at least 95%, at least 97%, at least 98% or at least 99% sequence identity thereto.

16. The polypeptide of any one of claims 1-13, wherein the polypeptide comprises a monoclonal antibody.

17. The polypeptide of any one of claims 1-13, wherein the polypeptide comprises a multispecific antibody.

18. A receptor comprising the polypeptide of anyone of claims 1-15.

19. The receptor of claim 18, wherein the receptor provides an activating signal to a cell.

20. The receptor of claim 19, wherein the cell is an immune cell.

21. The receptor of claim 19 or 20, wherein the receptor is a chimeric antigen receptor (CAR) or a T Cell Receptor (TCR).

22. The receptor of claim 21, wherein the CAR comprises an extracellular antigen-binding domain, a transmembrane domain, and one or more intracellular domains, and wherein the extracellular antigen-binding domain comprises the polypeptide.

23. The receptor of claim 22, wherein the transmembrane domain comprises a transmembrane domain isolated or derived from CD8a molecule (CD8α), CD4 molecule (CD4), CD28 molecule (CD28), TNF receptor superfamily member 9 (CD137, or 4-1BB), CD80 molecule (CD80), CD86 molecule (CD86), cytotoxic T-lymphocyte associated protein 4 (CD152), programmed cell death 1 (PD-1), CD247 molecule (CD3ζ), or Fc fragment of IgE receptor Ig (FcRγ).

24. The receptor of claim 22 or 23, wherein the one or more intracellular domains comprise an intracellular signaling domain isolated or derived from an immune effector cell protein.

25. The receptor of claim 24, wherein the intracellular signaling domain comprises an intracellular signaling domain isolated or derived from CD3ζ.

26. The receptor of any one of claims 22-25, wherein the CAR comprises a co-stimulatory domain.

27. The receptor of claim 26, wherein the co-stimulatory domain comprises a co-stimulatory domain isolated or derived from CD27 molecule (CD27), CD28, CD137, TNF receptor superfamily member 4 (OX40), TNF receptor superfamily member 8 (CD30), CD40 molecule (CD40), CD40 ligand (CD40L), CD3ζ, integrin subunit beta 2 (LFA-1), inducible T cell costimulator (ICOS), CD2 molecule (CD2), CD7 molecule (CD7), TNF superfamily member 14 (LIGHT), killer cell lectin like receptor C2 (NKG2C), CD276 molecule (B7-H3), or hematopoietic cell signal transducer (DAP10).

28. The receptor of any one of claims 22-27, wherein the one or more intracellular domains comprise intracellular domains isolated or derived from CD28, 4-1BB and CD3ζ.

29. The receptor of any one of claims 21-28, wherein the CAR comprises a hinge domain between the extracellular domain and the transmembrane domain.

30. The receptor of claim 29, wherein the hinge domain is isolated or derived from CD4, CD8α, IgG1, IgG2, or IgG4.

31. The receptor of any one of claims 21-30, wherein the CAR comprises a signal peptide.

32. The receptor of claim 31, wherein the signal peptide comprises a sequence of MDMRVPAQLLGLLLLWLRGARC (SEQ ID NO: 63).

33. The receptor of any one of claims 29-32, wherein the hinge, transmembrane, and intracellular domains of the CAR comprise a sequence at least 90%, at least 95%, at least 99%, or 100% identical to TTTPAPRPPTPAPTIASQPLSLRPEACRPAAGGAVHTRGLDFACDFWVLVVVGGVLACY SLLVTVAFIIFWVRSKRSRLLHSDYMNMTPRRPGPTRKHYQPYAPPRDFAAYRSKRGRK KLLYIFKQPFMRPVQTTQEEDGCSCRFPEEEEGGCELRVKFSRSADAPAYKQGQNQLYN ELNLGRREEYDVLDKRRGRDPEMGGKPRRKNPQEGLYNELQKDKMAEAYSEIGMKGE RRRGKGHDGLYQGLSTATKDTYDALHMQALPPR (SEQ ID NO: 64).

34. The receptor of claim 21, wherein the TCR comprises an extracellular antigen-binding domain comprising the polypeptide.

35. The receptor of claim 34, wherein the antigen binding domain is fused to one or more of a TCRα, TCRβ, CD3ζ, CD3δ, CD3ε or CD3γ subunits of the TCR.

36. The receptor of claim 18, wherein the receptor provides an inhibitory signal to a cell.

37. The receptor of claim 36, wherein the cell is an immune cell.

38. The receptor of claim 36 or 37, wherein the receptor is an inhibitory CAR or a TCR.

39. The receptor of any one of claims 36-38, comprising an extracellular domain comprising the antibody or antigen-binding fragment thereof and an inhibitory intracellular domain.

40. The receptor of claim 39, wherein the inhibitory intracellular domain is isolated or derived from leukocyte immunoglobulin like receptor B1 (LILRB1).

41. The receptor of any one of claims 36-41, comprising a transmembrane domain.

42. The receptor of claim 41, wherein the transmembrane domain is isolated or derived from TCRα, TCRb, CD8alpha, CD28 or LILRB1.

43. The receptor of claim 42, further comprising an extracellular hinge domain.

44. The receptor of claim 43, wherein the extracellular hinge domain comprises a hinge domain isolated or derived from CD8alpha, CD28 or LILRB1.

45. The receptor of any one of claims 40-44, wherein the hinge, transmembrane, and intracellular domains comprise a sequence at least 90%, at least 95%, at least 99%, or 100% identical to YGSQSSKPYLLTHPSDPLELVVSGPSGGPSSPTTGPTSTSGPEDQPLTPTGSDPQS GLGRHLGVVIGILVAVILLLLLLLLLFLILRHRRQGKHWTSTQRKADFQHPAGAV GPEPTDRGLQWRSSPAADAQEENLYAAVKHTQPEDGVEMDTRSPHDEDPQAVT YAEVKHSRPRREMASPPSPLSGEFLDTKDRQAEEDRQMDTEAAASEAPQDVTY AQLHSLTLRREATEPPPSQEGPSPAVPSIYATLAIH (SEQ ID NO: 65).

46. A nucleic acid encoding the polypeptide of any one of claims 1-17.

47. A vector comprising the nucleic acid of claim 46.

48. A nucleic acid encoding the receptor of any one of claims 18-45.

49. A vector comprising the nucleic acid of claim 48.

50. A cell comprising the nucleic acid of claim 46 or the vector of claim 47.

51. A cell comprising the nucleic acid of claim 48 or the vector of claim 49.

52. A recombinant immune cell expressing the receptor of any one of claims 18-45.

53. The recombinant immune cell of claim 52, wherein the immune cell is a T cell, B cell, macrophage or an NK cell.

54. A pharmaceutical composition comprising the polypeptide of any one of claims 1-17 or the receptor of any one of claims 18-45, and a pharmaceutically acceptable carrier, diluent, or excipient.

55. A pharmaceutical composition comprising a plurality of the recombinant immune cell of claim 52 or 53, and a pharmaceutically acceptable carrier, diluent, or excipient.

56. A method for treating a cancer a subject in need thereof, the method comprising administering a therapeutically effective amount of the recombinant cell of claim 52 or 53, or the pharmaceutical composition of claim 54 to the subject, wherein cells of the cancer have lost expression of HLA-A*11 due to loss of heterozygosity.

57. The method of claim 56, further comprising:
a. determining if the subject is heterozygous for an HLA-A*11 allele;
b. isolating a plurality of cancer cells from the subject;
c. detecting the presence or absence of HLA-A*11 on the cancer cells using the polypeptide of any one of claims 1-17; and
d. administering the recombinant cell or pharmaceutical composition when the plurality of cancer cells do not express HLA-A*11.

58. The method of claim 56 or 57, wherein the cancer comprises a liquid tumor or a solid tumor.

59. A method for determining whether cancer cells express HLA-A*11, comprising:
a. providing a plurality of cancer cells; and
b. detecting the presence of absence of HLA-A*11 on the cancer cells using the polypeptide of any one of claims 1-17.

60. The method of claim 59, wherein the detecting at step (b) comprises immunohistochemistry.

61. A method of making a recombinant immune cell, comprising:
a. providing a plurality of immune cells; and
b. transforming the plurality of immune cells with the nucleic acid of claim 48 or the vector of claim 49.

62. A method of making a polypeptide, comprising:
a. contacting the nucleic acid of claim 46 or the vector of claim 47 with a cell;
b. culturing the cell under conditions whereby the polypeptide is expressed by the cell; and
c. purifying the polypeptide.

63. A kit, comprising the polypeptide of any one of claims 1-17, the receptor of any one of claims 18-45, the nucleic acid of claim 46 or 48, the vector of claim 47 or 49, the cell of claim 50, the recombinant immune cell of claim 51 or 52, or the pharmaceutical composition of claim 53 or 54.
